(19) United States
(12) Patent Application Publication
(10) Pub. No.: US 2005/0254663 A1
(43) Pub. Date: Nov. 17, 2005

(54) Electronic sound screening system and method of accoustically impoving the environment

(76) Inventors: Andreas Raptopoulos, (US); Volkmar Klien, Wien (AT); Nick Rothwell, London (GB); Ian Morris, London (GB); Alexander Wilkie, London (GB)

Correspondence Address: BRINKS HOFER GILSON & LIONE, P.O. BOX 10395, CHICAGO, IL, 60610, US

(21) Appl. No.: 10/996,330

(22) Filed: Nov. 23, 2004

Related U.S. Application Data
(63) Continuation-in-part of application No. 10/145,113, filed on May 15, 2002.
Application No. 10/145,113 is a continuation of application No. PCT/GB01/04234, filed on Sep. 21, 2001.

(30) Foreign Application Priority Data
Nov. 16, 1999 (GB) GB 9927131.4
Sep. 21, 2000 (GB) GB 0023207.4

(51) Int. Cl. A61F 11/06; G10K 11/16; H03B 29/00

(52) U.S. Cl. 381/71.1

(57) ABSTRACT

A flexible apparatus for, and method of, acoustically improving an environment permits manual adjustment by one or more local or remote users using a simple graphical interface and automatic adjustment of the system parameters once the manual adjustment is performed. The inputs are weighted by distance from the physical apparatus. The apparatus includes a receiver, a converter, an analyser, a processor and a sound generator. The acoustic energy impinges on the receiver and is converted to an electrical signal by the converter. The analyser receives the electrical signal from the receiver, analyzes the electrical signal, and generates data analysis signals in response to the analyzed electrical signal. The processor produces sound signals based on the data analysis signals from the analyser in each critical band. The sound generator provides sound based on the sound signals. This permits the users to define the sound heard in a set space.

### PRIORITY

This application is a continuation-in-part of U.S. application Ser. No. 10/145,113, filed Feb. 6, 2003 and entitled, “Apparatus for acoustically improving an environment,” which is a continuation of International Application PCT/GB01/04234, with an international filing date of Sep. 21, 2001, published in English under PCT Article 21(2) and U.S. application Ser. No. 10/145,097, filed Jan. 2, 2003 and entitled, “Apparatus for acoustically improving an environment and related method,” which is a continuation-in-part of International Application PCT/GB00/02360, with an international filing date of Jun. 16, 2000, published in English under PCT Article 21(2) and now abandoned. Each of the preceding applications are incorporated herein by reference in their entirety.

### BACKGROUND

The present invention relates to an apparatus for acoustically improving an environment, and particularly to an electronic sound screening system.

In order to understand the present invention, it is necessary to appreciate some relevant characteristics of the human auditory system. The following description is based on known research conclusions and data available in handbooks on the experimental psychology of hearing as presented in the discussion in U.S. patent application Ser. No. 10/145,113, incorporated by reference above.

The human auditory system is overwhelmingly complex, both in design and in function. It comprises thousands of receptors connected by complex neural networks to the auditory cortex in the brain. Different components of incident sound excite different receptors, which in turn channel information towards the auditory cortex through different neural network routes.

The response of an individual receptor to a sound component is not always the same; it depends on various factors such as the spectral make up of the sound signal and the preceding sounds, as these receptors can be tuned to respond to different frequencies and intensities.

Masking Principles

Masking is an important and well-researched phenomenon in auditory perception. It is defined as the amount (or the process) by which the threshold of audibility for one sound is raised by the presence of another (masking) sound. The principles of masking are based upon the way the ear performs spectral analysis. A frequency-to-place transformation takes place in the inner ear, along the basilar membrane. Distinct regions in the cochlea, each with a set of neural receptors, are tuned to different frequency bands, which are called critical bands. The spectrum of human audition can be divided into several critical bands, which are not equal.

In simultaneous masking the masker and the target sounds coexist. The target sound specifies the critical band. The auditory system “suspects” there is a sound in that region and tries to detect it. If the masker is sufficiently wide and loud the target sound cannot be heard. This phenomenon can be explained in simple terms, on the basis that the presence of a strong noise or tone masker creates an excitation of sufficient strength on the basilar membrane at the critical band location of the inner ear effectively to block the transmission of the weaker signal.

For an average listener, the critical bandwidth can be approximated by:

$${BW}_{c} ( f ) = 25 + {75 [ 1 + 1.4 \cdot {( \frac{f}{1000} )}^{2} ]}^{0.69} ( Hz )$$

where BW_{c} is the critical bandwidth in Hz and f the frequency in Hz.

Also, Bark is associated with frequency f via the following equations:

$$Bark = \frac{f}{100} , f > 500 Hz$$

$$Bark = 9 + 4 \cdot {log}_{2} \frac{f}{100} , f > 500 Hz$$

A masker sound within a critical band has some predictable effect on the perceived detection of sounds in other critical bands. This effect, also known as the spread of masking, can be approximated by a triangular function, which has slopes of +25 and −10 dB per bark (distance of 1 critical band), as shown in accompanying FIG. 23.

Principles of the Perceptual Organisation of Sound

The auditory system performs a complex task; sound pressure waves originating from a multiplicity of sources around the listener fuse into a single pressure variation before they enter the ear; in order to form a realistic picture of the surrounding events the listener's auditory system must break down this signal to its constituent parts so that each sound-producing event is identified. This process is based on cues, pieces of information which help the auditory system assign different parts of the signal to different sources, in a process called grouping or auditory object formation. In a complex sound environment there are a number of different cues, which aid listeners to make sense of what they hear.

These cues can be auditory and/or visual or they can be based on knowledge or previous experience. Auditory cues relate to the spectral and temporal characteristics of the blending signals. Different simultaneous sound sources can be distinguished, for example, if their spectral qualities and intensity characteristics, or if their periodicities are different. Visual cues, depending on visual evidence from the sound sources, can also affect the perception of sound.

Auditory scene analysis is a process in which the auditory system takes the mixture of sound that it derives from a complex natural environment and sorts it into packages of acoustic evidence, each probably arising from a single source of sound. It appears that our auditory system works in two ways, by the use of primitive processes of auditory grouping and by governing the listening process by schemas that incorporate our knowledge of familiar sounds.

The primitive process of grouping seems to employ a strategy of first breaking down the incoming array of energy to perform a large number of separate analyses. These are local to particular moments of time and particular frequency regions in the acoustic spectrum. Each region is described in terms of its intensity, its fluctuation pattern, the direction of frequency transitions in it, an estimate of where the sound is coming from in space and perhaps other features. After these numerous separate analyses have been done, the auditory system has the problem of deciding how to group the results so that each group is derived from the same environmental event or sound source.

The grouping has to be done in two dimensions at the least: across the spectrum (simultaneous integration or organization) and across time (temporal grouping or sequential integration). The former, which can also be referred to as spectral integration or fusion, is concerned with the organization of simultaneous components of the complex spectrum into groups, each arising from a single source. The latter (temporal grouping or sequential organization) follows those components in time and groups them into perceptual streams, each arising from a single source again. Only by putting together the right set of frequency components over time can the identity of the different simultaneous signals be recognized.

The primitive process of grouping works in tandem with schema-based organization, which takes into account past learning and experiences as well as attention, and which is therefore linked to higher order processes. Primitive segregation employs neither past learning nor voluntary attention. The relations it creates tend to be valid clues over wide classes of acoustic events. By contrast, schemas relate to particular classes of sounds. They supplement the general knowledge that is packaged in the innate heuristics by using specific learned knowledge.

A number of auditory phenomena have been related to the grouping of sounds into auditory streams, including in particular those related to speech perception, the perception of the order and other temporal properties of sound sequences, the combining of evidence from the two ears, the detection of patterns embedded in other sounds, the perception of simultaneous “layers” of sounds (e.g., in music), the perceived continuity of sounds through interrupting noise, perceived timbre and rhythm, and the perception of tonal sequences.

Spectral integration is pertinent to the grouping of simultaneous components in a sound mixture, so that they are treated as arising from the same source. The auditory system looks for correlations or correspondences among parts of the spectrum, which would be unlikely to have occurred by chance. Certain types of relations between simultaneous components can be used as clues for grouping them together. The effect of this grouping is to allow global analyses of factors such as pitch, timbre, loudness, and even spatial origin to be performed on a set of sensory evidence coming from the same environmental event.

Many of the factors that favor the grouping of a sequence of auditory inputs are features that define the similarity and continuity of successive sounds. These include fundamental frequency, temporal proximity, shape of spectrum, intensity, and apparent spatial origin. These characteristics affect the sequential aspect of scene analysis, in other words the use of the temporal structure of sound.

Generally, it appears that the stream forming process follows principles analogous to the principle of grouping by proximity. High tones tend to group with other high tones if they are adequately close in time. In the case of continuous sounds it appears that there is a unit forming process that is sensitive to the discontinuities in sound, particularly to sudden rises in intensity, and that creates unit boundaries when such discontinuities occur. Units can occur in different time scales and smaller units can be embedded in larger ones.

In complex tones, where there are many frequency components, the situation is more complicated as the auditory system estimates the fundamental frequency of the set of harmonics present in sound in order to determine the pitch. The perceptual grouping is affected by the difference in fundamental frequency pitch) and/or by the difference in the average of partials (brightness) in a sound. They both affect the perceptual grouping and the effects are additive.

A pure tone has a different spectral content than a complex tone; so, even if the pitches of the two sounds are the same, the tones will tend to segregate into different groups from one another. However another type of grouping may take effect: a pure tone may, instead of grouping with the entire complex tone following it, group with one of the frequency components of the latter.

Location in space may be another effective similarity, which influences temporal grouping of tones. Primitive scene analysis tends to group sounds that come from the same point in space and segregate those that come from different places. Frequency separation, rate, and the spatial separation combine to influence segregation. Spatial differences seem to have their strongest effect on segregation when they are combined with other differences between the sounds.

In a complex auditory environment where distracting sounds may come from any direction on the horizontal plane, localization seems to be very important, as disrupting the localization of distracting sound sources can weaken the identity of particular streams.

Timbre is another factor that affects the similarity of tones and hence their grouping into streams. The difficulty is that timbre is not a simple one-dimensional property of sounds. One distinct dimension however is brightness. Bright tones have more of their energy concentrated towards high frequencies than dull tones do, since brightness is measured by the mean frequency obtained when all the frequency components are weighted according to their loudness. Sounds with similar brightness will tend to be assigned to the same stream. Timbre is a quality of sound that can be changed in two ways: first by offering synthetic sound components to the mixture, which will fuse with the existing components; and second by capturing components out of a mixture by offering them better components with which to group.

Generally speaking, the pattern of peaks and valleys in the spectra of sounds affects their grouping. However there are two types of spectra similarity, when two tones have their harmonics peaking at exactly the same frequencies and when corresponding harmonics are of proportional intensity (if the fundamental frequency of the second tone is double that of the first, then all the peaks in the spectrum would be at double the frequency). Available evidence has shown that both forms of spectra similarity are used in auditory scene analysis to group successive tones.

Continuous sounds seem to hold better as a single stream than discontinuous sounds do. This occurs because the auditory system tends to assume that any sequence that exhibits acoustic continuity has probably arisen from one environmental event.

Competition between different factors results in different organizations; it appears that frequency proximities are competitive and that the system tries to form streams by grouping the elements that bear the greatest resemblance to one another. Because of the competition, an element can be captured out of a sequential grouping by giving it a better sound to group with.

The competition also occurs between different factors that favor grouping. For example in a four tone sequence ABXY if similarity in fundamental frequencies favors the groupings AB and XY, while similarity in spectral peaks favors the grouping AX and BY, then the actual grouping will depend on the relative sizes of the differences.

There is also collaboration as well as competition. If a number of factors all favor the grouping of sounds in the same way, the grouping will be very strong, and the sounds will always be heard as parts of the same stream. The process of collaboration and competition is easy to conceptualize. It is as if each acoustic dimension could vote for a grouping, with the number of votes cast being determined by the degree of similarity with that dimension and by the importance of that dimension. Then streams would be formed, whose elements were grouped by the most votes. Such a voting system is valuable in evaluating a natural environment, in which it is not guaranteed that sounds resembling one another in only one or two ways will always have arisen from the same acoustic source.

Primitive processes of scene analysis are assumed to establish basic groupings amongst the sensory evidence, so that the number and the qualities of the sounds that are ultimately perceived are based on these groupings. These groupings are based on rules which take advantage of fairly constant properties of the acoustic world, such as the fact that most sounds tend to be continuous, to change location slowly and to have components that start and end together. However, auditory organization would not be complete if it ended there. The experiences of the listener are also structured by more refined knowledge of particular classes of signals, such as speech, music, animal sounds, machine noises and other familiar sounds of our environment.

This knowledge is captured in units of mental control called schemas. Each schema incorporates information about a particular regularity in our environment. Regularity can occur at different levels of size and spans of time. So, in our knowledge of language we would have one schema for the sound “a”, another for the word “apple”, one for the grammatical structure of a passive sentence, one for the give and take pattern in a conversation and so on.

It is believed that schemas become active when they detect, in the incoming sense data, the particular data that they deal with. Because many of the patterns that schemas look for extend over time, when part of the evidence is present and the schema is activated, it can prepare the perceptual process for the remainder of the pattern. This process is very important for auditory perception, especially for complex or repeated signals like speech. It can be argued that schemas, in the process of making sense of grouped sounds, occupy significant processing power in the brain. This could be one explanation for the distracting strength of intruding speech, a case where schemas are involuntarily activated to process the incoming signal. Limiting the activation of these schemas either by affecting the primitive groupings, which activate them or by activating other competing schemas less “computationally expensive” for the brain reduces distractions.

There are cases in which primitive grouping processes seem not to be responsible for the perceptual groupings. In these cases schemas select evidence that has not been subdivided by primitive analysis. There are also examples that show another capacity: the ability to regroup evidence that has already been grouped by primitive processes.

Our voluntary attention employs schemas as well. For example, when we are listening carefully for our name being called out among many others in a list we are employing the schema for our name. Anything that is being listened for is part of a schema, and thus whenever attention is accomplishing a task, schemas are participating.

It will be appreciated from the above that the human auditory system is closely attuned to its environment, and unwanted sound or noise has been recognized as a major problem in industrial, office and domestic environments for many years now. Advances in materials technology have provided some solutions. However, the solutions have all addressed the problem in the same way, namely: the sound environment has been improved either by decreasing or by masking noise levels in a controlled space.

Conventional masking systems generally rely on decreasing the signal to noise ratio of distracting sound signals in the environment, by raising the level of the prevailing background sound. A constant component, both in frequency content and amplitude, is introduced into the environment so that peaks in a signal, such as speech, produce a low signal to noise ratio. There is a limitation on the amplitude level of such a steady contribution, defined by the user acceptance: a level of noise that would mask even the higher intruding speech signals would probably be unbearable for prolonged periods. Furthermore this component needs to be wide enough spectrally to cover most possible distracting sounds.

In addition, known masking systems are either systems installed centrally in a space permitting the users of the space very limited or no control over their output, or are self-contained systems with limited inputs, if any, that permit only one user situated adjacent to the masking system control of a small number of system parameters.

Accordingly, it is desirable to provide a more flexible system for, and method of, acoustically improving an environment. Such a system based on the principles of human auditory perception described above provide a reactive system capable of inhibiting and/or prohibiting the effective communication of sound that is perceived as noise by means of an output which is variably dependent on the noise. One feature of such a system includes the ability to provide manual adjustment by one or more users using a simple graphical user interface. These users may be local to such a system or remote from it. Another feature of such a flexible system may include automatic adjustment of parameters once the user initially conditions the system parameters. Adjustment of a large number of parameters of such a system, while perhaps increasing the number of inputs, also correspondingly would allow the user to tailor the sound environment of the occupied space to his or her specific preferences.

### BRIEF SUMMARY

By way of introduction only, in one embodiment an electronic sound screening system contains a receiver, a converter, an analyser, a processor and a sound generator. Acoustic energy impinges on the receiver and is converted to an electrical signal by the converter. The analyser receives the electrical signal from the receiver, analyzes the electrical signal, and generates data analysis signals in response to the analyzed electrical signal. The processor produces sound signals based on the data analysis signals from the analyser in each of a plurality of frequency bands which correspond to the critical bands of the human auditory system (also known as Bark Scale ranges). The sound generator provides sound based on the sound signals.

In another embodiment, the electronic sound screening system contains a controller that is manually settable that provides user signals based on user selected inputs in addition to the receiver, the converter, the analyser, a processor and the sound generator. In this case, the processor produces sound signals and contains a harmonic brain that forms a harmonic base and system beat. The sound signals are selectable from dependent signals that are set to be dependent upon the received acoustic energy (produced by certain modules within the processor) and independent signals that are set to be independent of the received acoustic energy (produced by other modules within the processor). These modules include, for example, mask the sound functionally and/or harmonically, filter the signals, produce chords, motives and/or arpeggios, control signals and/or use prerecorded sounds.

In another embodiment, the sound signals produced by the processor are selectable from processing signals that are generated by direct processing of the data analysis signals, generative signals that are generated algorithmically and are adjusted by data analysis signals or scripted signals that are predetermined by a user and are adjusted by the data analysis signals.

In another embodiment, in addition to the receiver, the converter, the analyser, a processor and the sound generator, the sound screening system contains a local user interface through which a local user enters local user inputs to change a state of the sound screening system and a remote user interface through which a non-local user enters remote user inputs to change the state of the sound screening system. The interface, such as a web browser, allows one or more users to affect characteristics of the sound screening system. For example, users vote on a particular characteristic or parameter of the sound screening system, the votes are given different weights (in accordance with the distance of the user from the sound screening system for instance) and then averaged to produce the final result that determines how the sound screening system behaves. Local users may be, for example, in the immediate vicinity of the sound screening system while remote users may be farther away. Alternatively, local users can be, say, within a few feet while remote users can be, say, more than about ten feet from the sound screening system. Obviously, these distances are merely exemplary.

In another embodiment, in addition to the receiver, the converter, the analyser, a processor and the sound generator, the sound screening system contains a communication interface through which multiple systems can establish bi-directional communication and exchange signals for synchronizing their sound analysis and response processes and/or for sharing analysis and generative data, thus effectively establishing a sound screening system of larger physical scale.

In another embodiment, the sound screening system employs a physical sound attenuating screen or boundary on which sound sensing and sound emitting components are placed in such a way that they effectively operate primarily on the side of the screen or boundary on which they are positioned and a control system through which a user can select the side of the screen or boundary on which input sound will be sensed and the side of the screen or boundary on which sound will be emitted.

In different embodiments, the sound screening system is operated through computer-executable instructions in any computer readable medium that controls the receiver, the converter, the analyser, a processor, the sound generator and/or the controller.

The foregoing summary has been provided only by way of introduction. Nothing in this section should be taken as a limitation on the following claims, which define the scope of the invention.

### BRIEF DESCRIPTION OF THE DRAWINGS

FIG. 1 is a general schematic diagram illustrating the operation of the sound screening system.

FIG. 2 illustrates an embodiment of the sound screening system of FIG. 1.

FIG. 3 shows a detailed view of the sound screening algorithm of FIG. 3

FIG. 4 is an embodiment of the System Input of FIG. 3.

FIG. 5 is an embodiment of the Analyser of FIG. 3.

FIG. 6 is an embodiment of the Analyser History of FIG. 3.

FIG. 7 is an embodiment of the Harmonic Brain of FIG. 3.

FIG. 8 is an embodiment of the Functional Masker of FIG. 3.

FIG. 9 is an embodiment of the Harmonic Masker of FIG. 3.

FIG. 10 is an embodiment of the Harmonic Voiceset of FIG. 9.

FIG. 11 is an embodiment of the Chordal and Arpeggiation soundsprites of FIG. 3.

FIG. 12 is an embodiment of the Motive soundsprite of FIG. 3.

FIG. 13 is an embodiment of the Cloud soundsprite of FIG. 3.

FIG. 14 is an embodiment of the Control soundsprite of FIG. 3.

FIG. 15 is an embodiment of a Chord Generator soundsprite of FIG. 9.

FIG. 16 shows a view per parameter type of the sound screening algorithm of FIG. 3.

FIG. 17 shows a view of the main routine section of the GUI of the sound screening algorithm of FIG. 3.

FIG. 18 shows a System Input window of the main routine section of FIG. 17.

FIG. 19 shows an Analyser window of the main routine section of FIG. 17.

FIG. 20 shows an Analyser History window of the main routine section of FIG. 17.

FIG. 21 shows a Soundscape Base window of the main routine section of FIG. 17.

FIG. 22 shows Global Harmonic Progression and a Masterchords settings table of the Soundscape Base of FIG. 21.

FIG. 23 shows a Functional Masker window of the main routine section of FIG. 17.

FIG. 24 shows a Harmonic Masker window of the main routine section of FIG. 17.

FIG. 25 shows a Chordal soundsprite window of the main routine section of FIG. 17.

FIG. 26 shows an Arpeggio soundsprite window of the main routine section of FIG. 17.

FIG. 27 shows a Motive soundsprite window of the main routine section of FIG. 17.

FIG. 28 shows a Clouds soundsprite window of the main routine section of FIG. 17.

FIG. 29 shows a Control soundsprite window of the main routine section of FIG. 17.

FIG. 30 shows a Soundfile soundsprite window of the main routine section of FIG. 17.

FIG. 31 shows a Solid Filter soundsprite window of the main routine section of FIG. 17.

FIG. 32 shows a Control soundsprite window of the main routine section of FIG. 17.

FIG. 33 shows a Synth Effects window of the main routine section of FIG. 17.

FIG. 34 shows a Mixer window of FIG. 17.

FIG. 35 shows a Preset Selector Panel window of FIG. 17.

FIG. 36 shows a Preset Calendar window of FIG. 17.

FIG. 37 shows a Preset Selection Dialog Box window of FIG. 17.

FIG. 38 shows the intercom receive channels in an Arpeggio generation window.

FIG. 39 shows the intercom parameter processing in the Arpeggio generation window of FIG. 38.

FIG. 40 shows the intercom connect to channels in the Arpeggio generation window of FIG. 38.

FIG. 41 shows the intercom broadcast section prior to setup in the Arpeggio generation window of FIG. 38.

FIG. 42 shows the intercom parameter broadcast menu in the Arpeggio generation window of FIG. 38.

FIG. 43 shows the intercom broadcast channel menu in the Arpeggio generation window of FIG. 38.

FIG. 44 shows the intercom broadcast section after setup in the Arpeggio generation window of FIG. 38.

FIG. 45 shows the intercom connections display menu of FIG. 17.

FIG. 46 shows a LAN control system of the GUI.

FIG. 47 shows a further view of the LAN control system of FIG. 31.

FIG. 48 shows a further view of the LAN control system of FIG. 31

FIG. 49 shows a schematic of the system employing various input and output components

FIG. 50. shows an embodiment for the speaker subassembly employed in FIG. 49

FIG. 51. shows a further view of the speaker subassembly of FIG. 50

FIG. 52. shows an workgroup sound screening system

FIG. 53. shown an architectural sound screening system

### DETAILED DESCRIPTION OF THE PRESENTLY PREFERRED EMBODIMENTS

The present sound screening system is a highly flexible system using specially designed software architecture containing a number of modules that receive and analyze environmental sound on the one hand and produce sound in real time or near real time on the other. The software architecture and modules provide a platform in which all sound generation subroutines (for easier referencing, all sound producing subroutines—tonal, noise based or otherwise—are referenced as soundsprites) are connected with the rest of the system and to each other. This ensures forward compatibility with soundsprites that might be developed in the future or even soundsprites from independent developers.

Multiple system inputs are also provided. These inputs include user inputs and input analysis data adjusted through mapping. The mapping uses an intercom system that broadcasts specific changing parameters along a particular channel. The channels are received by the various modules within the sound screening system and information is transported along the channels used to control various aspects of the sound screening system. This allows the software architecture and modules to provide a flexible architecture for the sharing of parameters within various parts of the system, to enable, for example, any soundsprite to be responsive to any input analysis data if required, or to any parameter generated from other soundsprites.

The system permits both local and remote control. Local control is control effected in the local environ of the sound screening system, for example, in a workstation within which the sound screening system is disposed or within a few feet of the sound screening system. If one or more remote users desire to control the sound screening system, they are permitted weighed voting as to the user settings commensurate with their location from the sound screening system and/or other variables.

The sound screening system encompasses a specific communication interface enabling multiple systems to communicate with each other and establish a sound screening system of a larger scale, for example covering floor plans of several hundred square feet.

Furthermore, the sound screening system described in the invention uses multiple sound receiving units, for example microphones, and multiple sound emitting units, for example speakers, which may be distributed in space, or positioned on either side of a sound attenuating screen and permits user control as to which combination of sound receiving and sound emitting sources will be active at any one time.

The sound screening system may contain a physical sound screen which may be a wall or screen that is self-contained or housed within another receptacle, for example, as shown and described in the applications incorporated by reference above.

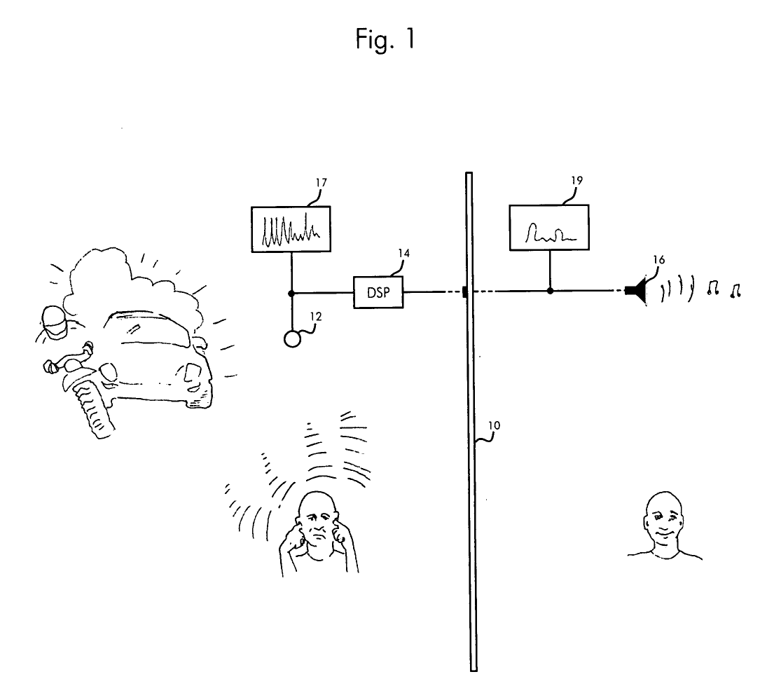

FIG. 1 illustrates a system for acoustically improving an environment in a general schematic diagram, which includes a partitioning device in the form of a curtain 10. The system also comprises a number of microphones 12, which may be positioned at a distance from the curtain 10 or which may be mounted on, or integrally formed in, a surface of the curtain 10. The microphones 12 are electrically connected to a digital signal processor (DSP) 14 and thence to a number of loudspeakers 16, which again may be positioned at a distance from the curtain or mounted on, or integrally formed in, a surface of the curtain 10. The curtain 10 produces a discontinuity in a sound conducting medium, such as air, and acts primarily as a sound absorbing and/or reflecting device.

The microphones 12 receive ambient noise from the surrounding environment and convert such noise into electrical signals for supply to the DSP 14. A spectrogram 17 representing such noise is illustrated in FIG. 1. The DSP 14 employs an algorithm firstly for performing an analysis of such electrical signals to generate data analysis signals, and thence in response to such data analysis signals for producing sound signals for supply to the loudspeakers 16. A spectrogram 19 representing such sound signals is illustrated in FIG. 1. The sound issuing from the loudspeakers 16 may be an acoustic signal based on the analysis of the original ambient noise, for example from which certain frequencies have been selected to generate sounds having a pleasing quality to the user(s).

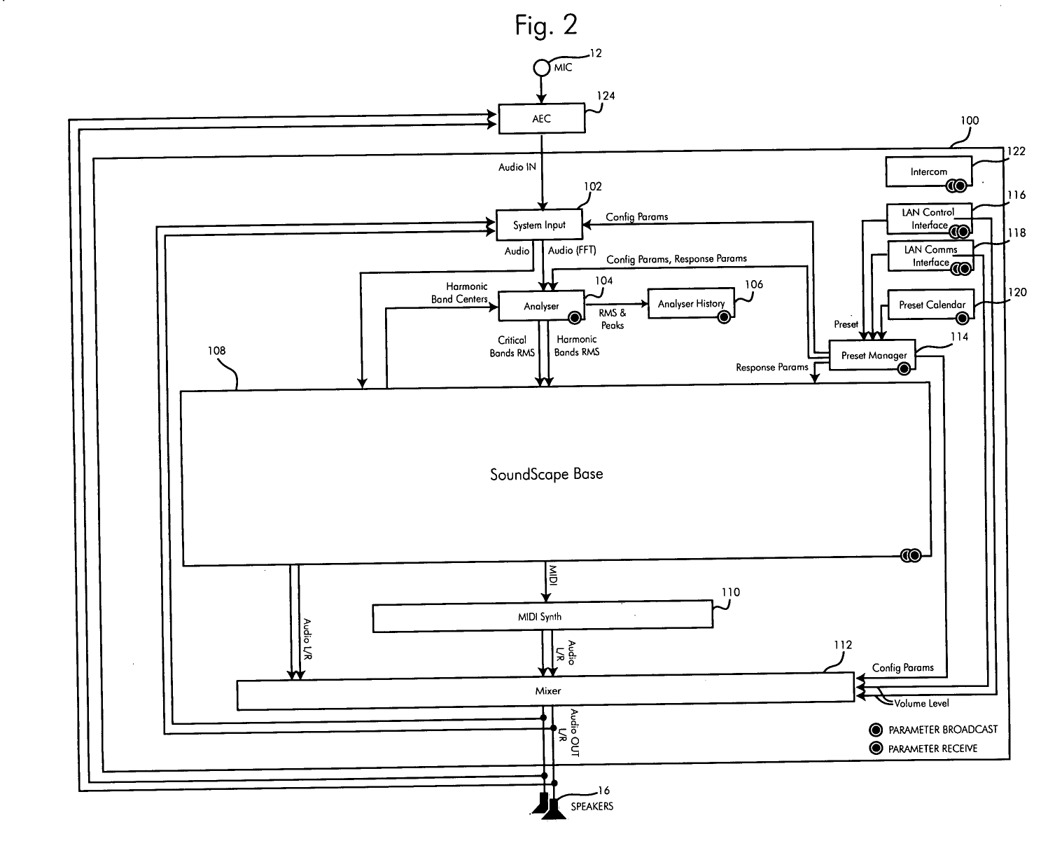

The DSP 14 serves to analyse the electrical signals supplied from the microphones 12 and in response to such analysed signals to generate sound signals for driving the loudspeakers 16. For this purpose, the DSP 14 employs an algorithm, described below with reference to FIGS. 2 to 32.

FIG. 2 illustrates one embodiment of the sound screening algorithm 100, with paths along which information flows. The sound screening algorithm 100 contains a system input 102 that receives acoustic energy from the environment and translates it into input signals using a fast-Fourier transform (FFT). The FFT signals are fed to an Analyser 104, which then analyzes the FFT signals in a manner similar to but more closely attuned to the human auditory system than the Interpreter in the applications incorporated by reference. The analysed signals are then stored in a memory called the Analyser History 106. The Analyser 104, among other things, calculates peak and root-mean-square (RMS, or, energy) values of the signals in the various critical bands, as well as those in the harmonic bands. These analyzed signals are transmitted to a Soundscape Base 108, which incorporates all of the soundsprites and thus generates one or more patterns in response to the analyzed signals. The Soundscape Base 108, in turn, supplies the Analyser 104 with information the Analyser 104 uses to analyze the FFT signals. Use of the Soundscape Base 108 allows elimination of the distinction between masker and tonal engine in previous embodiments of the sound screening system.

The Soundscape Base 108 additionally outputs MIDI signals to a MIDI Synthesizer 110 and audio left/right signals to a Mixer 112. The Mixer 112 receives signals from the MIDI Synthesizer 110, a Preset Manager 114, a Local Area Network (LAN) controller 116, and a LAN communicator 118. The Preset Manager 114 also supplies signals to the Soundscape Base 108, the Analyser 104 and the System Input 102. The Preset Manager 114 receives information from the LAN controller 116, LAN communicator 118, and a Preset Calendar 120. The output of the Mixer 112 is fed to speakers 16 as well as used as feedback to the System Input 102 on the one hand and to the Acoustic Echo Canceller 124 on the other.

The signals between the various modules, including those transmitted using channels on the Intercom 122 as well as between local and remote systems, may be transmitted through wired or wireless communication. For example, the embodiment shown permits synchronized operation of multiple reactive sound systems, which may be in physical proximity to each other or not. The LAN communicator 118 handles the interfacing between the local system and remote systems. Additionally, the present system provides the capability for user tuning over a local area network. The LAN Control 116 handles the data exchange between the local system and a specially built control interface accessible via an Internet browser by any user with access privileges. As above, other communication systems can be used, such as wireless systems using Bluetooth protocols.

Internally, as shown only some of the modules can transmit or receive over the Intercom 122. More specifically, the System Input 102, the MIDI Synthesizer 110 and the Mixer 112 are not adjusted by the changing parameters and thus do not make use of the Intercom 122. Meanwhile, the Analyser 104 and Analyser History 106 broadcast various parameters through the Intercom 122 but do not receive parameters to generate the analyzed or stored signals.

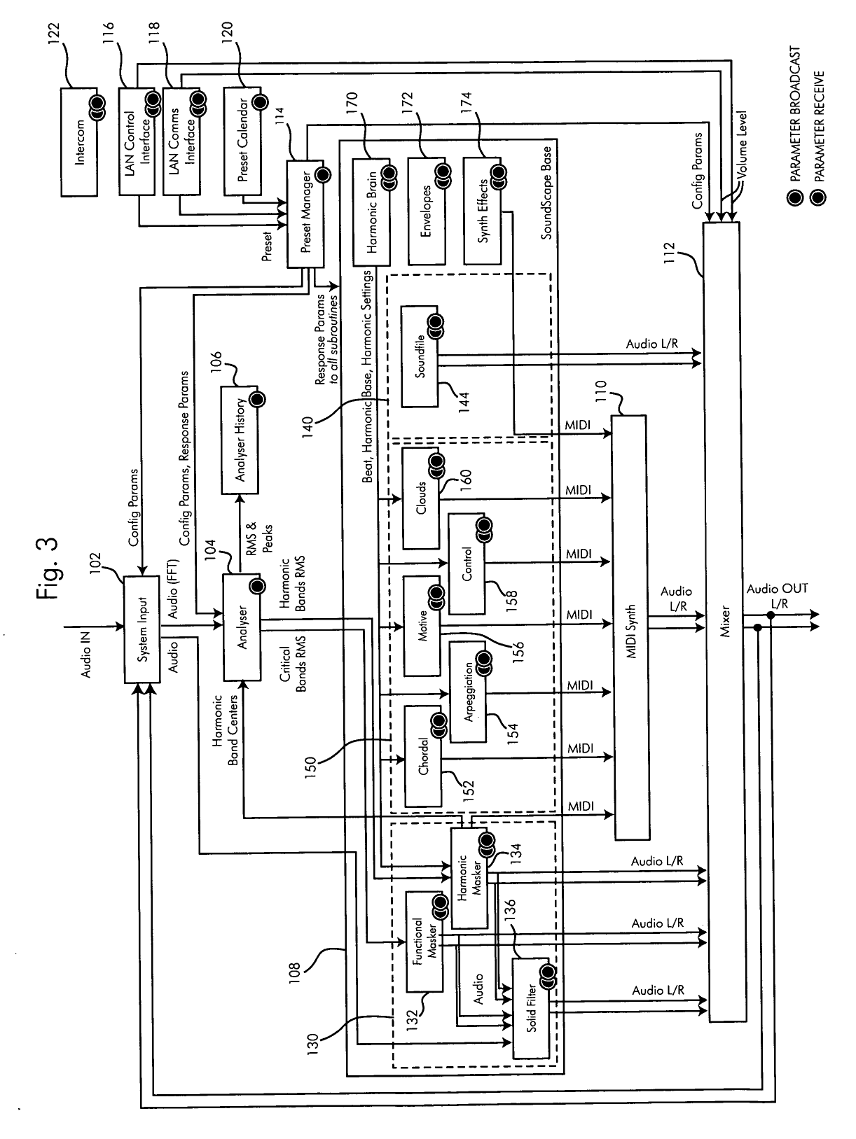

The Preset Manager 114, the Preset Calendar 120, the LAN controller 116 and LAN communicator 118, as well as some of the soundsprites in the Soundscape Base 108, as shown in FIG. 3, broadcast and/or receive parameters through the Intercom 122.

As FIG. 3 is essentially the same as FIG. 2, with soundsprites disposed within the Soundscape Base 108 shown, elements other than the Soundscape Base 108 will not be labeled. In FIG. 3, only soundsprites that provide different outputs disposed within the Soundscape Base 108 are shown. This is to say multiple soundsprites that have similar outputs may be present, as illustrated in the GUI figures below; thus, different soundsprites may have similar outputs (e.g. two Arpeggiation soundsprites 154 that are affected by parameters received in one or more channels differently) or different outputs (e.g. an Arpeggiation soundsprite 154 and Chordal soundsprite 152).

The Soundscape Base 108 is similar to the Tonal Engine and Masker of the applications incorporated by reference, but has a number of different types of soundsprites. The Soundscape Base 108 contains soundsprites that are broken up into three categories: electroacoustic soundsprites are generated by direct processing of the sensed input 130, scripted soundsprites 140 that are predetermined note sequences or audio files that are conditioned by the sensed input, and generative soundsprites 150 that are generated algorithmically or conditioned by the sensed input. The electroacoustic soundsprites 130 produce sound based on the direct processing of the analyzed signals from the Analyser 104 and/or the audio signal from the System Input 102; the remaining soundsprites produce sound generatively by employing user input but can have their output adjusted or conditioned by the analysed signals from the Analyser 104. Each of the soundsprites is able to communicate using the Intercom 122, with all of the soundsprites being able to broadcast and receive parameters to and from the intercom. Similarly, each of the soundsprites is able to be affected by the Preset Manager.

Each of the generative soundsprites 150 produce MIDI signals that are transmitted to the Mixer 112 through the MIDI Synthesizer 110, and each of the electroacoustic soundsprites 130 produce audio signals that are transmitted to the Mixer 112 directly without going through the MIDI Synthesizer 110 or audio signals that are transmitted to the Mixer 112 directly, in addition to producing MIDI signals that are transmitted to the Mixer 112 through the MIDI Synthesizer 110. The scripted soundsprites 140 produce audio signals, but can also be programmed to produce pre-described MIDI sequences transmitted to the Mixer 112 through the MIDI Synthesizer 110.

In addition to the various soundsprites, the Soundscape Base 108 also contains a Harmonic Brain 170, Envelope 172 and Synth Effects 174. The Harmonic Brain 170 provides the beat, the harmonic base, and the harmonic settings to those soundsprites that use such information in generating an output signal. The Envelope 172 provides streams of numerical values that change with a pre-described manner, as input by the user, over a length of time, also input by the user. The Synth FX 174 soundsprite sets the preset of the MIDI Synthesizer 110 effects channel, which is used as the global effects settings for all the outputs of the MIDI Synth 110.

The electroacoustic soundsprites 130 include a functional masker 132, a harmonic masker 134, and a solid filter 136. The scripted soundsprites 140 include a soundfile 144. The generative soundsprites 150 include Chordal 152, Arpeggiation 154, Motive 156, Control 158, and Clouds 160.

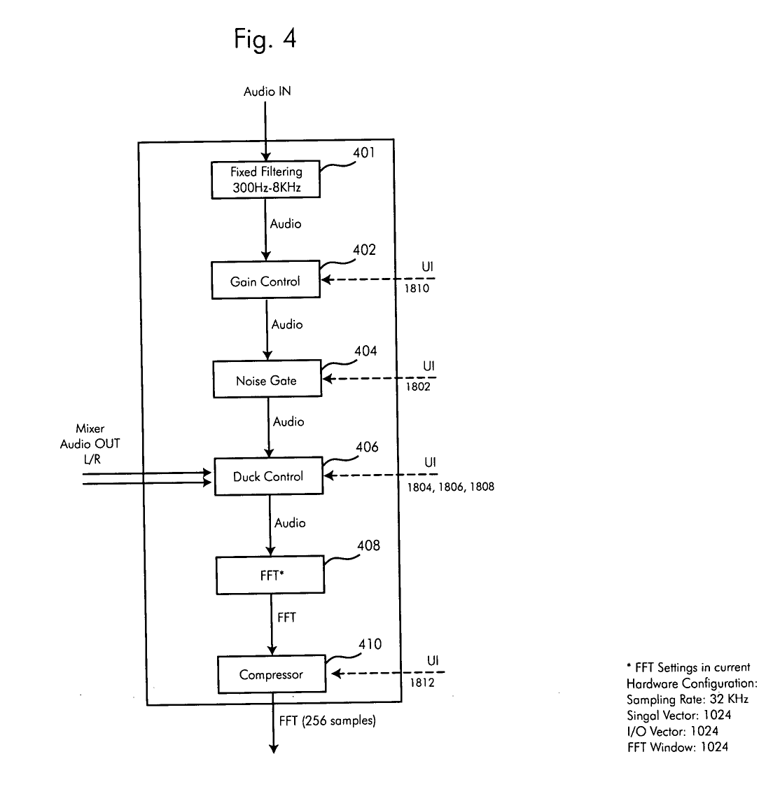

The System Input 400 will now be described in more detail, with reference to FIG. 4. As shown, the System Input 400 contains several sub-modules. As illustrated, the System Input 400 contains a sub-module to filter the audio signals supplied to the input. The Fixed Filtering sub-module 401 contains one or more filters. As shown, these filters pass input signals between 300 Hz and 8 kHz. The filtered audio signal then is provided to an input of a Gain Control sub-module 402. The Gain Control sub-module 402 receives the filtered audio signal and provides a multiplied audio signal to an output thereof. The multiplied audio signal is multiplied by gain factor determined by an externally applied user input (UI) from configuration parameters supplied by the Preset Manager 114.

The multiplied audio signal is then supplied to an input of Noise Gate 404. The Noise Gate 404 acts as a noise filter, supplying the input signal to an output thereof only if it receives a signal higher than a user-defined noise threshold (again referred to as a user input, or UI). This threshold is supplied to the Noise Gate 404 from the Preset Manager 114. The signal from the Noise Gate 404 then is provided to an input of a Duck Control sub-module 406. The Duck Control sub-module 406, essentially acts as an amplitude feedback mechanism that reduces the level of the signal through it when the system output level rises and the sub-module is activated. As shown, the Duck Control sub-module 406 receives the system output signal from the Mixer 112 and is activated by a user input from the Preset Manager 114. The Duck Control sub-module 406 has settings for the amount by which the input signal level is reduced, how quickly the input signal level is reduced (a lower gradient results in lower output), and the time period over which the output level of the Duck Control sub-module 406 is smoothed.

The signal from the Duck Control sub-module 406 is then passed on to an FFT sub-module 408. The FFT sub-module 408 takes the analog signal input thereto and produces a digital output signal of 256 floating-point values representing an FFT frame for a frequency range of 0 to 11,025 Hz. The FFT vectors represent signal strength in evenly distributed bands 31.25 Hz wide for when the FFT analysis is performed at a sampling rate of 32 kHz with full FFT vectors of 1024 values in length. Of course other setting can also be used. No user input is supplied to the FFT sub-module 408. The digital signal from the FFT sub-module 408 is then supplied to a Compressor sub-module 410. The Compressor sub-module 410 acts as an automatic gain control that supplies the input digital signal as the output signal from the Compressor sub-module 410 when the input signal is lower than a compressor threshold level and multiplies the input digital signal by a factor smaller than 1 (i.e. reduces the input signal) when the input signal is higher than the threshold level to provide the output signal. The compressor threshold level of the Compressor sub-module 410 is supplied as a user input from the Preset Manager 114. If the multiplication factor is set to zero, the level of the output signal is effectively limited to the compressor threshold level. The output signal from the Compressor sub-module 410 is the output signal from the System Input 400. Thus, an analog signal is supplied to an input of the System Input 400 and a digital signal is supplied from an output of the System Input 400.

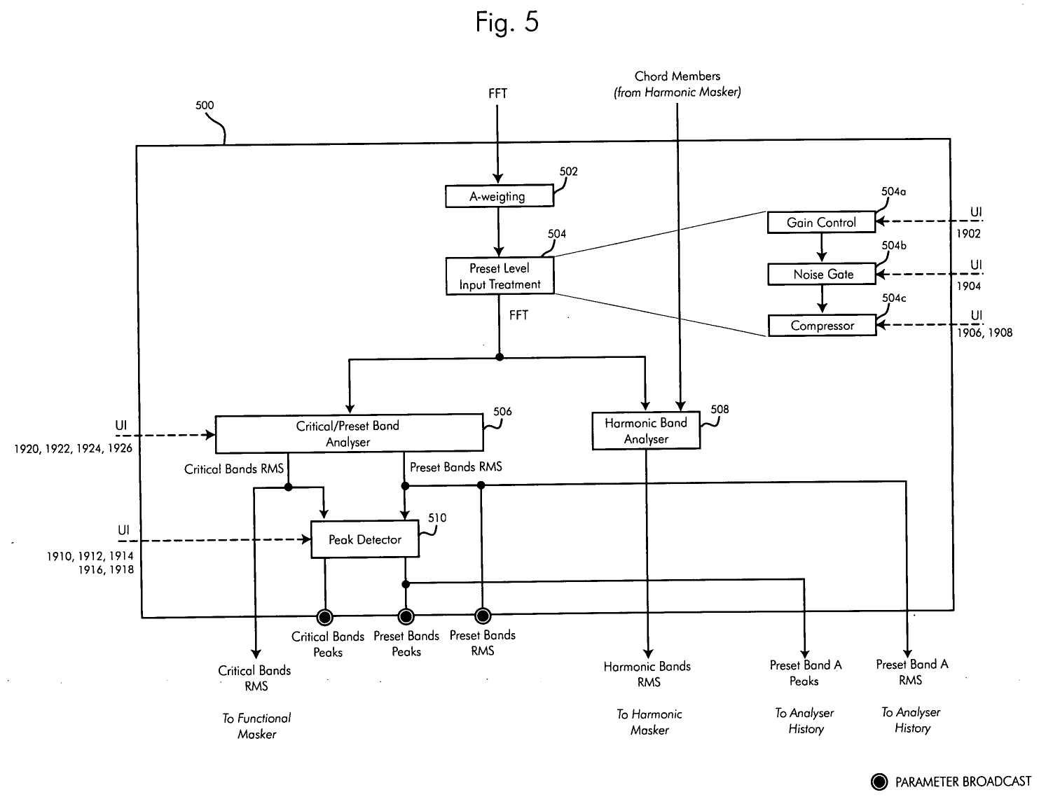

The digital FFT output signal from the System Input 400 is supplied to the Analyser 500, along with configuration parameters from the Preset Manager 114 and chords from the Harmonic Masker 134, as shown in FIG. 5. The Analyser 500 also has a number of sub-modules. The FFT input signal is supplied to an A-weighting sub-module 502. The A-weighting sub-module 502 adjusts the frequencies of the input FFT signal to take account of the non-linearity of the human auditory system.

The output from the A-weighting sub-module 502 is then supplied to a Preset Level Input Treatment sub-module 504, which contains sub-sub-modules that are similar to some of the modules in the System Input 400. The Preset Level Input Treatment sub-module 504 contains a Gain Control sub-sub-module 504a, a Noise Gate sub-sub-module 504b, and a Compressor sub-sub-module 504c. Each of these sub-sub-modules have similar user input parameters supplied from the Preset Manager 114 as those supplied to the corresponding sub-modules in the System Input 400; a gain multiplier is supplied to the Gain Control sub-sub-module 504a, a noise threshold is supplied to the Noise Gate sub-sub-module 504b, and a compressor threshold and compressor multiplier are supplied to Compressor sub-sub-module 504c. The user inputs supplied to the sub-sub modules are saved as Sound/Response Parameters in the Preset Manager 114.

The FFT data from the A-weighting sub-module 502 is then supplied to a Critical/Preset Band Analyser sub-module 506 and a Harmonic Band Analyser sub-module 508. The Critical/Preset Band Analyser sub-module 506 accepts the incoming FFT vectors representing A-weighted signal strength in 256 evenly distributed bands and aggregates the spectrum values into 25 critical bands on the one hand and into 4 preset selected frequency Bands on the other hand, using a Root Mean Square function. The frequency boundaries of the 25 critical bands are fixed and dictated by auditory theory. Table 1 shows the frequency boundaries uses in this embodiment, but different definitions of the critical bands, following different auditory modeling principles can also be used. The frequency boundaries of the 4 preset selected frequency bands are variable upon user control and are advantageously selected such that they provide useful analysis data for the particular sound environment in which the system might be installed. The preset selected bands are set to contain a combination of entire critical bands, from a single critical band to any combination of all 25 critical bands. Although only four preset selected bands are indicated in FIG. 5, a greater or lesser number of bands may be selected.

The Critical/Preset Band Analyser sub-module 506 receives detection parameters from the Preset Manager 114. These detection parameters include definitions of the four frequency ranges for the preset selected frequency bands.

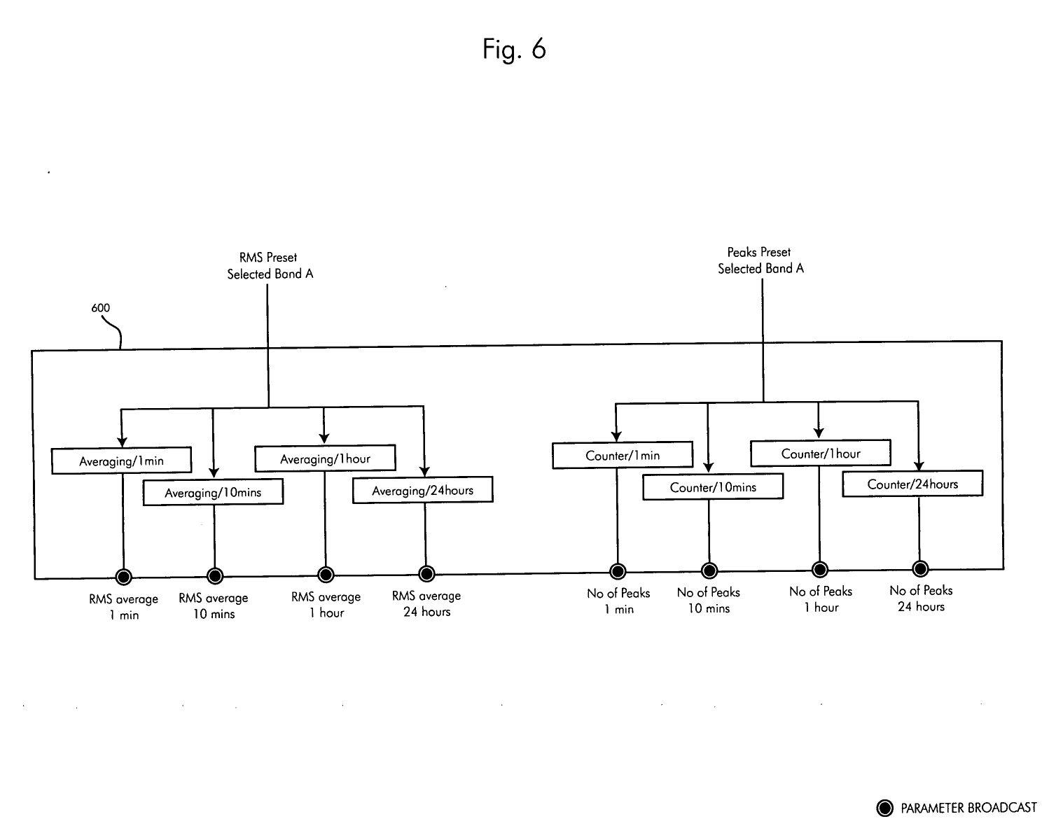

The 25 critical band RMS values produced by the Critical/Preset Band Analyser 506 are passed into the Functional Masker 132 and the Peak Detector 510. This is to say that the Critical/Preset Band Analyser sub-module 506 supplies the RMS values of all of the critical bands (lists of 25 members) to the Functional Masker 132. The 4 preset band RMS values are passed to the Peak Detector 510 and are also broadcast over the Intercom 122. In addition, the RMS values for one of the preset bands are supplied to the Analyzer History 106 (relabeled 600 in FIG. 6).

The Peak Detector sub-module 510 performs windowed peak detection on each of the critical bands and the preset selected bands independently. For each band, a history of signal level is maintained, and this history is analysed by a windowing function. The start of a peak is categorised by a signal contour having a high gradient and then leveling off; the end of a peak is categorised by the signal level dropping to a proportion of its value at the start of the peak.

The Peak Detector sub-module 510 sub-module 506 receives detection parameters from the Preset Manager 114. These detection parameters include definitions for the peak detection and parameters in addition to a parameter defining the duration of a peak event after it has been detected.

The Peak Detector 510 produces Critical Band Peaks and Preset Band Peaks which are broadcast over the Intercom 122. Also Peaks for one of the Preset Bands are passed to the Analyser History Module 106.

TABLE 1
Critical band definition used in sub-module 506

| | Center | Bandwidth |
|---|---|---|
| Band | Frequency (Hz) | (Hz) |
| 1 | 50 | -100 |
| 2 | 150 | 100-200 |
| 3 | 250 | 200-300 |
| 4 | 350 | 300-400 |
| 5 | 450 | 400-510 |
| 6 | 570 | 510-630 |
| 7 | 700 | 630-770 |
| 8 | 840 | 770-920 |
| 9 | 1000 | 920-1080 |
| 10 | 1175 | 1080-1270 |
| 11 | 1370 | 1270-1480 |
| 12 | 1600 | 1480-1720 |
| 13 | 1850 | 1720-2000 |
| 14 | 2150 | 2000-2320 |
| 15 | 2500 | 2320-2700 |
| 16 | 2900 | 2700-3150 |
| 17 | 3400 | 3150-3700 |
| 18 | 4000 | 3700-4400 |
| 19 | 4800 | 4400-5300 |
| 20 | 5800 | 5300-6400 |
| 21 | 7000 | 6400-7700 |
| 22 | 8500 | 7700-9500 |
| 23 | 10,500 | 9500-12000 |
| 24 | 13,500 | 12000-15500 |
| 25 | 19,500 | 15500- |

The Harmonic Band Analyser sub-module 508, which also receives the FFT data from the Preset Level Input Treatment sub-module 504, is supplied with information from the Harmonic Masker 134. The Harmonic Masker 134 provides the band center frequencies that correspond to a chord generated by the Harmonic Masker 134. The Harmonic Band Analyser sub-module 508 supplies the RMS values of the harmonic bands determined by the center frequencies to the Harmonic Masker 134. Again, although only six such bands are indicated in FIG. 5, a greater or lesser number of bands may be selected.

The Analyser History 600 of FIG. 6 receives both the RMS and peak values of one preset selected band corresponding to a single critical band or a set of individual critical bands from the Analyser 500. The RMS values are supplied to various sub-modules that average the RMS values over different periods of time, while the peak values are supplied to various sub-modules that count the number of peaks over different periods of time. As shown, the different periods of time for each of these are 1 minute, 10 minutes, 1 hour, and 24 hours. These periods may be adjusted to any length, as desired, and do not have to be the same between the RMS and peak sub-modules. Also, the Analyser History 500 can be easily modified to receive any number of preset selected or critical bands, if such bands are rendered perceptually important.

The values calculated in the Analyser History 500 are characteristic of the acoustic environment in which an electronic sound screening system is installed. For an appropriately selected preset band, the combination of these values provide a reasonably good signature of the acoustic environment over a period of 24 hrs. This can be a very useful tool for the installation engineer, the acoustic consultant or the sound designer when designing the response of the electronic sound screening system for any particular space; they can recognise the energy and peak patterns characteristic of the space and can design the system output to work with these patterns throughout the day.

The outputs of the Analyser History 500 (each of the RMS averages and peak counts) are broadcast over assigned intercom channels of the Intercom 122.

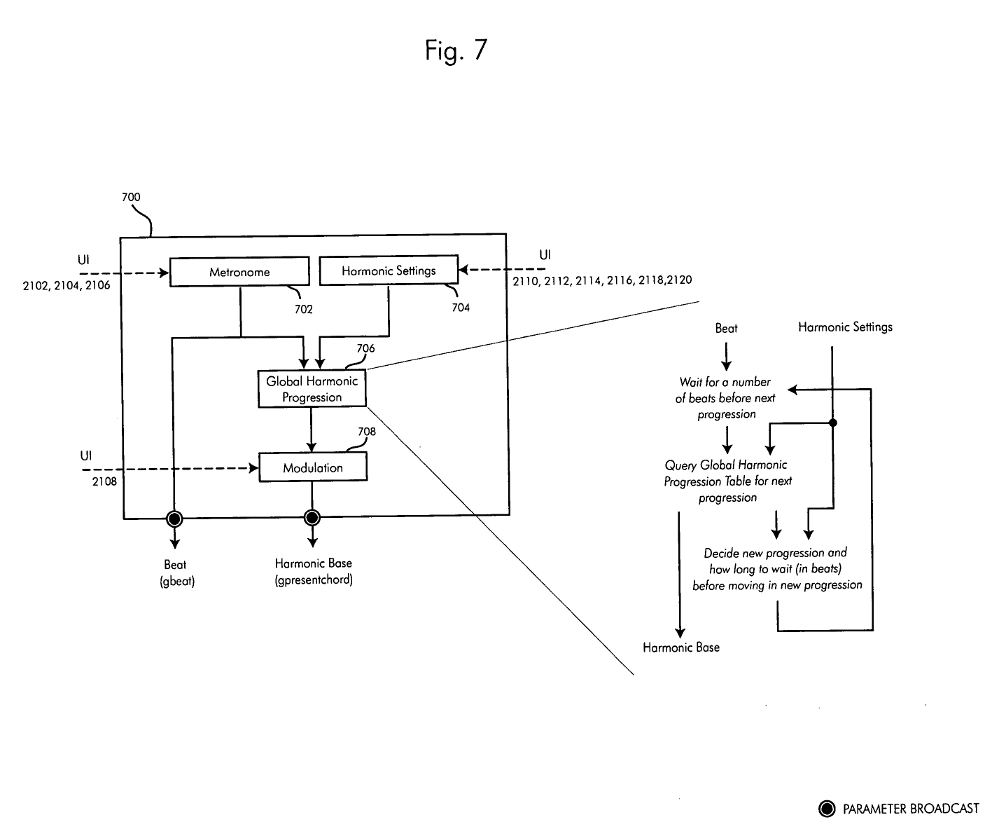

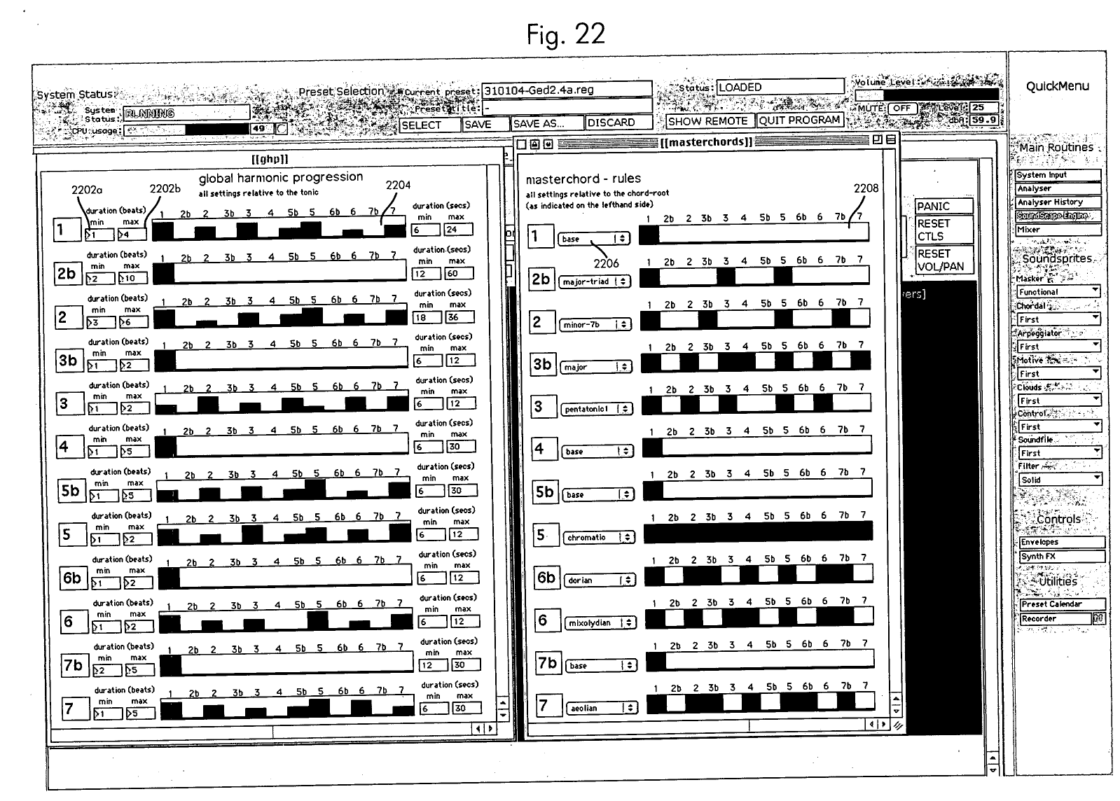

The outputs from the Analyser 500 are supplied to the Soundscape Base 108. The Soundscape Base 108 generates audio and MIDI outputs using the outputs from the Analyser 500, information received from the Intercom 122 and the Preset Manager 114, and internally generated information. The Soundscape Base 108 contains a Harmonic Brain 700, which, as shown in FIG. 7, contains multiple sub-modules that are supplied with information from the Preset Manager 114. The Harmonic Brain 700 contains a Metronome sub-module 702, a Harmonic Settings sub-module 704, a Global Harmonic Progression sub-module 706, and a Modulation sub-module 708, each of which receives user input information. The Metronome sub-module 702 supplies the global beat (gbeat) for the various modules in the Soundscape Base 108 and which is broadcast over the Intercom 122. The Harmonic Settings sub-module 704 receives the user input settings for the harmonic evolution of the system and the chord generation of the soundsprites. User settings include minimum and maximum duration settings for the system to remain in any possible pitchclass and weighted probability settings for the global harmonic progression of the system and the chord generation processes of the various soundsprites. The weighted probability user settings are set in tables containing multiple sliders corresponding to strength of probability for the corresponding pitchclass, as shown in FIG. 22. These settings and the duration user settings are stored by the Harmonic Settings sub-module 704 and are passed to the Global Harmonic Progression sub-module 706 and the soundsprite sub-modules 134, 152, 154, 156, 158 and 160. The Global Harmonic Progression sub-module 706 is also supplied with the outputs of the Metronome sub-module 702. The Global Harmonic Progression sub-module 706 waits for a number of beats before progressing to the next harmonic state. The number of beats is randomly selected between the minimum and the maximum number of beats supplied by the Harmonic Setting sub-module 704. Once the predetermined number of beats has been met, a global harmonic progression table is queried for the particular harmonic progression to use. After receiving this information from the Harmonic Setting sub-module 704, the harmonic progression is produced and supplied as a harmonic base to the Modulation sub-module 708. The Global Harmonic Progression sub-module 706 then decides how many beats to wait before starting a new progression. The Modulation sub-module 708 modulates the harmonic base dependent on user inputs. The modulation process in the Modulation sub-module 708 only becomes active if a new tonic center is supplied by the user and finds the best intermediate step and timing for moving the harmonic base to the supplied tonic. The Modulation sub-module 708 then outputs the modulated harmonic base. If user input is not supplied to the Modulation sub-module 708, the Harmonic Base output by the Global Harmonic Progression sub-module 706 passes through unaltered. The Modulation sub-module 708 supplies the Harmonic Base (gpresentchord) to the soundsprite sub-modules 134, 152, 154, 156, 158 and 160 and also broadcasts the harmonic base (gpresentchord) on the Intercom 122.

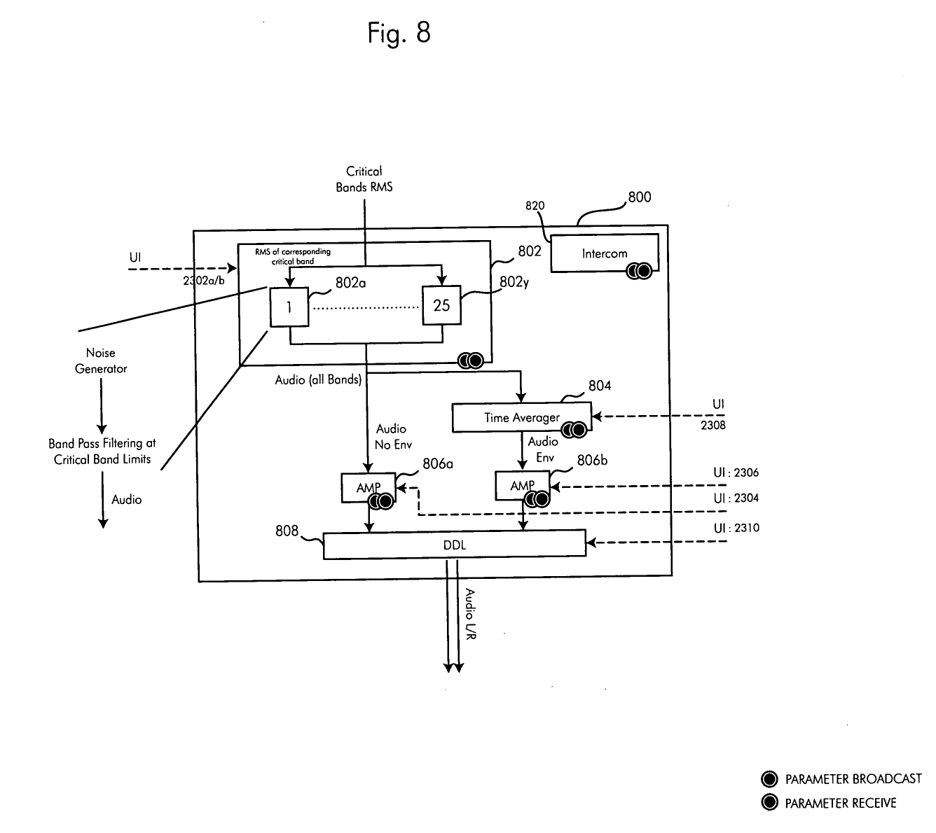

The Critical Band RMS from the Critical/Preset Band Analyser sub-module 506 of the Analyser 500 is supplied to the Functional Masker 800, as shown in FIG. 8. The critical bands RMS signal containing the 25 different RMS values for each of the critical bands shown in Table 1 is directed into an overall voice generator sub-module 802. The overall voice generator sub-module 802 contains a bank of voice generators 802a-802y, one per each critical band. Each voice generator creates white noise that is bandpass-filtered to the limits of its own critical band, using user inputs that determine the minimum and maximum band levels. The noise output of each voice is split into two signals: one which is smoothed by an amplitude envelope whose ramp time is variable by preset and one which is not. The smoothed filtered output uses a time averager sub-module 804 supplied with user inputs specifying the time over which the signal is averaged. The time-averaged signal, as well as the non-enveloped signal is then supplied to independent Amplifier sub-modules 806a and 806b which accept user inputs to determine the output levels of the two signals. The outputs of sub-modules 806a and 806b are then passed to a digital delay line (DDL) sub-module 808, which in turn is supplied with a user input that determines the length of the delay. The DDL sub-module 808 delays the signals before supplying them to the Mixer 114.

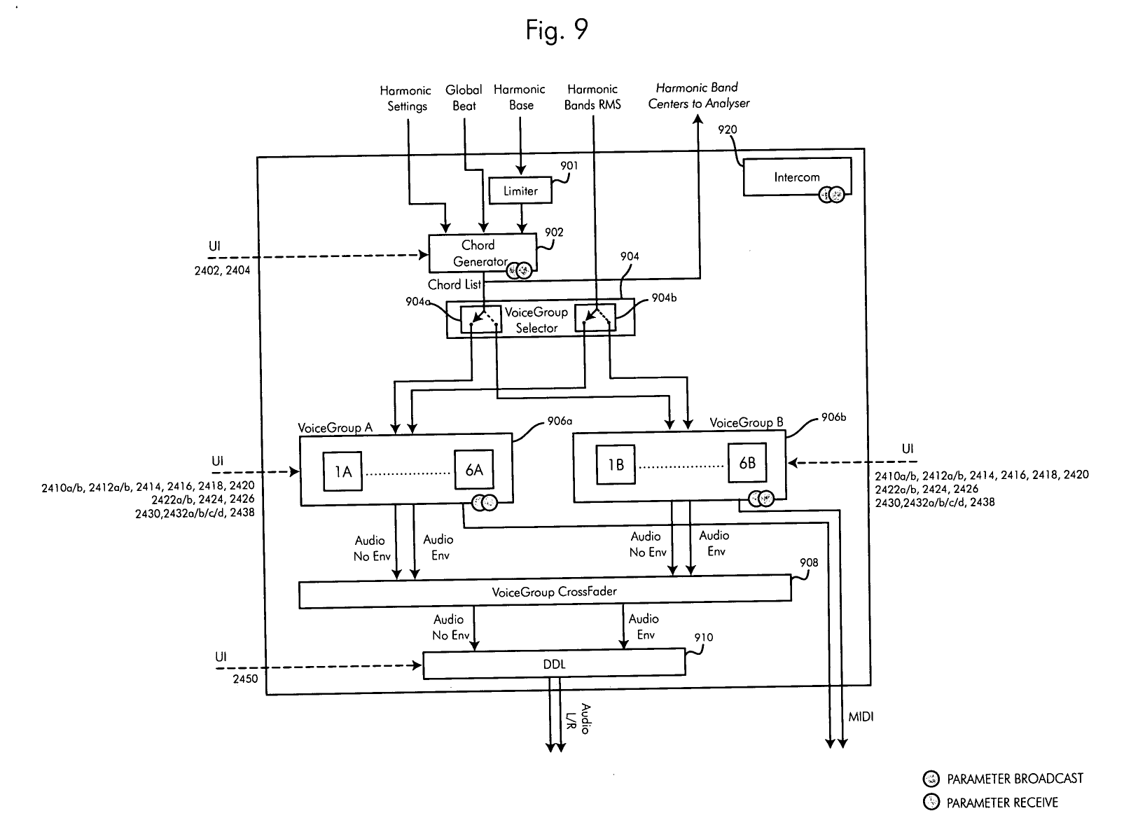

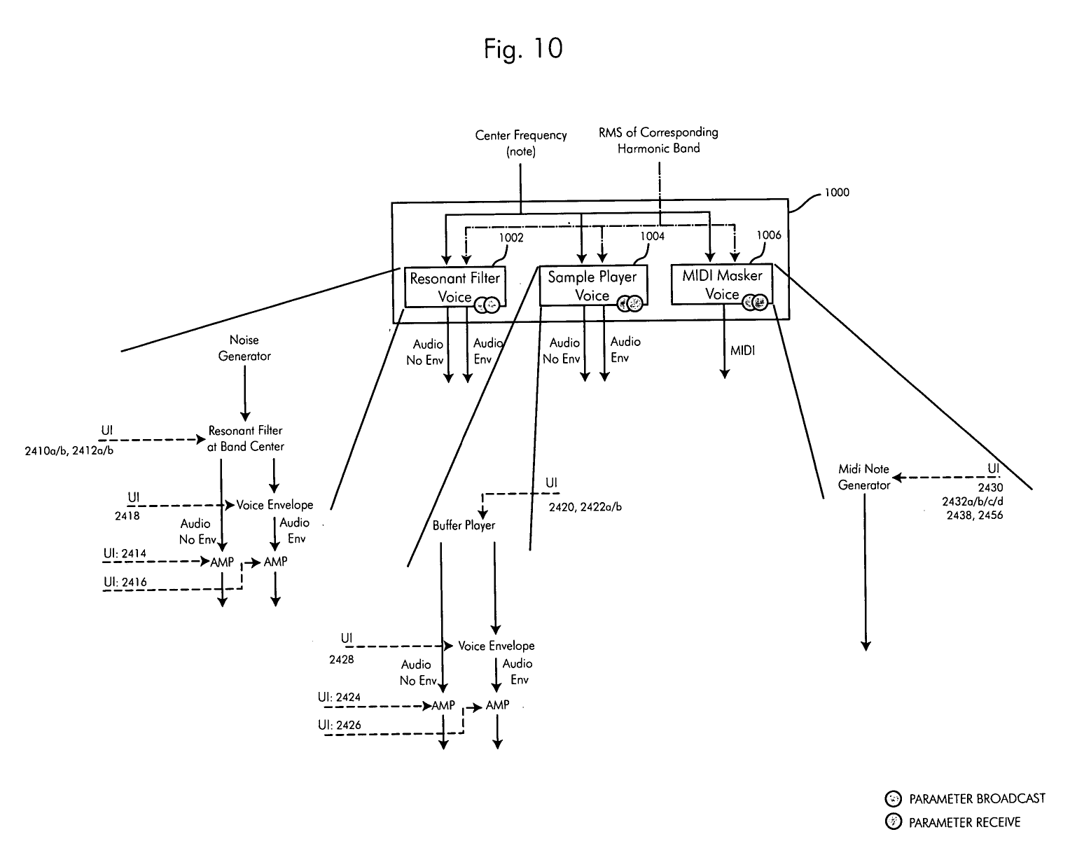

The Harmonic Masker 900, shown in FIGS. 9 and 10 is supplied with the RMS values of the harmonic bands from the Harmonic Band Analyser sub-module 508, as well as the global beat, the harmonic base and harmonic settings from the Harmonic Brain 170. The Harmonic Base received from the Harmonic Brain 170 is routed to a Limiter sub-module 901 and then to a Create Chord sub-module 902, which outputs a list of up to 6 pitchclasses, translated to corresponding frequencies. The Limiter sub-module 901 is a time gate that limits the rate of signals that are passed through. The Limiter sub-module 901 operates a gate, which closes when a new value passes though and reopens after 10 seconds. The number of pitchclasses and time after which the Limiter sub-module 901 reopens can vary as desired. The Chord sub-module 902 is supplied with user inputs including which Chord rule to use and the number of Notes to use. The pitchclasses are routed both to the Analyser 500 for analysis of the frequency spectrum in the harmonic bands, and to a Voice Group Selector sub-module 904.

The Voice Group Selector sub-module 904 routes the received frequencies together with the Harmonic Bands RMS values received from the Analyser 500 to either of two VoiceGroups A and B contained in Voice Group sub-modules 906a and 906b. The Voice Group Selector sub-module 904 contains switches 904a and 904b that alternate every time a new list of frequencies is received. Each VoiceGroup contains 6 Voicesets, a number of which (usually between 4 and 6) is activated. Each Voiceset corresponds to a note (frequency) produced in the Create Chord sub-module 902.

An enhanced view of one of the Voicesets 1000 is shown in FIG. 10. The Voicesets 1000 are supplied with the center frequencies (the particular notes) and the RMS of the corresponding harmonic band. The Voicesets 1000 contain three types of Voices supplied from a resonant filter voice sub-module 1002, a sample player voice sub-module 1004, and a MIDI masker voice sub-module 1006. The Voices build their output based on the center frequency received and at a level adjusted by the received RMS of the corresponding harmonic band.

The resonant filter voice sub-module 1002 is a filtered noise output. As in the Functional Masker 800, each voice generates two noise outputs: one with a smoothing envelope, one without. In the resonant filter voice sub-module 1002, a noise generator supplies noise to a resonant filter at the center of the band. One of the outputs of the resonant filter is provided to a voice envelope while the other is provided directly, without being subjected to the voice envelope, to an amplifier for adjusting their signal levels. The filter gain, steepness, minimum and maximum band level outputs, enveloped and non-enveloped signal levels, and enveloped signal time are controlled by the user.

The sample player voice sub-module 1004 provides a voice that is based on one or more recorded samples. In the sample player voice sub-module 1004, the center frequency and harmonic RMS are supplied to a buffer player that produces output sound by transposing the recorded sample to the supplied center frequency and regulating its output level according to the received harmonic RMS. The transposition of the recorded sample is effected by adjusting the duration of the recorded sample based on the ratio of the center for the harmonic band to the nominal frequency of the recorded sample. Similar to the noise generator of the resonant filter voice sub-module 1002, one of the outputs from the buffer player is then provided to a voice envelope while the other is provided directly, without being subjected to the voice envelope, to an amplifier for adjusting the signal levels. The sample file, minimum and maximum band level outputs, enveloped and non-enveloped signal levels, and enveloped signal time are controlled by the user.

The MIDI masker voice sub-module 1006 produces control signals for instructing the operation of the MIDI Synthesizer 112. The center frequency and harmonic RMS are supplied to a MIDI note generator, as are a user supplied MIDI voice threshold, an enveloped signal level and an enveloped signal time. The MIDI masker voice sub-module 1006 sends a MIDI instruction to activate a note in any of the harmonic bands when the harmonic RMS overcomes the MIDI voice threshold in that particular band. The MIDI masker voice sub-module 1006 also sends MIDI instructions to regulate the output level of the MIDI voice using the corresponding harmonic RMS. The MIDI instructions for the regulations of the MIDI voice output level are limited to, several, for example 10, instructions per second, in order to limit the number of MIDI instructions per second received by the MIDI synthesiser 110.

The outputs of the resonant filter voice sub-module 1002 and the sample player voice sub-module 1004, as shown in FIG. 9, are supplied to a VoiceGroup CrossFader sub-module 908. The VoiceGroup CrossFader sub-module 908 fades in and out the outputs of VoiceGroups A and B. Every time the switches 904a and 904b alternate for passing data to the other VoiceGroup, the VoiceGroup Crossfader sub-module 908 fades in the output of the new VoiceGroup and simultaneously fades out the output of the old VoiceGroup. The crossfading period is set to 10 secs, but any other duration can be used, provided that it is not longer that the time used in the Limiter sub-module 901. The enveloped signal and non-enveloped signal from the VoiceGroup CrossFader sub-module 908 is supplied to a DDL sub-module 910, which in turn is supplied with a user input that determines the length of the delay. The DDL sub-module 910 delays the signals before supplying them to the Mixer 114. The output from the MIDI masker voice sub-module 1006 is supplied directly to the MIDI Synthesiser 112. Thus, the output of the Harmonic Masker 900 is the mix of all the levels of each noise output of each voice employed.

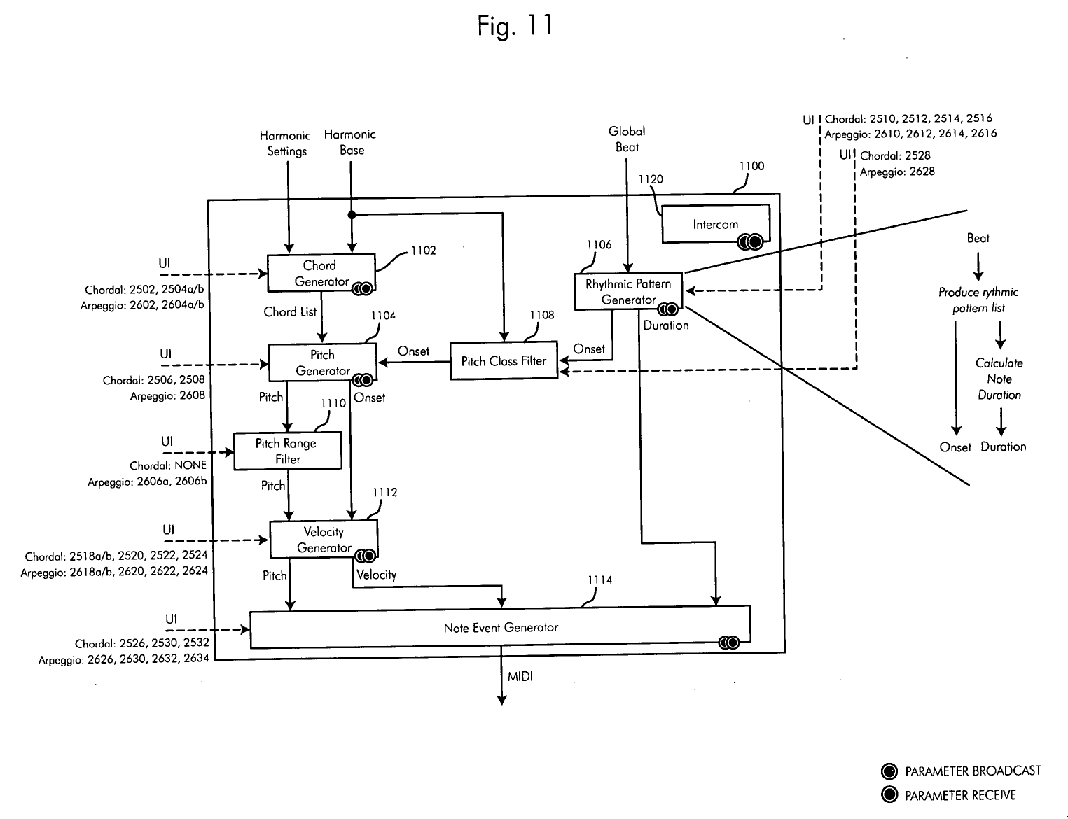

Turning now to FIGS. 11, 12, 13, 14 and 15, the generative soundsprites will be described. The generative soundsprites of one embodiment use either of two main generative methods: they create a set of possible pitches matching the currently active chord, or they create a number of pitches regardless of their relation to the current chord. The generative sound sprites employing the first method use the Harmonic Settings supplied by the Harmonic Brain 170 to select pitch classes corresponding to the Harmonic Base supplied by the Harmonic Brain 170. Of the soundsprites employing the second method, some have mechanisms in place to filter the pitches they generate to match to the current chord and others output the pitches they generate unfiltered.

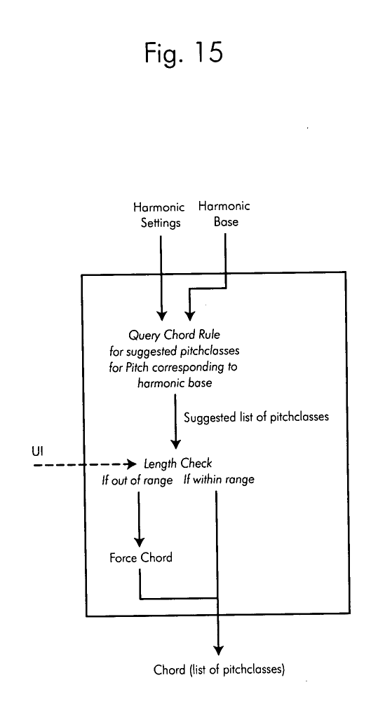

A view of one of the Arpeggiation and Chordal soundsprites 1100 is shown in FIG. 11. As shown in this figure, the harmonic base and harmonic settings from the Harmonic Brain 170 are supplied to a Create Generator sub-module 1102. The Chord Generator sub-module 1102 forms a chord list and provides the list to a Pitch Generator sub-module 1104. As shown in FIG. 15, the Chord Generator sub-module 1102 receives user inputs including which Chord rule to use (to determine which chord members should be selected) and the number of notes to use. The Chord Generator sub-module 1102 receives this information and determines a suggested list of possible pitchclasses for a pitch corresponding to the harmonic base. The lengths of the different possible chords are then checked to determine whether they are within the usable range. If the chord is within the usable range, the chord is supplied as is to the Pitch Generator sub-module 1104. If the chord is not within the usable range, i.e. if the number of suggested notes is higher than the maximum or lower than the minimum number of notes set by the user, then the chord is forced into the range and then again provided to the Pitch Generator sub-module 1104.

Meanwhile the global beat (gbeat) of the system is supplied to a Rhythmic Pattern Generator sub-module 1106. The Rhythmic Pattern Generator sub-module 1106 is supplied with user inputs so that a rhythmic pattern list is formed comprising 1 and 0 values, with one value generated for every beat. The onset for a note is produced whenever a non-zero value is encountered and the duration of the note is calculated by measuring the time between the current and the next non-zero values, or is used as supplied by the user settings. The onset of the note is transmitted to the Pitch Class filter sub-module 1108 and the duration of the note is passed to the Note Event Generator sub-module 1114.

The Pitch class filter sub-module 1108 receives the Harmonic Base from the Harmonic Brain 170 and user input to determine on which pitchclasses the current soundsprite is activated. If the Harmonic Base pitchclass corresponds to one of the selected pitchclasses, the Pitch class filter sub-module 1108 lets the Onset received by the Rhythmic pattern generator sub-module 1106 to pass through to the Pitch Generator 1104.

The Pitch Generator sub-module 1104 receives the chord list from the Chord Generator sub-module 1102 and the onset of the chord from the Pitch Class filter sub-module 1108 and provides the pitch and the onset as outputs. The Pitch Generator sub-module 1104 is particular for every different type of soundsprite employed.

The Pitch Generator sub-module 1104 of the Arpeggiation Soundsprite 154 stretches the Chord received by the Chord Generator 1102 to the whole midi-pitch spectrum then outputs the pitches selected and the corresponding note onsets. The pitches and note onsets are output, so that at every onset received by the Pitch Class Filter sub-module 1108, a new note of the same Arpeggiation chord is onset.

The Pitch Generator sub-module 1104 of the Chordal SoundSprite 152 transposes the Chord received by the Chord Generator 1102 to the octave band selected by user and then outputs the pitches selected and the corresponding note onsets. The pitches and note onsets are output, so that at every onset received by the Pitch Class Filter sub-module 1108 all the notes belonging to one chord are onset at the same time.

The Pitch Generator sub-module 1104 outputs the pitch to a Pitch Range Filter sub-module 1110, which filters the received pitches so that any pitch that is output is within the range set by the minimum and maximum pitch settings set by the user. The pitches that pass through the Pitch range Filter sub-module 1112 are then supplied to the Velocity Generator sub-module 1112.

The Velocity Generator sub-module 1112 derives the velocity of the note from the onset received from the Pitch Generator sub-module 1104, the pitch received from the Pitch range Filter sub-module 1112 and the settings set by the user and supplies the pitch and the velocity and to the Note Event Generator 1114.

The Note Event Generation sub-module 1114 receives the pitch, the velocity, the duration of the note and the supplied user settings and creates note event instructions, which are sent to the MIDI synthesizer 112.

The Intercom sub-module 1120 is operating within the soundsprite 1100 to route any of the available parameters on the Intercom receive channels to any of the generative parameters of the soundsprite, otherwise set by user settings. The generated parameters within the soundsprite 1100 can then in turn be transmitted over any of the Intercom broadcast channels dedicated to this particular soundsprite.

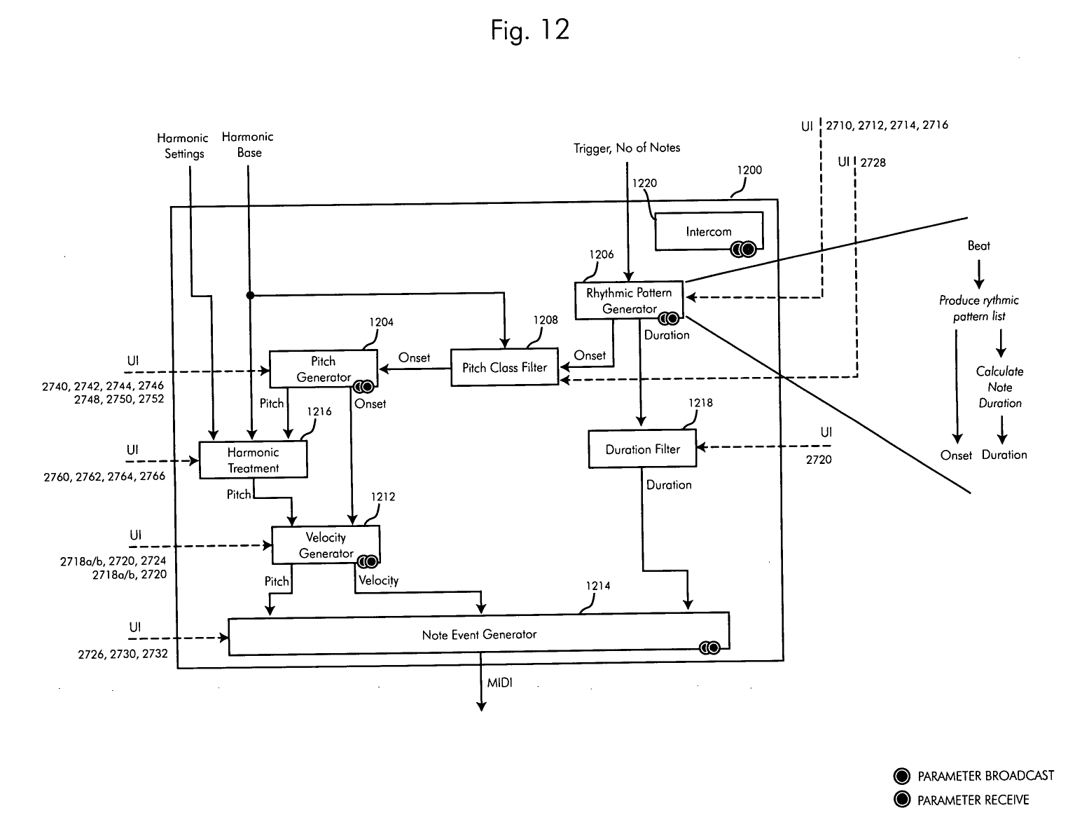

The Motive soundsprite 158 is similar to the motive voice in the applications incorporated by reference above. Thus, the Motive soundsprite 158 is triggered by prominent sound events in the acoustical environment. An embodiment of the Motive soundsprites 1200 will now be described with reference to FIG. 12. As shown in this figure, a Rhythmic Pattern Generator sub-module 1206 receives a trigger signal. The trigger signal is an integer usually sent by the appropriate local Intercom channel and constitutes the main activation mechanism in this embodiment of the Motive soundsprite 156. The integer received is also the number of notes that will be played by the Motive Soundsprite 156. The Rhythmic Pattern Generator sub-module 1206 has similar function to the Rhythmic Pattern Generator sub-module 1106 described above, but in this case it outputs a number of onsets, and corresponding duration signals, equal to the number of notes received, as a trigger. Also, during the process of pattern generation, the Rhythmic Pattern Generator sub-module 1206 closes its input gate so no further trigger signals can be received until the current sequence is terminated. The Rhythmic Pattern Generator sub-module 1206 outputs are the duration to a Duration Filter sub-module 1218 and Onset to the Pitch class Filter sub-module 1208. The Duration Filter sub-module 1218 controls the received duration so that it does not exceed a user set value. Also, it can accept user settings to control the duration, thus overriding the Duration received from the Rhythmic Pattern Generator sub-module 1206. The Duration Filter sub-module 1218 then outputs the Duration to the Note Event Generator 1214.

The Pitch Class filter sub-module 1208 performs the same function as the Pitch Class filter sub-module 1108 described above and outputs the onset to the Pitch Generator 1204.

The Pitch Generator sub-module 1204 receives the onset of a note from the Pitch Class filter sub-module 1208 and provides the pitch and the onset as outputs, following user set parameters that regulate the selection of pitches. The user settings are applied as interval probability weightings that describe the probability of a certain pitch to be selected in relation to its tonal distance from the last pitch selected. The user settings applied also include setting of centre pitch and spread, maximum number of small intervals, maximum number of big intervals, maximum number of intervals in one direction and maximum sum of a row in one direction. Within the Pitch Generator sub-module 1204, intervals bigger than or equal to a fifth are considered big intervals and intervals smaller than a fifth are considered small intervals.

The Pitch Generator sub-module 1204 outputs the note pitch to a Harmonic Treatment sub-module 1216 which also receives the Harmonic Base and Harmonic Settings and user settings. The user settings define any of three states of harmonic correction, namely ‘no correction’, ‘harmonic correction’ and ‘snap to chord’. In the case of ‘harmonic correction’ or ‘snap to chord’ user settings also define the harmonic settings to be used and in the case of ‘snap to chord’ they additionally define the minimum and maximum number of notes to snap to in a chord.

When the Harmonic Treatment sub-module 1216 is set to ‘snap to chord’, a chord is created on each new Harmonic Base received from the Harmonic Brain 170, which is used as a grid for adjusting the pitchclasses. For example, in case a ‘major triad’ is selected as the current chord, each pitchclass running through the Harmonic Treatment sub-module 1216 will snap to this chord by being aligned its closest pitchclass contained in the chord.

When the Harmonic Treatment sub-module 1216 is set to ‘harmonic correction’ it is determined how pitchclasses should be altered according to the current harmonic settings. For this setting, the interval probability weightings settings are treated as likeliness percentage values for a specific pitch to pass through. For example, in case the value at table address ‘0’ is ‘100’, pitchclass ‘0’ (midi-pitches 12, 24 etc.) will always pass unaltered. In case the value is on ‘0’, pitchclass ‘0’ will never pass. In case it is ‘50’, pitchclass ‘0’ will pass half of the times on average. In case the currently suggested pitch is higher than the last note and didn't pass through the first time, its pitch is increased by 1 and the new pitch is tried recursively for a maximum of 12 times until it is abandoned.

The Velocity Generator sub-module 1212 receives the Pitch from the Harmonic Treatment sub-module 1216, the Onset from the Pitch Generator 1204 and the settings supplied by user settings and derives the velocity of the note which is output to the Note Event Generator 1214 together with the Pitch of the note.

The Note Event Generator sub-module 1214 receives the pitch, the velocity, the duration of the note and the supplied user settings and creates note event instructions, which are sent to the MIDI synthesizer 112.

The Intercom sub-module 1220 operates within the soundsprite 1200 in a similar fashion described above for the soundsprites 1100.

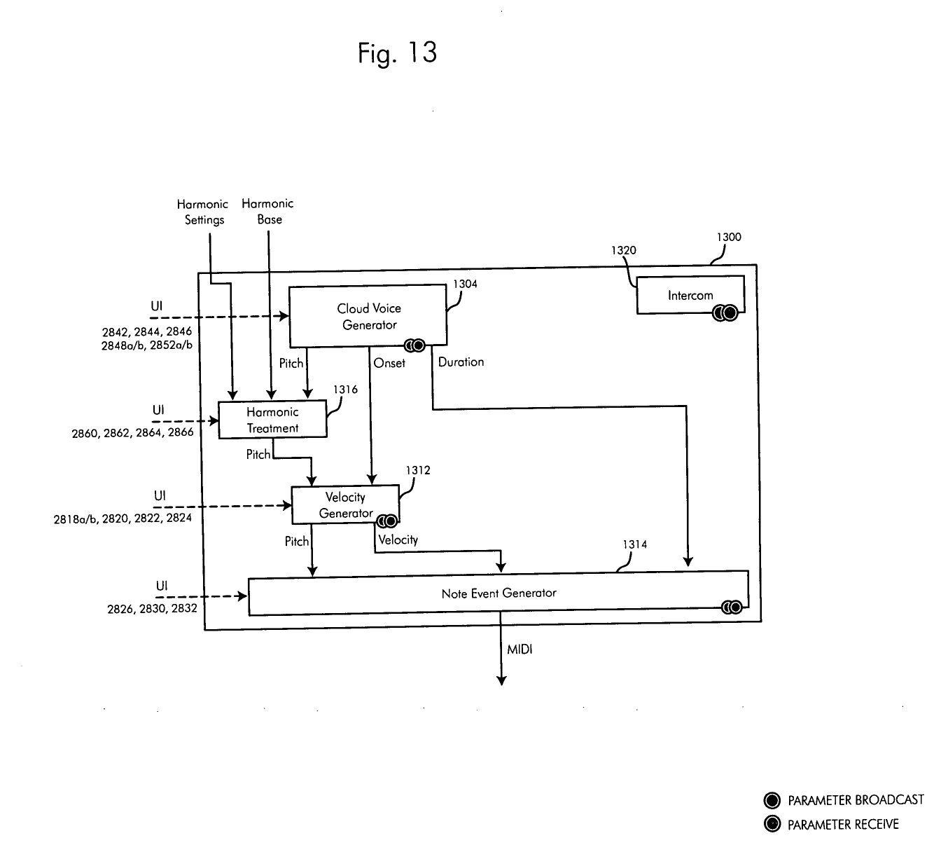

Turning now to FIG. 13, the Clouds soundsprite 160 will be described.

The Clouds soundsprite 160 creates note events independent of the global beat of the system (gbeat) and the number of beats per minute (bpm) settings from the Harmonic Brain 170.

The Cloud Voice Generator sub-module 1304 accepts user settings and uses an internal mechanism to generate Pitch, Onset and Duration. The user input interface (also called Graphical User Interface or GUI) for the Cloud Voice Generator sub-module 1304 includes a multi-slider object on which different shapes may be drawn which are then interpreted as the density of events between the minimum and maximum time between note events (also called attacks). User settings also define the minimum and maximum times between note events and pitch related information, including center pitch, deviation and minimum and maximum pitch. The generated pitches are passed to a Harmonic Treatment sub-module 1316, which functions as described above for the Harmonic Treatment sub-module 1216 and outputs pitch values to a Velocity Generator sub-module 1312. The Velocity Generator sub-module 1312, the Note Event Generator sub-module 1314 and the Intercom sub-module 1320 also have the same functionality as described earlier.

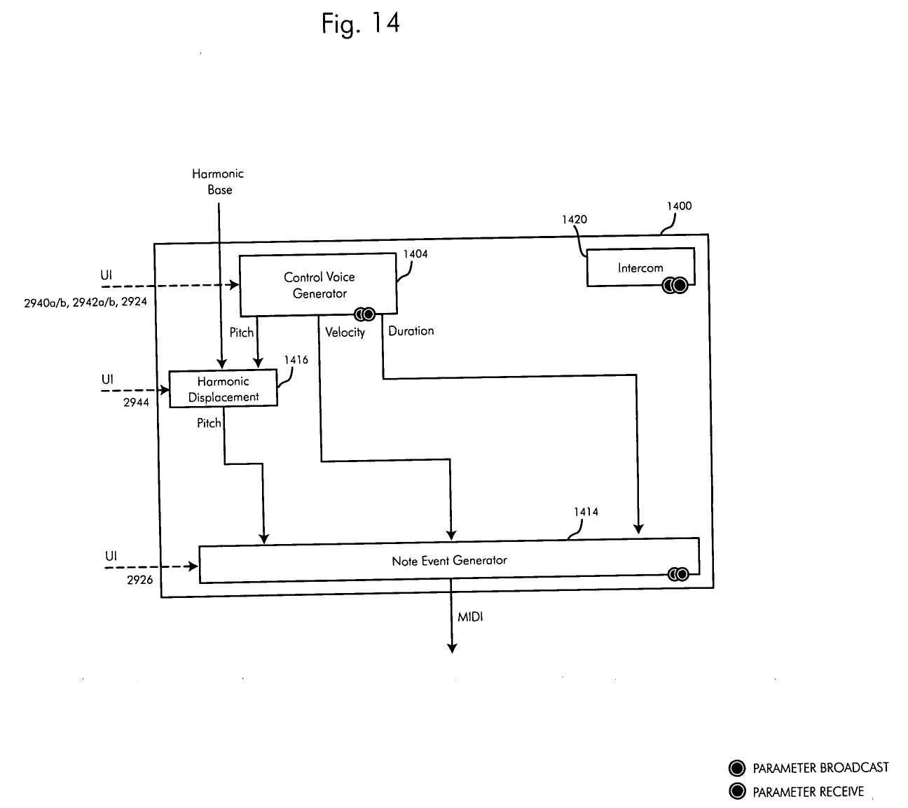

Turning now to FIG. 14, the Control soundsprite 158 will be described.

The Control soundsprite 158 is used to create textures rather than pitches. Data is transmitted to the Control soundsprite 1400 on the Intercom 1416 and from the Harmonic Brain 170.

The Control Voice Generator 1404 creates data for notes of random duration within the range specified by the user with minimum and maximum duration of note events. In between the created notes are pauses of minimum or maximum duration according to user settings. The Control Voice Generator 1404 outputs a pitch to the Harmonic Displacement sub-module 1416, which uses the Harmonic Base provided by the Harmonic Brain 170 and offsets/transposes this by the amount set by the user settings. The Note Event Generator sub-module 1414 and the Intercom sub-module 1420 operate in the same fashion as described above.

The Soundfile soundsprite 144 plays sound files in AIF, WAV or MP3 format, for example, in controlled loops and thus can be directly applied to the Mixer 112 for application to the speakers or other device that transforms the signals into acoustic energy. The sound files may also be stored and/or transmitted in some other comparable format set by the user or adjusted as desired for the particular module or device into which the signals from the Soundfile soundsprite 144 is input. The output of the Soundfile soundsprite 144 can be conditioned using the Analyser 104 and other data received over the Intercom 122.

The solid filter 136 sends audio signals routed to it through an 8-band resonant filter bank. Of course, the number of filters may be altered as desired. The frequencies of the filter bands can be set by either choosing one or more particular pitches from a list of available pitches via user selection on the display or by receiving one or more external pitches through the Intercom 122.

The Intercom 122 will now be described in more detail with reference to FIGS. 3 and 38-45. As described before, most of the modules use the Intercom 122. The Intercom 122 essentially permits the sound screening system 100 to have a decentralized model of intelligence so that many of the modules can be locally tuned to be responsive to specific parameters of the sensed input, if required. The Intercom 122 also allows the sharing of parameters or data streams between any two modules of the sound screening system 100. This permits the sound designer to design sound presets with rich reaction patterns of soundsprites to external input and of one soundsprite to the other (chain reactions). The Intercom 122 operates using “send” objects that broadcast information in available intercom channels and “receive” objects that can receive this information and route the information to local control parameters.

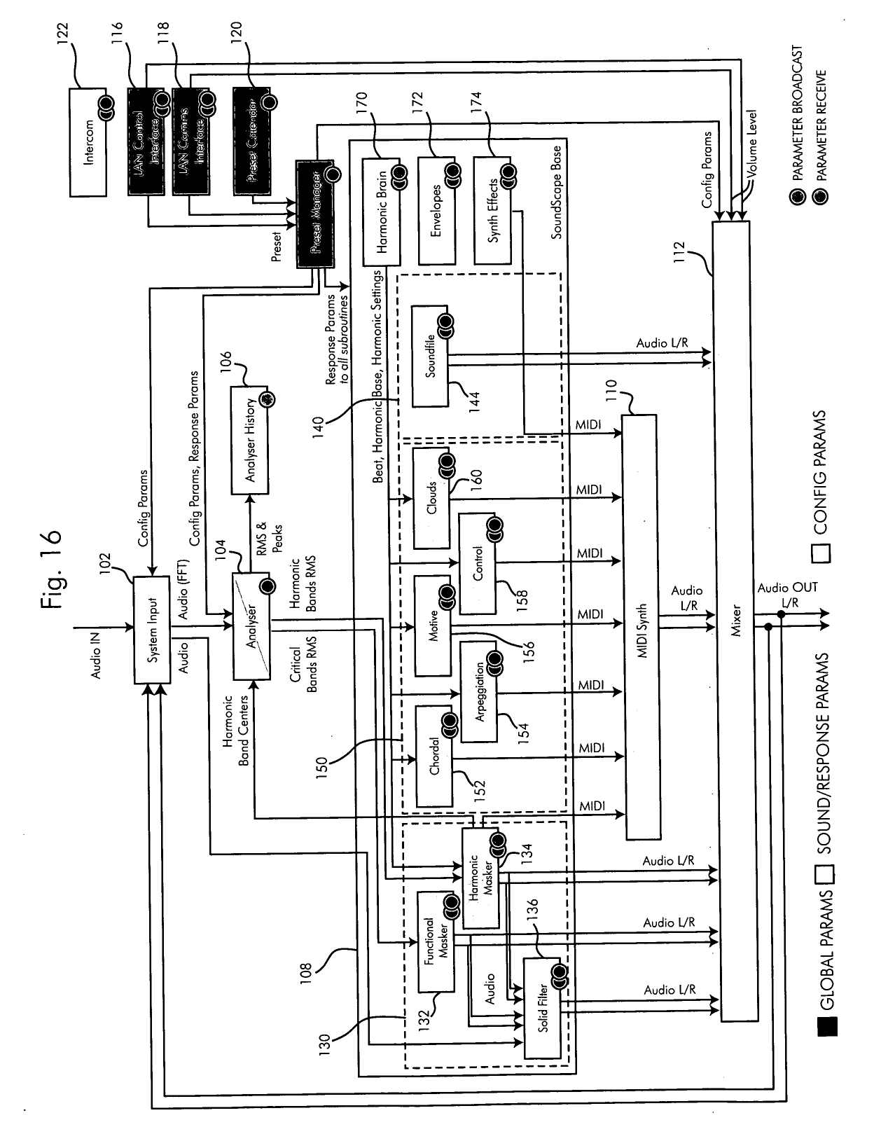

All user parameters, which are set to define the overall response of the algorithm, are stored in presets. These presets can be recalled as required. The loading/saving of parameters from/to preset files is handled by the Preset Manager 114. FIG. 16 is a representation of the system components/subroutines per parameter type. User parameters are generally of three types: global, configuration and sound/response parameters. The global parameters may be used by the modules throughout the sound screening system 100, the sound/response parameters may be used by the modules in the Soundscape Base 108 as well as the Analyser 104 and the MIDI synthesizer 110, and the configuration parameters may be used by the remainder of the modules as well as the Analyser 104.

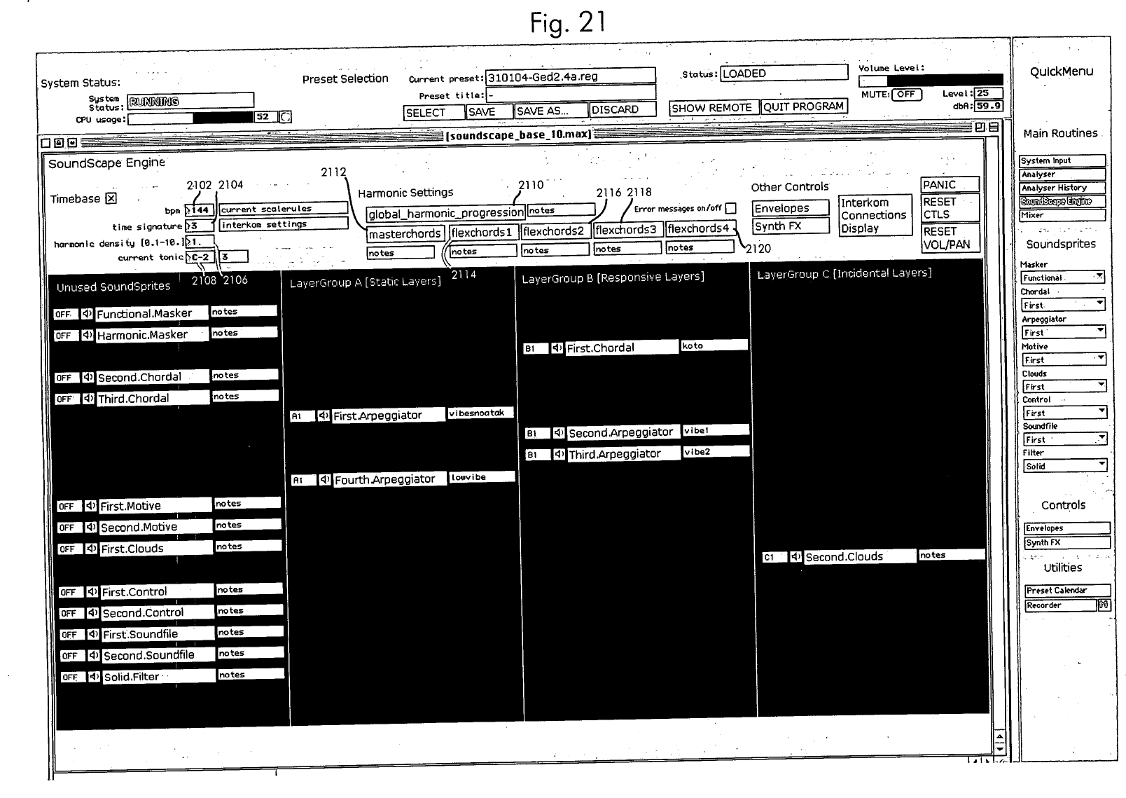

In a specific example of parameter setup and sharing, shown in FIGS. 21 and 38-45, soundsprites can be set to belong in one of multiple layers. In the embodiment shown, 7 layers have been chosen. These layers are grouped in 3 Layergroup as follows: Layergroup 1, consisting of Layers 1A, 1B and 1C; Layergroup 2, consisting of Layers 2A and 2B; and Layergroup 3, consisting of Layers 3A and 3B. The intercom receive channels are context sensitive depending on the position of a soundsprite in any of these layers. In a soundsprite belonging to Layer B1 the following intercom parameters are available:

TABLE 2

| Available Parameter | Type | Broadcast by |
|---|---|---|
| ‘Layergroup_B_1’ to | Parameters available only within | Soundsprites on |
| ‘Layergroup_B_5’ | the Layergroup B | Layergroup B |
| ‘Layer_B1_1’ to | Parameters available only within | Soundsprites on |
| ‘Layer_B1_5’ | Layer B1 | Layer B1 |
| ‘RMS_A’, ‘RMS_B’, | RMS values of user set Frequency | ANALYSER |
| ‘RMS_C’, ‘RMS_D’, | Bands | |
| ‘PEAKS_A’, “PEAKS_B’, | PEAK events within user set | ANALYSER |
| ‘PEAKS_C’, ‘PEAKS_D’ | frequency bands | |
| ‘RMS_A_10 min’, | RMS values of user set Frequency | ANALYSER |
| ‘RMS_A_60 min’, | Band A averaged over time spans of | HISTORY |
| ‘RMS_A_24 hour’ | 10 min, 60 min, 24 hours | |
| ‘PEAKS_A_10 min’, | PEAK counts within user set | ANALYSER |
| ‘PEAKS A_60 min’, | Frequency Band A over longer time | HISTORY |
| ‘PEAKS_A_24 hour’ | spans to 10 min, 60 min, 24 hours | |
| ‘gpresentchord’ | current harmonic base | Harmonic Brain |
| ‘secs’ | Beat every second | System Clock |
| ‘mins’ | Beat every minute | System Clock |
| ‘hours’ | Beat every hour | System Clock |
| ‘global_1’ to ‘global_10’ | Parameters available globally in the | Soundsprites on |
| | system | any Layer |
| ‘env_1’ to ‘env_16’ | User set Envelopes | Envelop utility |

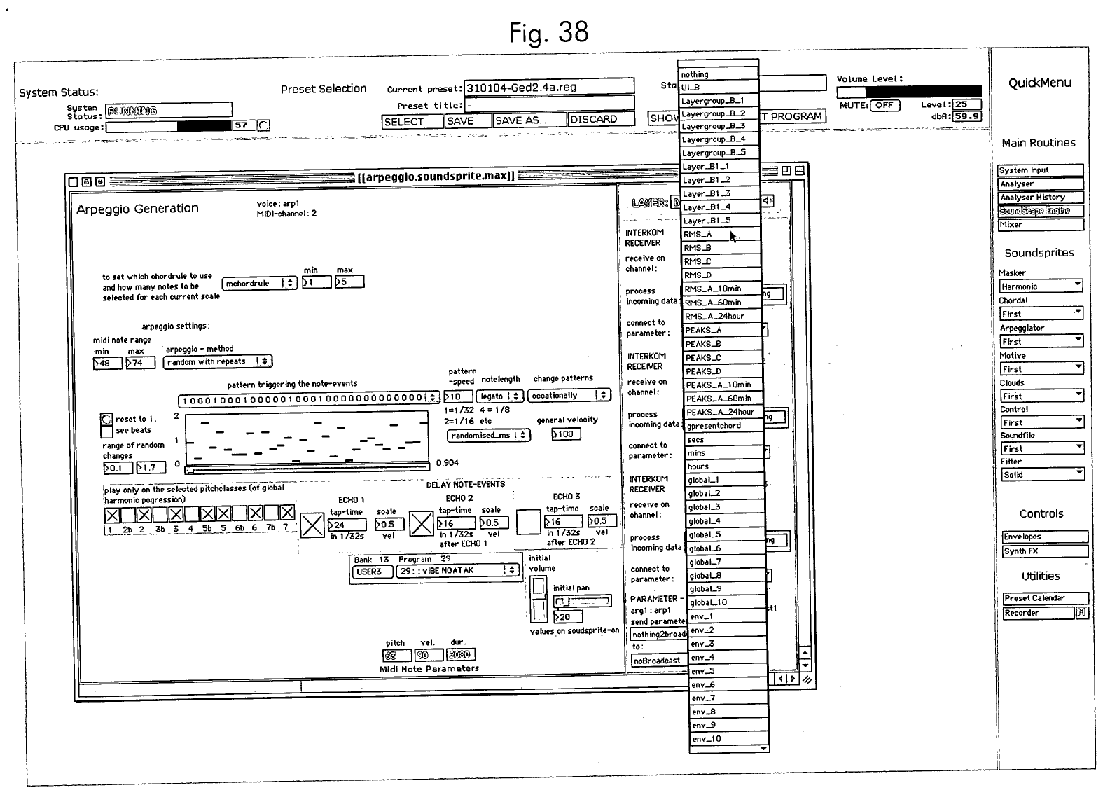

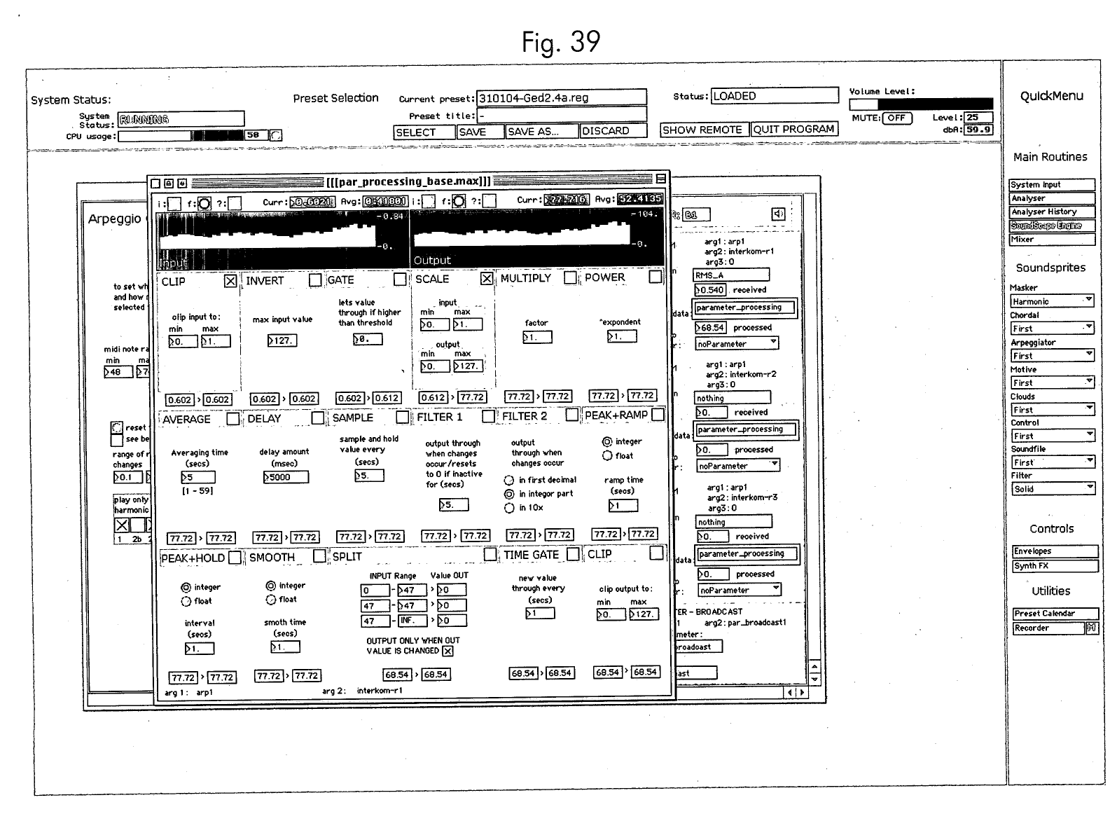

As shown in FIGS. 38-45, the parameter broadcast and pick-up are set via drop-down menus in the GUI. The number of channels and groups, as well as the arrangement of the groups, used by the Intercom 122 are arbitrary and may depend on the processing ability, for example, of the overall sound screening system 100. To allow for the conditioning of the received parameter to suit the parameter that the user might want to have dynamically adjusted a parameter processing routine is employed as shown in FIG. 39. One parameter processing routine is available for every intercom receive menu.

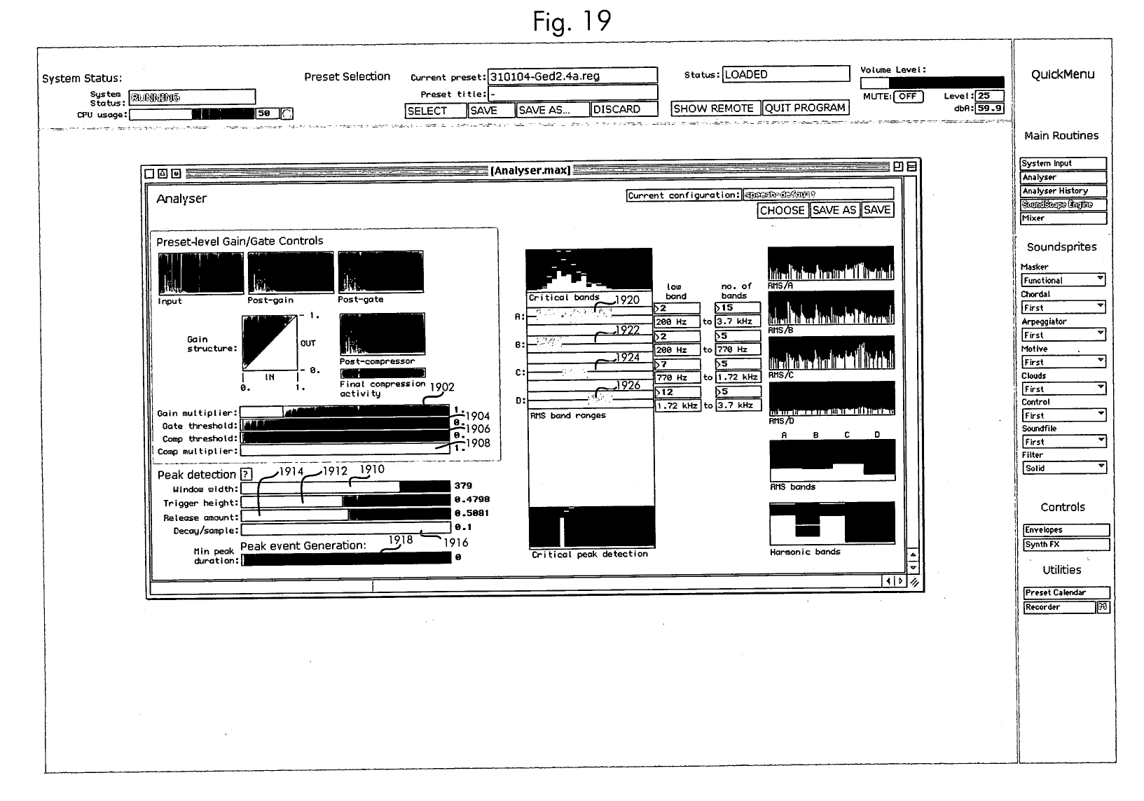

In one example, shown in FIGS. 19 and 38-44, the use of the Intercom 122 for setting up an input-to-soundsprite and a soundsprite-to-soundsprite relation is described. In this example, it is desired to have the Velocity of an arpeggio soundsprite belonging in layer B 1 dynamically adjusted by the RMS value of the spectrum of the sensed input between 200 Hz to 3.7 KHz and to broadcast the volume of the arpeggio within the system for use in a soundsprite in layer C2. The Intercom channel of the Arpeggio soundsprite shown in FIG. 38 is set so that the Arpeggio soundsprite belongs to layer B1.

The procedure starts by defining a particular frequency band in the Analyser 104. As shown in the uppermost window on the right hand side of the Analyser window in FIG. 19, the boundaries of Band A in the Analyser 104 are set to be between 200 Hz and 3.7 KHz. As illustrated, a graph of RMS_A is present in the topmost section to the right of the selector. The graph of RMS_A shows the history of the value.

Next, RMS_A is received and connected to General Velocity. To accomplish this, the user goes to the Arpeggio Generation screen in FIG. 38, clicks on one of the intercom receive pull down menus on the right hand side, and selects RMS_A from the pull down menu. The various parameters available as an input are shown in FIG. 38. The parameter processing window (shown as ‘par processing base.max’) appears as shown in FIG. 39. As can be seen in the input graph marked ‘input’ in the top left hand side of the parameter processing window, RMS_A is a floating quantity having values between 0 and 1. The input value can be appropriately processed using the various available processes provided. In this case the input value is ‘clipped’ within a range of a minimum of 0 and a maximum of 1 and is then scaled so that the output parameter is an integer with a value between 1 and 127 as shown in the sections marked ‘CLIP’ and ‘SCALE’ which have been activated. The current value and the recent history of the Output value resulting from the applied parameter processing is shown in the Graph marked ‘Output’ in the top right corner of the parameter processing window.

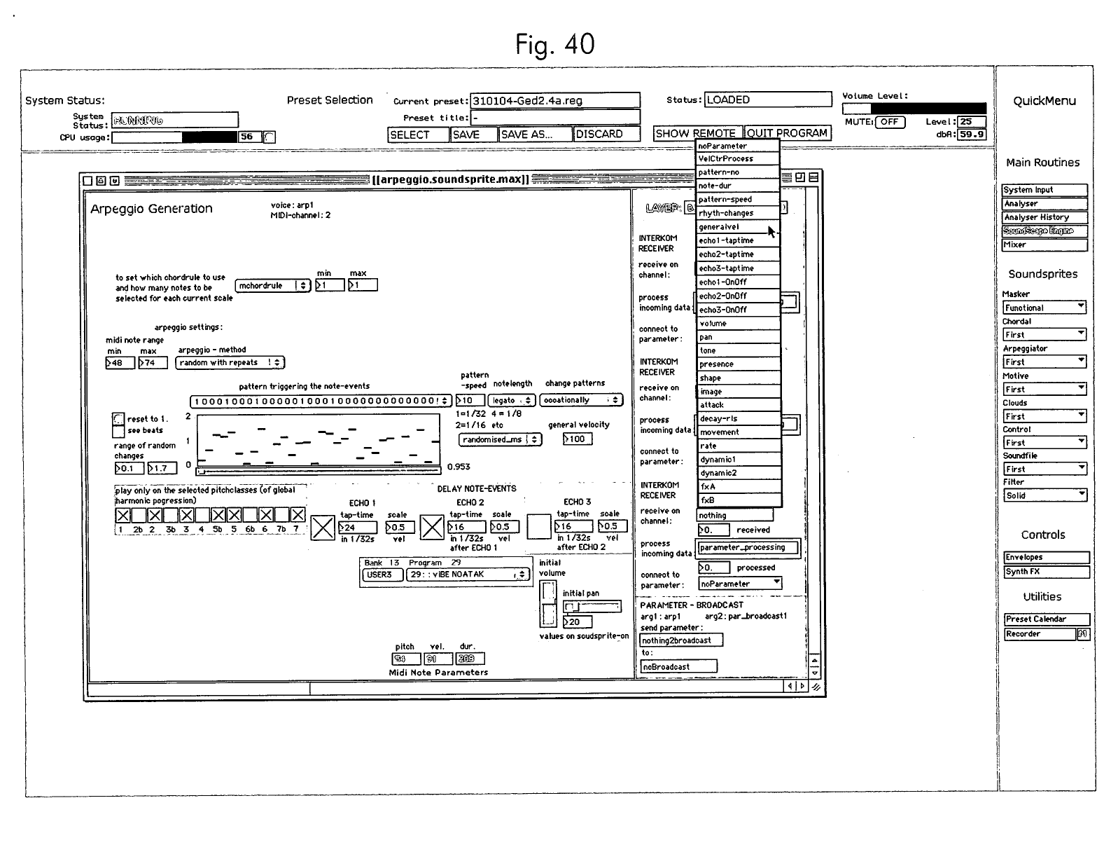

To connect the parameter received on the receive Channel of the Intercom Receiver (RMS_A) to the General Velocity parameter of the Arpeggio Soundsprite 154, the user next chooses ‘generalvel’ in the ‘connect to parameter’ drop down menu in the same top section, below the intercom receive selector. The various parameters available for linking are shown in FIG. 40.

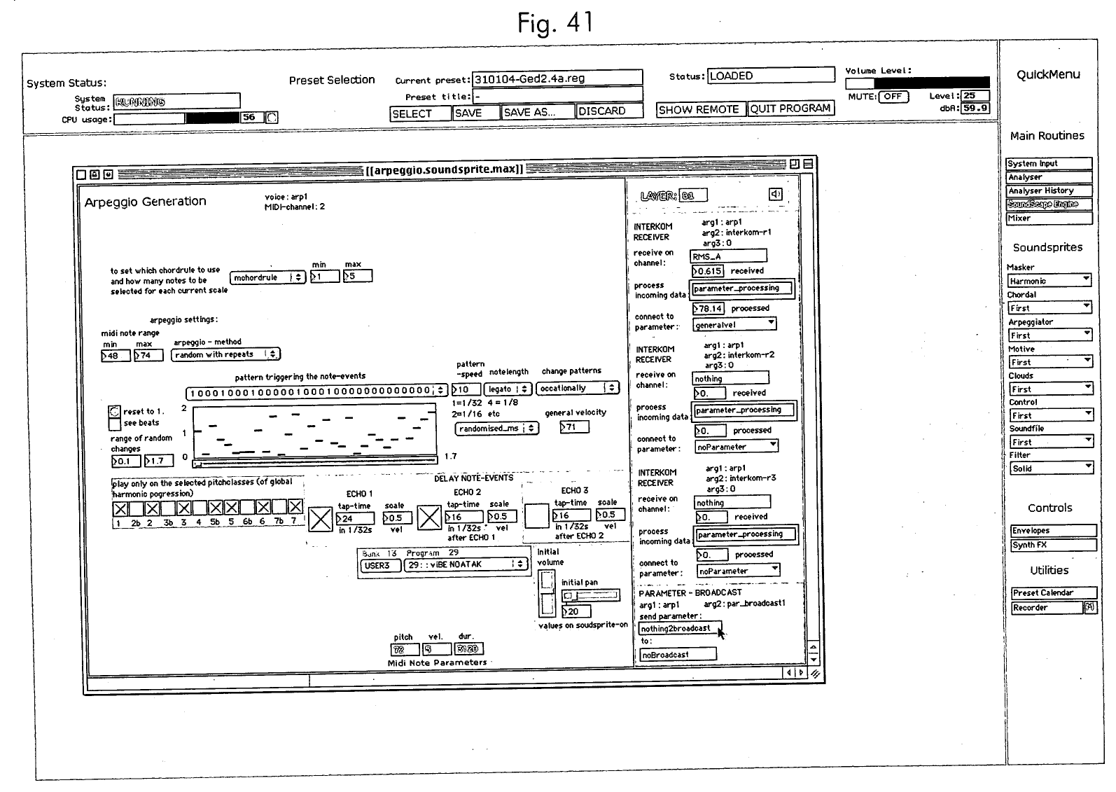

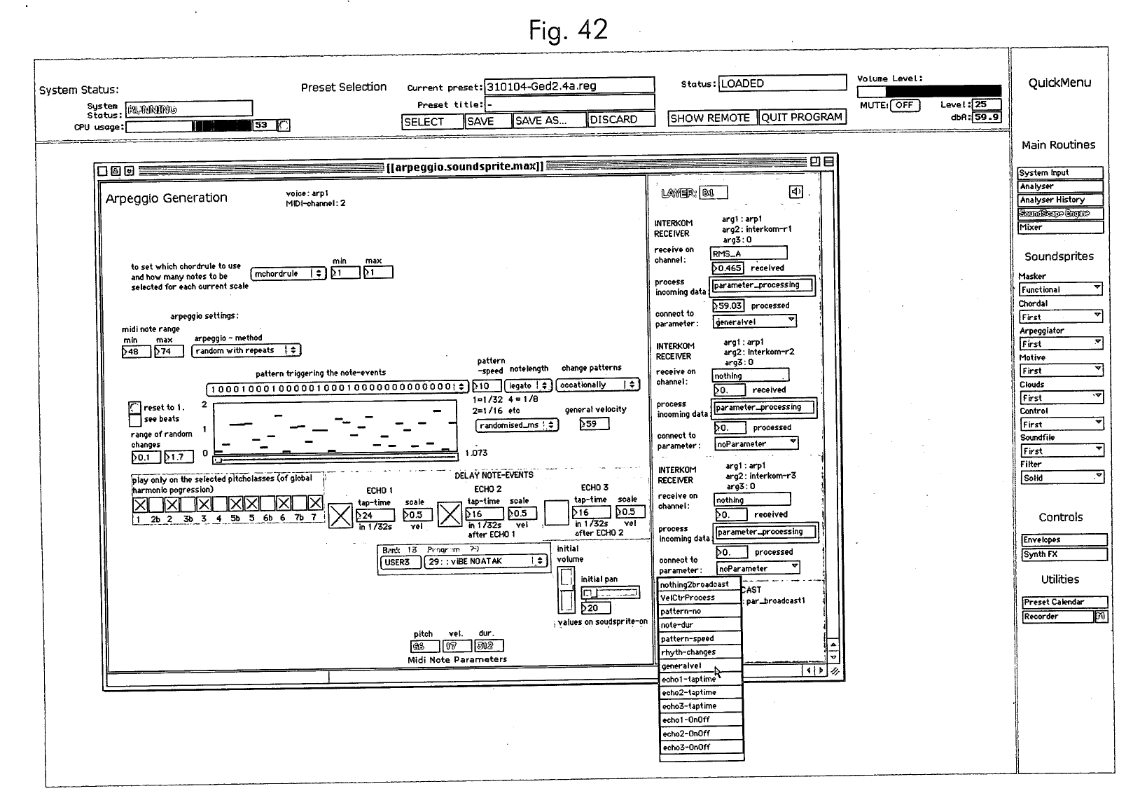

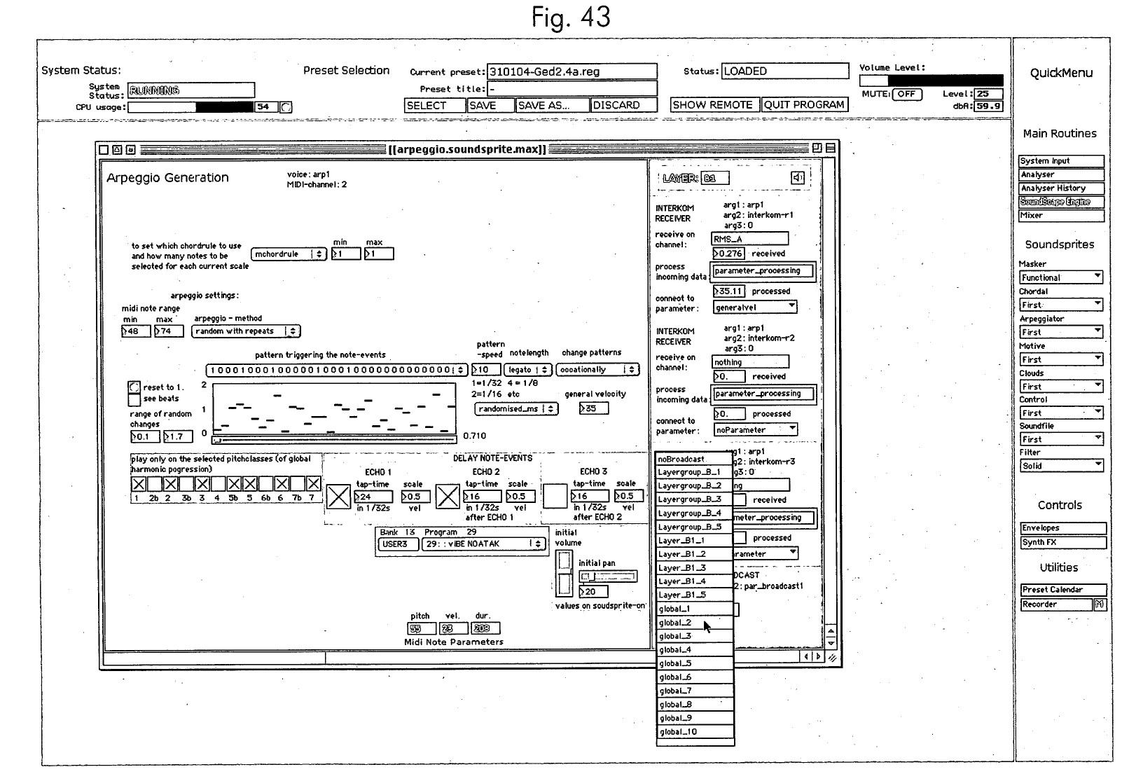

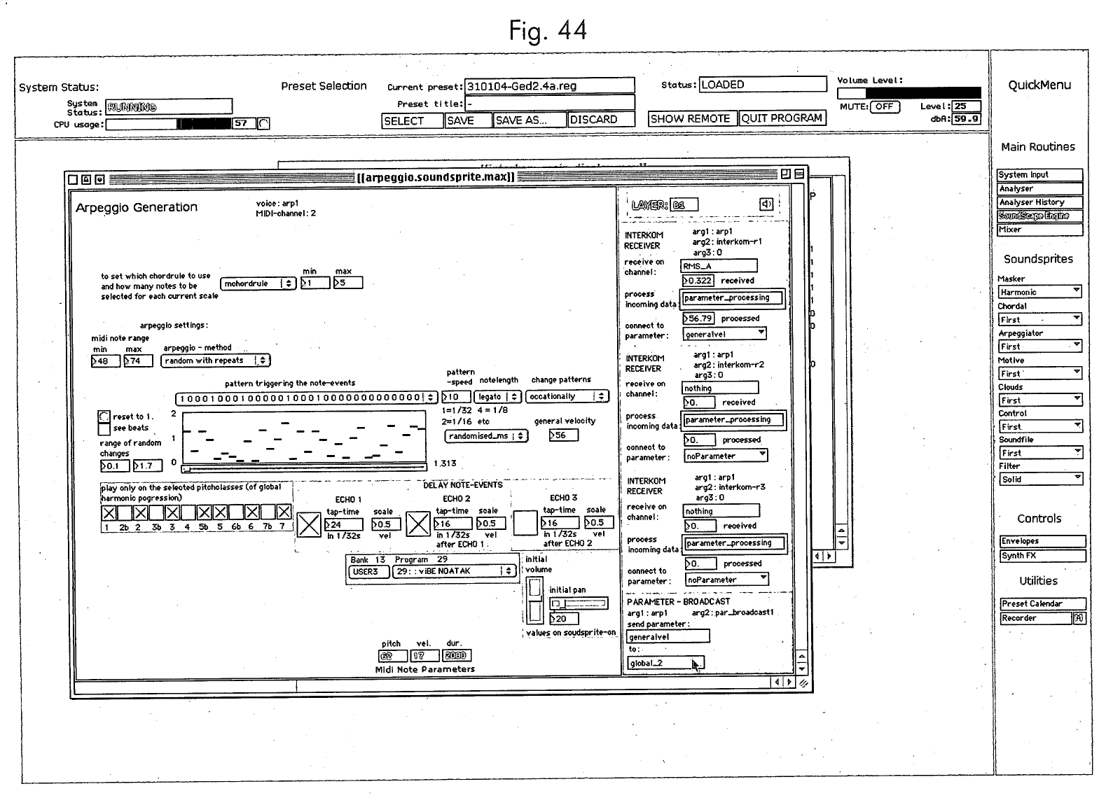

The linkage between RMS_A and Volume is more clearly shown in FIG. 41 as the top box on the right hand side also called interkom-r1. FIGS. 41 to 44 illustrate the broadcasting of the dynamically adjusted volume along one of the intercom Broadcast channels. FIG. 41 shows the “PARAMETER BROADCAST” section in the bottom right of the soundsprite GUI before a particular channel is selected. The “nothing to broadcast” tab in the “PARAMETER BROADCAST” section is clicked on and “generalvel’ is selected as shown in FIG. 42. In FIG. 43, the ‘to’ tab underneath is selected, and one of the parameters, e.g. global_2, is selected, if it is available. FIG. 44 illustrates the intercom settings that have been set for the Intercom receive and Parameter Broadcast channels.

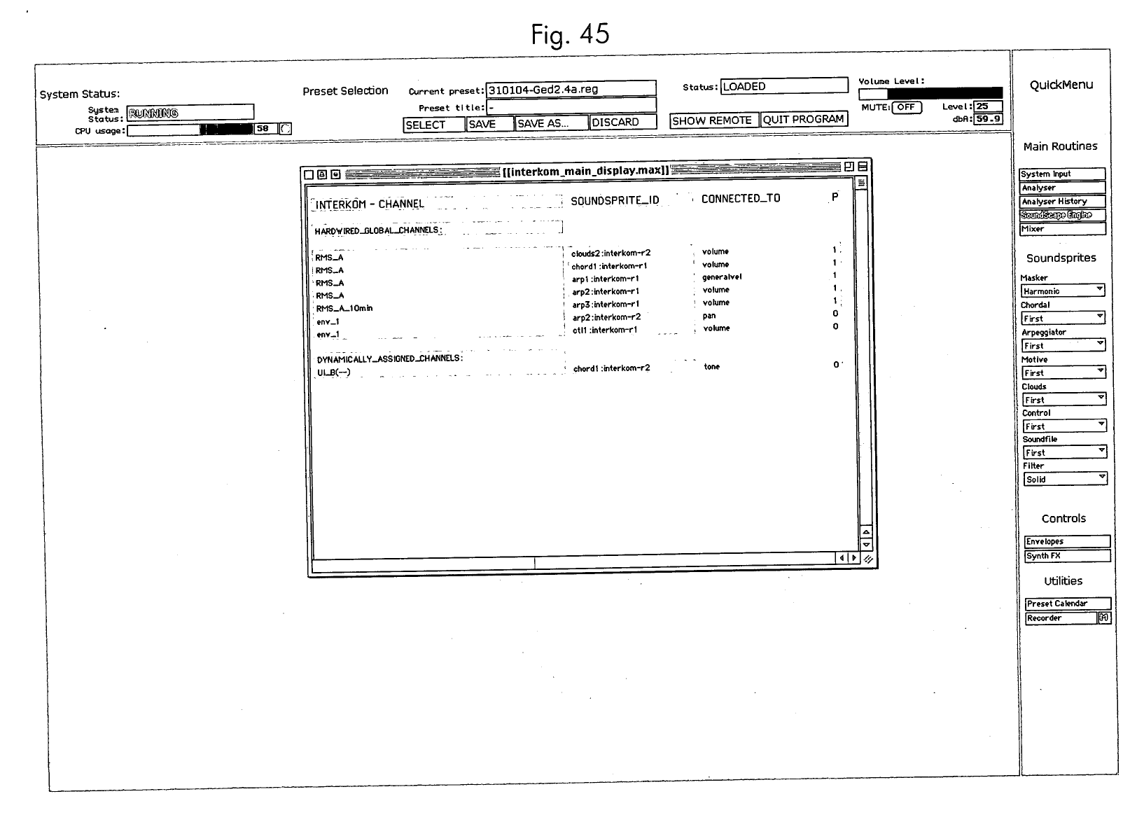

The connections established through the Intercom between the available parameters of the sound screening system 100 is shown in FIG. 45, which shows a pop-up window updated with all the Intercom Connections information.

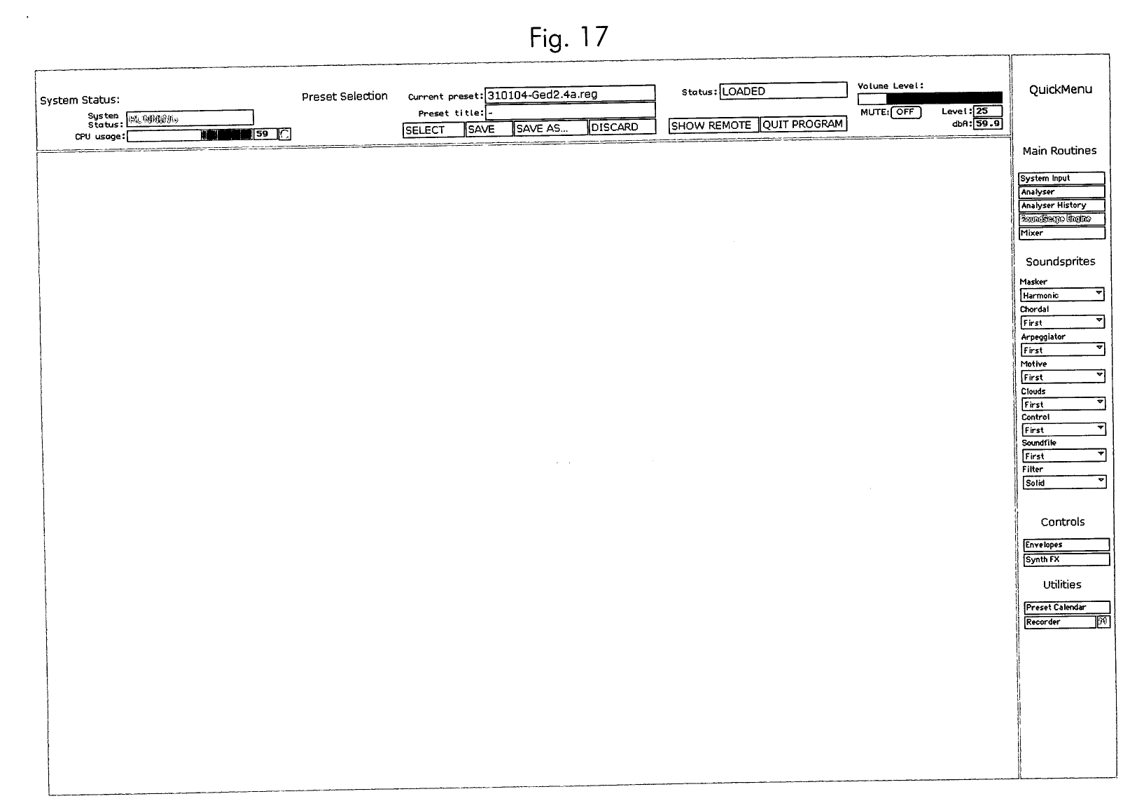

The GUI is shown in FIGS. 17-48. The main control panel is shown in FIG. 17 and remains on the display throughout all of the other windows. The main control panel conveys basic information to the user and lets the user quickly access all the main sub-modules of the system. The information is grouped for display and data entry into logically consistent units. Such groupings include the system status, the preset selection, the volume, main routines, soundsprites, controls and utilities. The system status section includes the system status (running or inactive) and the amount of processor used (CPU usage) in a bar and/or numerical formats. Each bar format shows instantaneous values of the quantity being shown while the graphical formats can show either the instantaneous values or values of the quantity being displayed over a particular time interval. The preset selection section contains the current preset being used and title, if existing, the status of the preset, means to access preset or save/delete a preset, access to a quick controller of the sound screening system, called ‘remote’ and a means to terminate the program. The preset includes settings of the main routines, soundsprites, controls, utilities, and volume. The volume section contains the volume level both in bar and numerical formats (level and dbA) as well as muting control.

The main routine section permits selection of the system input, the Analyser, the Analyser History, the Soundscape Base, and the Mixer. The soundsprites section permits selection of the functional and harmonic maskers, various filters, one or more soundfile soundsprites, Chordal, Arpeggiation, Motive, Control, and Clouds. The controls section permits selection of the envelopes and synthesis effects (named ‘Synth FX’), while the utilities section permits selection of a preset calendar that permits automatic activation of one or more presets and recorder to record information as it is entered to the GUI to create a new preset.

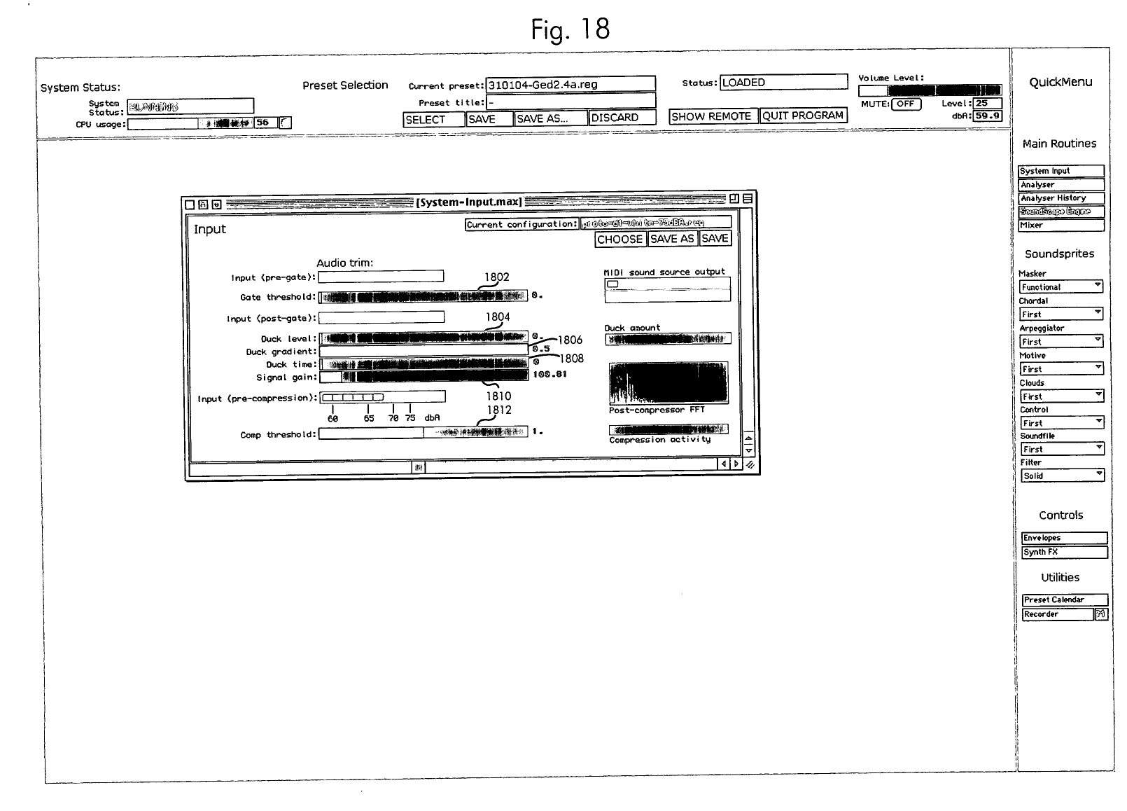

FIG. 18 illustrates the pop-up display that is shown when the system input of the main routine section is selected. The system input pop-up contains a region in which the current configuration is selected and may be altered, and a region in which the different inputs to the system are shown in bar formats, numerical format, and/or graphical format. In fact, as each of the main routines has a current configuration region, such a region for brevity, this feature will not be described in the description of the remaining sections. In an audio trim portion, the gate threshold setting 1802, duck properties (level 1804, gradient 1806, time 1808, and signal gain 1810) and compression threshold 1812 can be set and input levels (pre- and post-gate) and pre-compression input level are shown. The output of the MIDI synthesizer is graphically presented, as are the duck amount post compressor FFT spectrum and compression activity. The user settings set via this interface are saved as part of a specific preset file that can be recalled independently. This architecture allows for the quick configuration of the system for a particular type of equipment or installation environment.

As above, FIG. 19 illustrates the pop-up display that is shown when the Analyser input of the main routine section is selected. The Analyser window is divided in two main areas. The Preset-level controls, which include user parameters which are stored and can be recalled as part of the sound preset (shown in FIG. 16 as ‘sound/response config parameters’) and the remaining area in which parameters are stored as part of a specific configuration file shown at the top of the analyser pop-up window. In a preset-level portion, shown on the left hand side of the pop-up, the gain multiplier 1902, the gate threshold 1904 and the compressor threshold 1906 and multiplier 1908 are set. The input, post-gain and post-gate outputs are displayed graphically. The gain structure and post compressor output are also shown graphically while the final compression activity is shown in a graph, when occurring.

The portion of the Analyser that concerns the main Analysis parameters regarding critical bands and peaks will now be described. In the peak section there is shown peak detection trim and peak event sub-sections. These sub-sections contain numerical and bar formats of the window width 1910 employed in the peak detection process, the trigger height 1912, the release amount 1914, and the decay/sample time 1916, and minimum peak duration 1918 used to generate an event, respectively. These parameters affect the critical band peak analysis described above. The detected Peaks the bar graph on the right of the peak portion. This graph contains 25 vertical sliders, each one corresponding to a critical band. When a peak is detected the slider of the corresponding critical band rises in the graph at a height that corresponds to the energy of the detected peak.

In the portion of the Analyser on the right, user parameters that affect the preset-defined bands are input. A bar graph of the instantaneous output of all of the critical bands is formed above the bars showing the ranges of the four selected RMS bands. The x-axis of the bar graph is frequency and the y-axis is amplitude of the instantaneous signal within each critical band. It should be noted that the x-axis has a resolution of 25, matching the number of the critical bands employed in the analysis. The definition of the preset Bands for the calculation of the preset band RMS values is set by inputs 1920, 1922, 1924 and 1926 which are applied to the bars marked ‘A’, ‘B’, ‘C’ and ‘D’ for the four available preset bands. The user can set the range for each band by adjusting the slider or indicating the low band (starting band) and number of bands in each RMS selection. The corresponding frequencies in Hz are also shown. To the right of the numerical information regarding the RMS band ranges, a history of the values of each of the RMS bands is graphically shown for a desired time period, as is a graph of the instantaneous values of the RMS bands situated below the RMS histories. The RMS values of the harmonic bands based on the center frequencies supplied from the Harmonic Masker 134 are also supplied below the RMS band ranges. The sound screening system may produce a particular output based on the shape of the instantaneous peak spectrum and/or RMS history spectrum shown in the Analyser window. The parameters used for the analysis can be customised for specific types of acoustic environments where the sound screening system in installed, or certain times of the day that the system is in use. The configuration file containing the set parameters can be recalled independently of the sound/response preset and the results of the performed analysis may change considerably the overall response of the system, even if the sound/response preset remains unchanged.

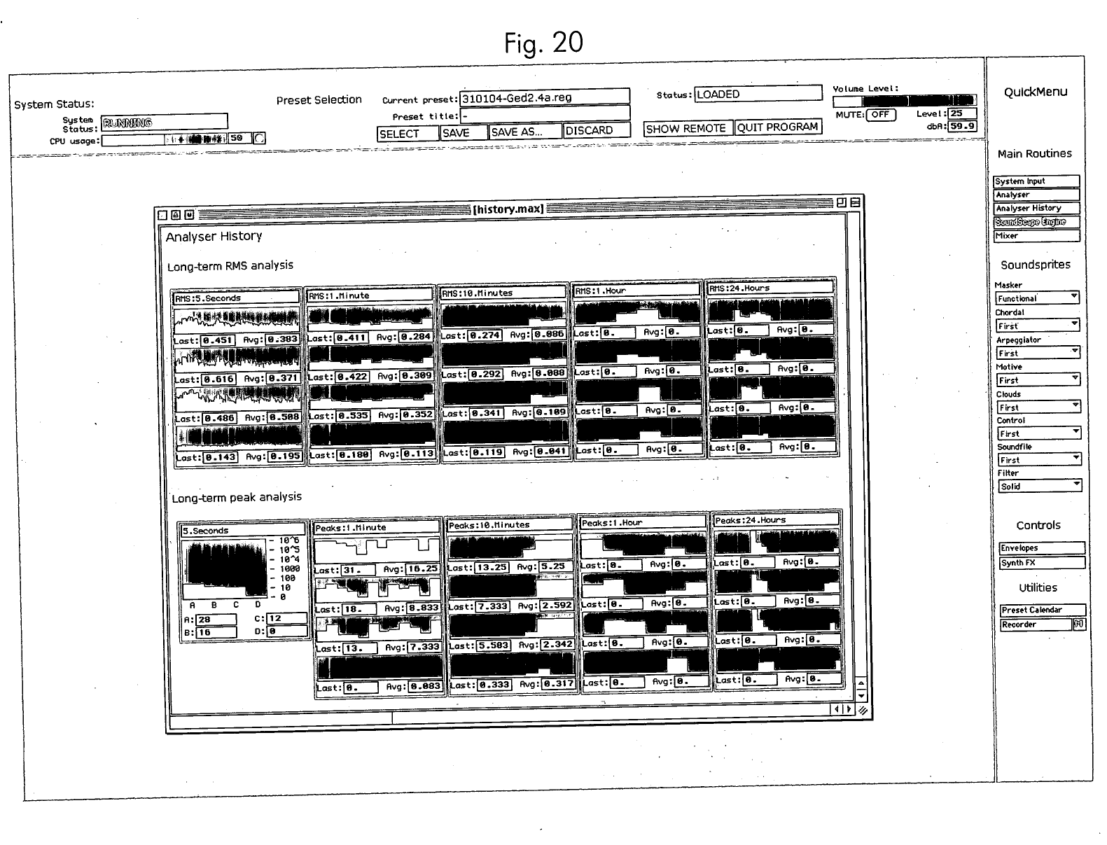

The Analyser History window, shown in FIG. 20, contains a graphical display of the long term analysis of the different RMS and peak selections. As shown, the values of each of the selections (RMS value or number of peaks) are shown for five time periods: 5 seconds, 1 minute, 10 minutes, 1 hour, and 24 hours. As above, these time periods can be changed and/or a greater or less number of time periods can be used. Below each of the graphs are numerical values indicating the immediately preceding value for the last time period and the average value over the total time periods shown in the graph.

The Soundscape Base window, shown in FIG. 21, contains a section for time based settings, named ‘Timebase’, harmonic settings and other controls and a section with pull-down windows showing the unused soundsprites and the different Layergroups. The Timebase section permits the user to change the beats per minute of the system 2102, the time signature of the system 2104, the harmonic density 2106 and the current tonic 2108. These parameters can be automatically adjusted through the Intercom in a way which can be defined through the Intercom settings tab in the Timebase. The harmonic settings section allows user inputs on the probability weightings affecting the Global Harmonic Progression of the System, and the probability weightings affecting the chord selection processes of the various soundsprites. User parameters set for the former are stored in the Global Harmonic Progression Table 2110 and for the latter in four different Tables containing different settings of probability weightings. These are the masterchords 2112 and flexchords1 2114, flexchords2 2116, flexchords3 2118 and flexchords4 2120. The envelopes and synthesizer effects (FX) windows can be launched in the Other Controls section, as can the Intercom connections display shown in FIG. 45. The control section also contains controls for resetting the MIDI Synthesizer 110, including a ‘Panic’ button for stopping all current notes, a Reset Controls and a Reset Volume and Pan Button. The different Layergroups contain soundsprites selected from the unused soundsprites region. By pressing the pull-down menu on the left of the name tab of each soundsprite, the user can select whether the particular soundsprite is off or enabled by being placed on one of the available Layers 1A, 1B, 1C, 2A, 2B, 3A and 3B. When a soundsprite is set to belong to a Layer, it moves to the column of the corresponding Layergroup. Because the soundsprites are listed individually, multiple soundsprites of the same type (e.g. Chordal) may be adjusted independently of each other in the different Layergroups or within a single Layergroup. The information conveyed with each soundsprite thus includes the Layergroup to which the soundsprite belongs, whether the soundsprite has a volume level or is muted, the name of the soundsprite, and the predetermined notes or settings activated by the soundsprite.

The windows containing the settings for the Global Harmonic Progression 2110 and Masterchords 2212 which is one of the five available chord rules used for chord generation are shown in FIG. 22. The Global Harmonic Progression window, on the left hand side of the figure, allows the user to set the parameters affecting the Global Harmonic Progression of the System. The user can set the min/max durations (in beats) 2202a and 2202b for the system to remain at the certain pitch class, if chosen, and the probability to progress to any other pitch class in the multi-slider object 2204 provided. Each bar in the graph corresponds to the probability of the corresponding pitch class shown above to be chosen. Bars of equal height represent equal probability for the selection of either 1, 2b etc. On the right of the multi-slider objects, the min/max duration settings are shown translated in seconds, for the user set values of min/max duration in beats and the Timebase settings. Meanwhile the chord rules (masterchord) window permits the user to set the parameters affecting the chord notes selected for the particular Harmonic Base produced by the Global Harmonic Progression of the system. The user can set the probability weightings manually in the multi-slider object 2208 or select one of the listed chords in the pull-down menu 2206, for example major triad, minor triad, major 7b etc.

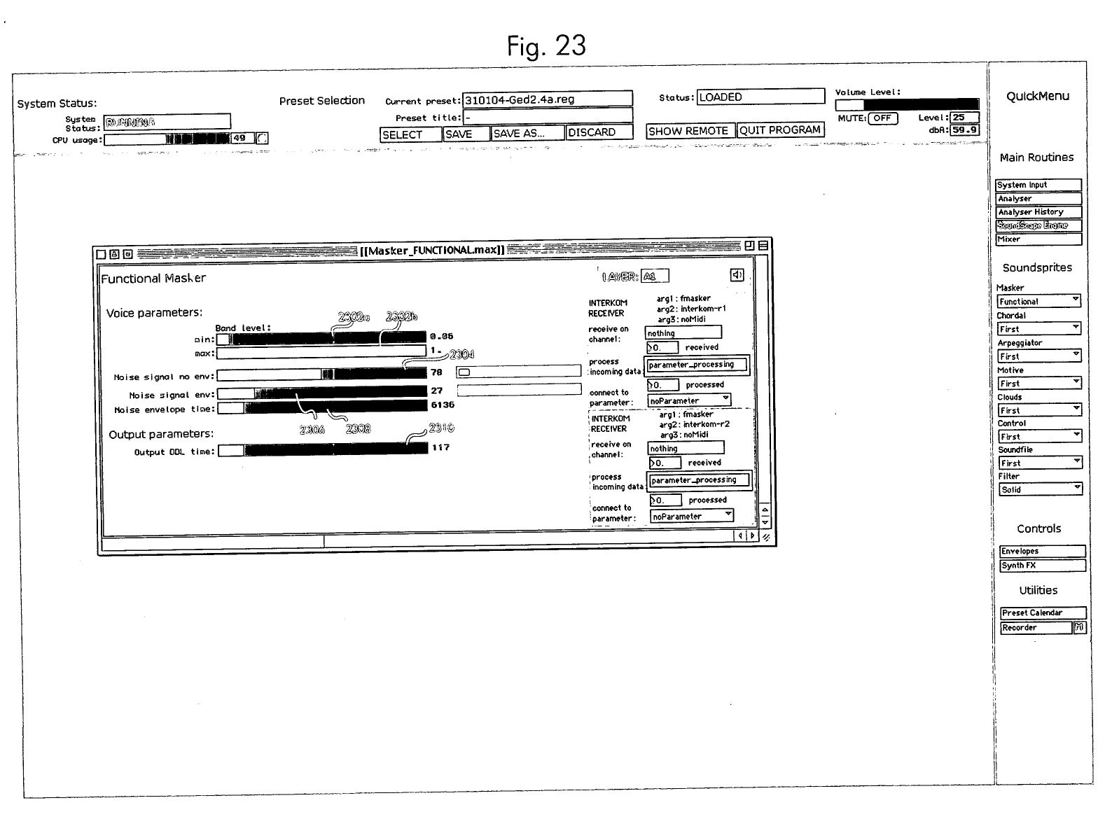

The Functional Masker window shown in FIG. 23 contains a Layer selection, and a mute option section, a voice parameters section, an output parameters section, and sections for different intercom receivers. The voice parameters section allows user control of the minimum and maximum signal levels for each band, 2302a and 2302b respectively, the noise signal level with and without a noise envelope, 2306 and 2304 respectively and the time of the envelope 2308. The output parameters section includes the time for the DDL line 2310. The intercom receivers sections each display the arguments supplied to the particular channel. The reception channel of each of the intercom receivers may be changed, as may the manner in which the received data is processed and the parameter to which the processed received data is then supplied.

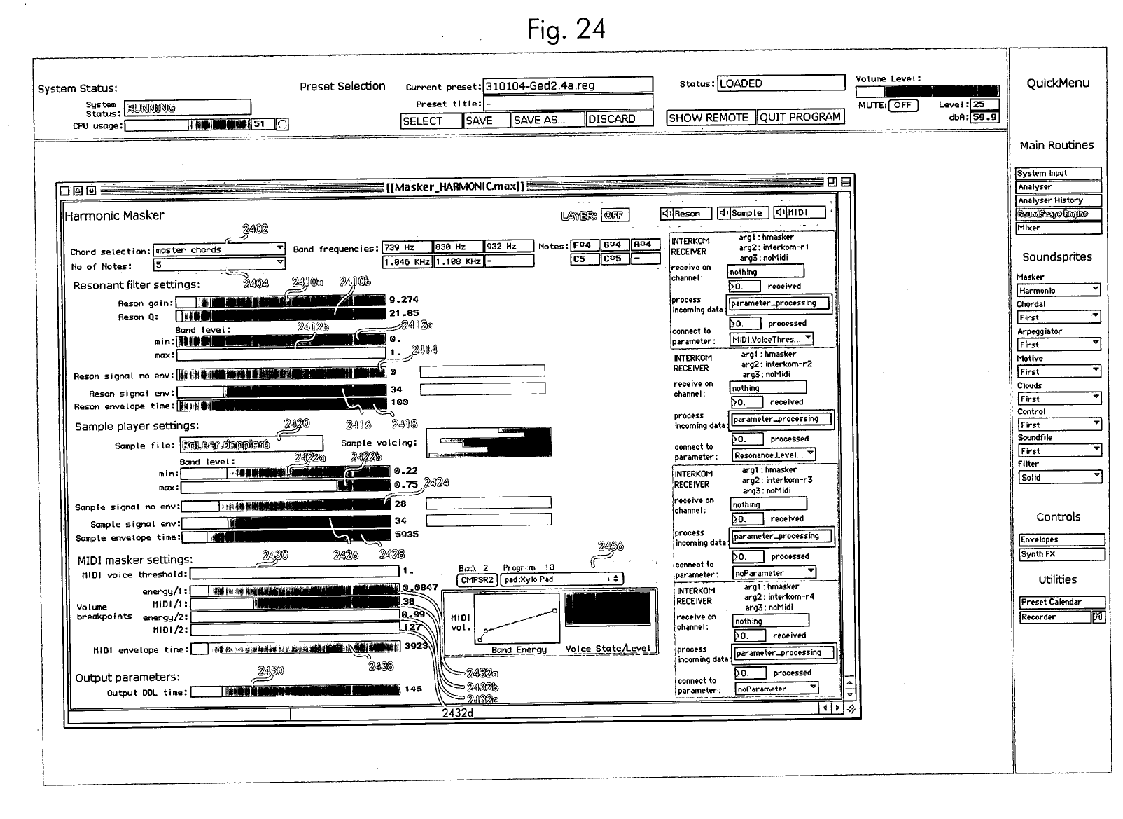

The Harmonic Masker window shown in FIG. 24 contains the same intercom receivers sections as the Functional Masker window of FIG. 23, albeit as shown a greater number of intercom channels are present. Similar to FIG. 23, a Layer selection and mute option section are shown at the top, in this instance providing individual mute options for each type of Harmonic Masker Output. The Harmonic Masker additionally permits adjustment of the chord selection process, including which chord rule to use via user input 2402 and the number of notes to generate 2404. The frequencies in Hz and the notes in MIDI corresponding to the chosen chord members are also displayed. Below this input section are sections displaying the resonant filter settings, sample player settings, MIDI masker settings, and the DDL delay time 2450. The resonant filter settings section contains the gain factor 2410a and the steepness or Q value 2410b of the employed resonant filter, the minimum and maximum signal levels for each band, marked 2412a and 2412b respectively, the resonant signal levels with and without envelope, 2416 and 2414 respectively, and the envelope time 2418 for the latter. The settings are all shown in bar and numerical formats. The sample player settings section contains the activated and alterable sample file 2420, and the minimum and maximum signal levels for each band 2422a and 2422b employed in the Sampleplayer voice, the sample signal levels with and without time envelope, 2426 and 2424 respectively and the envelope time 2428, all shown in bar and numerical formats. The MIDI masker settings shows in bar and numerical formats, the MIDI threshold 2430, multiple volume breakpoints 2432a, 2432b, 2432c and 2432d, and the MIDI envelope time 2438. The volume breakpoints define the envelope shown on the graph on the right of the MIDI masker settings, which defines the MIDI output level for an activated note in relation to the Harmonic Band RMS. The graph on the right named Voice state/level shown the active voices and the corresponding output level. Finally the drop down menus on top of the graphs described allow the user to choose which Bank and which program of the MIDI synthesizer 112 should be employed in the MIDI masker.

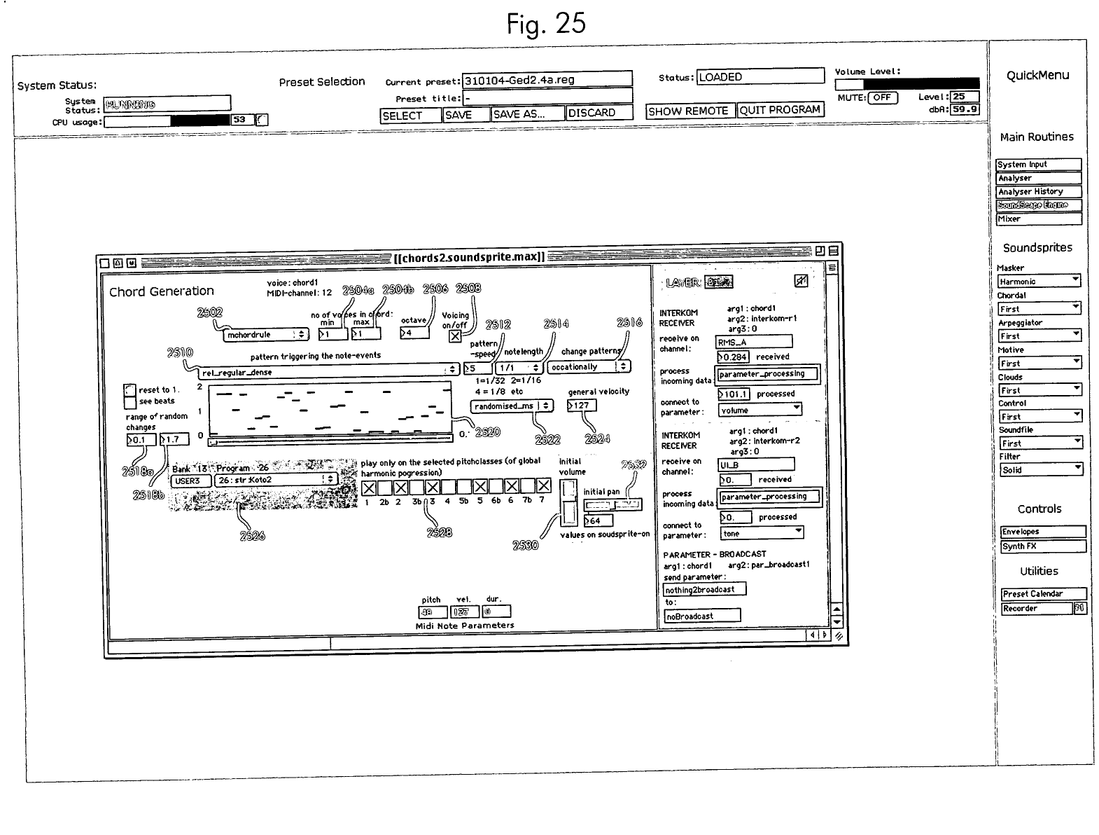

FIG. 25 shows the Chordal soundsprite window. The Chordal soundsprite window has a main portion containing the main generative parameters of the voice and a second portion containing the settings for the Intercom Channels. A pull down menu 2502 for selecting which chord rule to use and number boxes 2504a and 2504b to select a min and max number of notes to be selected are shown at the top of the window. The octave band to which the notes should be transposed can be selected via number box 2506 and Voicing can be turned on or off via the check box 2508. Various pattern characteristics are also entered, such as the pattern list that triggers the note events selected from the drop down menu 2510, the pattern speed (in units of demisemiquavers, i.e. 1/32) which is entered in number box 2512, the length of the notes selected from the drop down menu 2514, and the way in which the pattern is changed selected via the menu 2516. Below the pattern settings, velocity settings can be set. In the graph 2520 shown, the user can set how the velocity of the voice should be changed. The vertical axis corresponds to a velocity multiplier and the horizontal axis to time in beats. The range for the velocity multiplier is set on the left via the number boxes 2518a and 2518b and it can be fixed or be set to automatically change in a pre-described manner selected from the drop-down menu 2522 on the right. The velocity of a note is calculated as the product of the multiplication of the general velocity input on the number box 2524 with the value calculated form the graph 2520 corresponding to the current beat. The input area 2528 is used to select the settings for the pitch Filter of the Chordal soundsprite. Finally the user sets the bank and the program to be used in sub-menu 2526 and the initial volume and pan values via sliders 2530 and 2532 respectively.

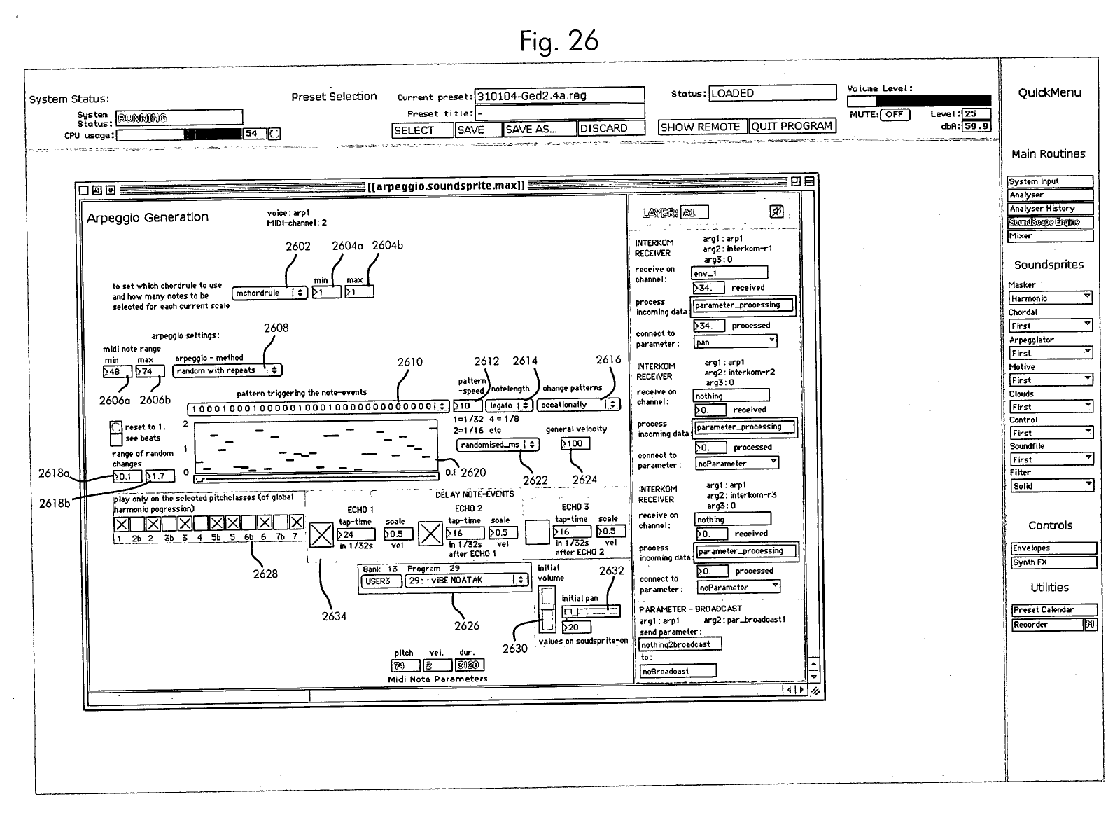

FIG. 26 shows the Arpeggio soundsprite window. As this window accepts many similar settings with the Chordal Soundsprite described above, only the different user settings will be described. Using the number boxes 2606a and 2606b, the user inputs the minimum and maximum MIDI note range, which accepts values from 0 to 127, and the arpeggio method to be used from a pull down menu 2608 containing various methods like: random with repeats, all down, all up, all down then up etc. In the example shown, the random with repeats method has been selected. The user further adjusts the Delay note-events section 2634 which can activate a repeater of the produced notes according to the parameters set.

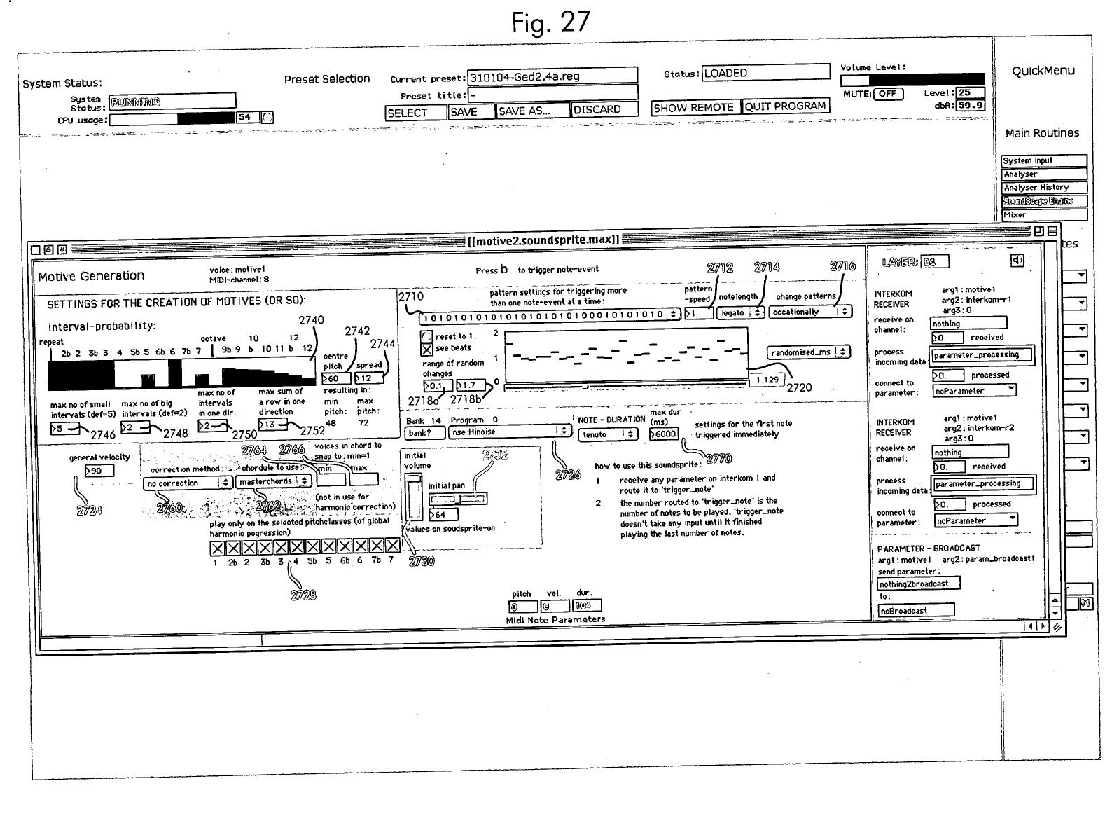

The Motive Soundsprite 156 is shown in FIG. 27. Apart from the settings described above, the user effects settings to control the generation of the motive notes. These are set via the interval probability multi-slider 2740 and the number boxes provided for setting the maximum number of small intervals 2746, maximum number of big intervals 2748, the maximum number of intervals in one direction 2750, the maximum sum of a row in one direction 2752 and the center pitch and spread 2742 and 2744, respectively. Harmonic correction settings are also supplied via the correction method pull down menu 2760, the chosen chord-rule pull-down menu 2762, and the minimum and maximum number of notes to snap in a chord 2764 and 2766, respectively, the latter of which are available only when the correction method is set to ‘snap to chord’. Additionally settings of note duration and maximum note duration are set for adjusting the functionality of the duration filter of the Motive Soundsprite 156.

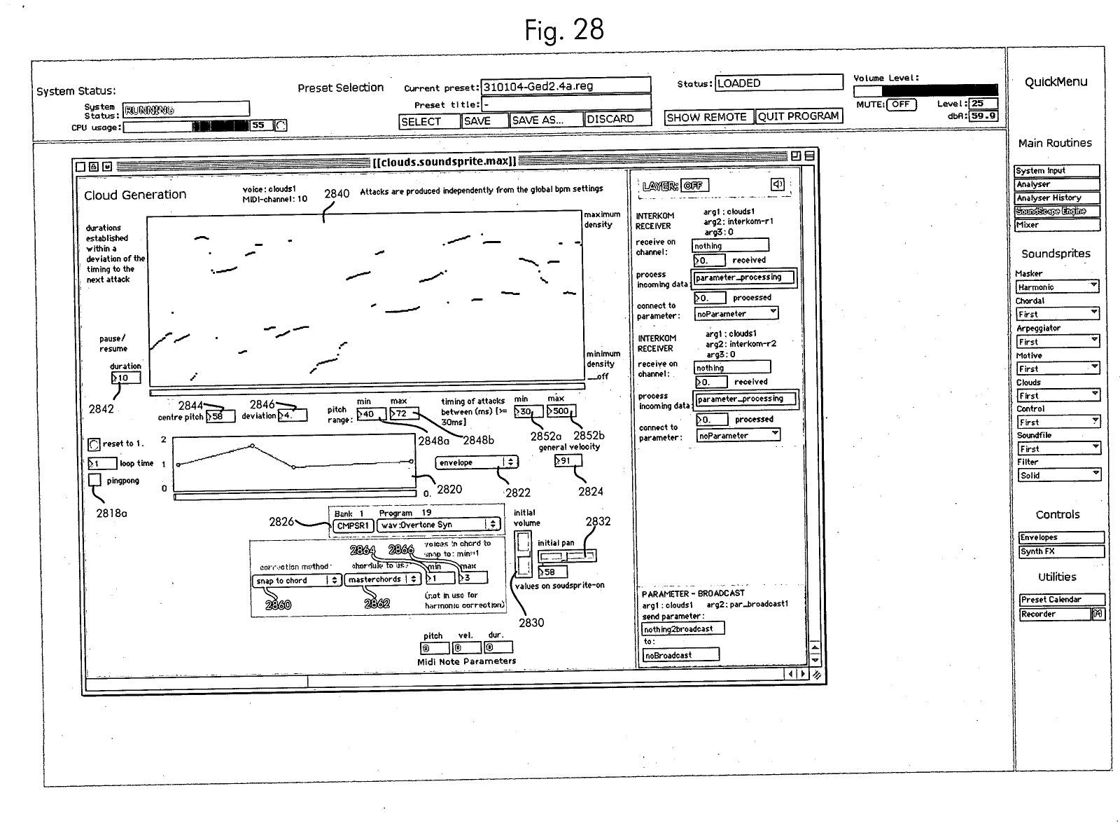

The Clouds Soundsprite 160 is shown in FIG. 28. As discussed in reference to FIG. 13, the pitch and onset generation of the Clouds soundsprite 160 is driven by the settings applied in the multi-slider object 2840. The user draws a continuous or fragmented shape in the multi-slider object 2840 and then sets duration 2842, which is used by the Cloud Voice Generator as the time it takes to scan the multi-slider object along the horizontal direction. For every time instance corresponding to a point on the horizontal axis of the multi-slider object, the value of the graph on the vertical axis is calculated, which corresponds to density of note events generated. High density results in note events generated in shorter time intervals and low density in longer time intervals. The time intervals vary within the range defined via minimum and maximum timing of attacks 2852a and 2852b respectively. The Onset is thus generated via the applied settings described so far. The corresponding pitch values are generated by using a user set center pitch 2844 and a deviation 2846, and are filtered within a defined pitch range between a minimum pitch value 2848a and a maximum pitch value 2848b. The Clouds soundsprite GUI also allows settings for the velocity generation, shown here defined in an alternative graph using user set break points to describe an envelope, harmonic correction and other settings similar to those described for the other soundsprites earlier.

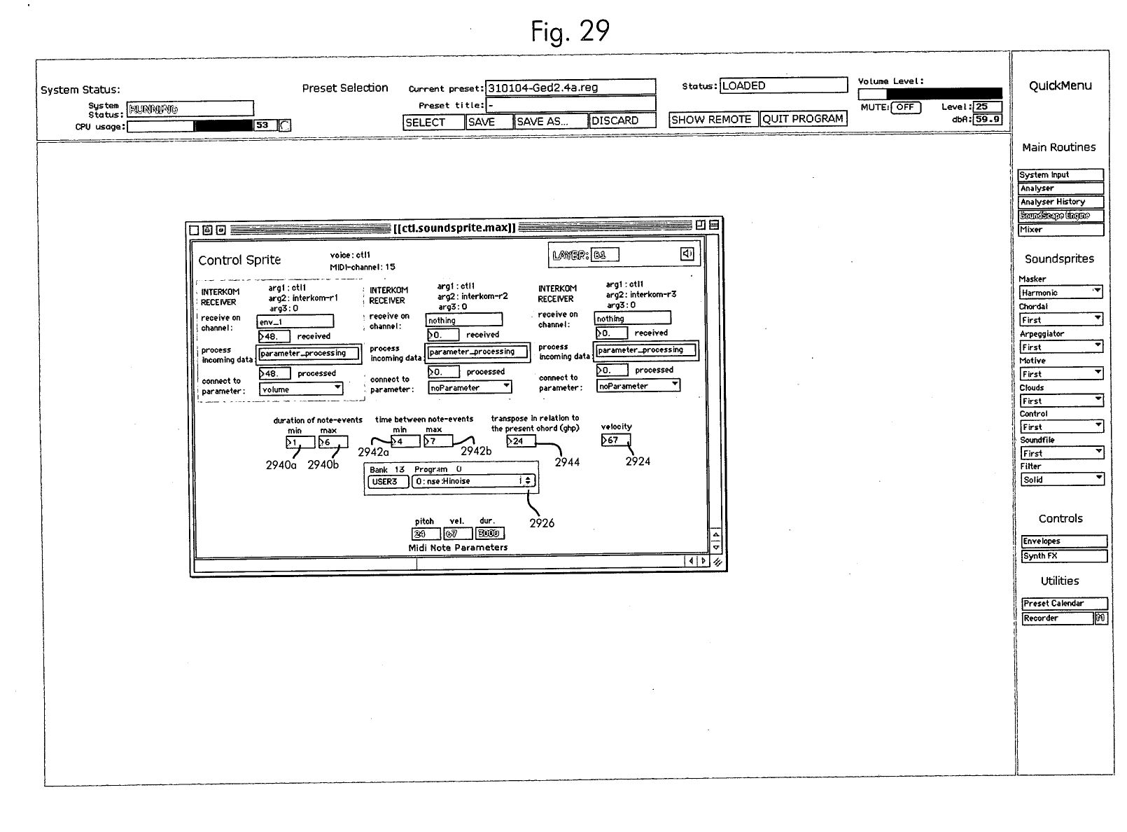

The Control Soundsprite 158 is shown in FIG. 29. The user inputs a minimum and maximum duration of note events to be generated, 2940a and 2940b respectively, the minimum time between note attacks 2942a, a maximum time between note attacks 2942b, a value 2944 representing the amount by which the produced note should be transposed relative to the harmonic base and a velocity setting 2924. The generation of note by the Control Soundsprite also requires setting up a means for regulating the output volume via the intercom. This can be done by acceptance of the data streams available on the local intercom channels and processing it in order to produce volume control MIDI values between 1 and 127.

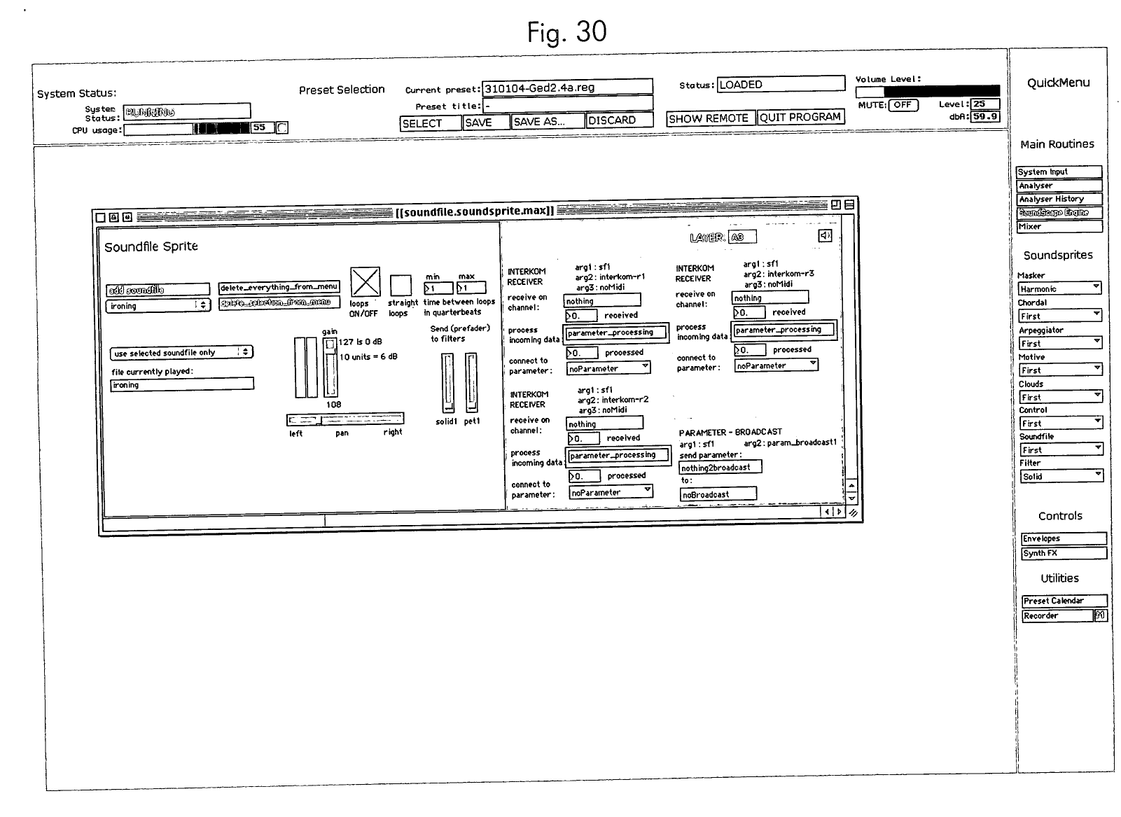

The Soundfile Soundsprite 144 is shown in FIG. 30. This soundsprite also contains a main portion containing the main parameters of operation of the soundsprite and a second portion containing the settings for the Intercom Channels. Controls for selecting one or more soundfiles to be played using Aiff, Wav, or MP3 formats are provided in the main window. Further settings enable the user to select whether one or all of the selected soundfiles should be played in a sequence and whether the selected soundfile or the selected sequence should be played once or repeated in loops. If loops are selected by checking the loops ON/OFF button on the top right side of the main window, time settings are accepted for defining whether the loops are followed by pauses of random duration between a user defined minimum and maximum time periods set by the user in quarterbeats. The gain and pan are also user settable using the provided sliders. There are also options provided to send the soundfile output at a nominal unregulated level to several filters for post processing. By using the available intercom channels, the user can apply settings for automatic adjustment of the output level of the soundfile or soundfiles played, or any of the loop parameters.

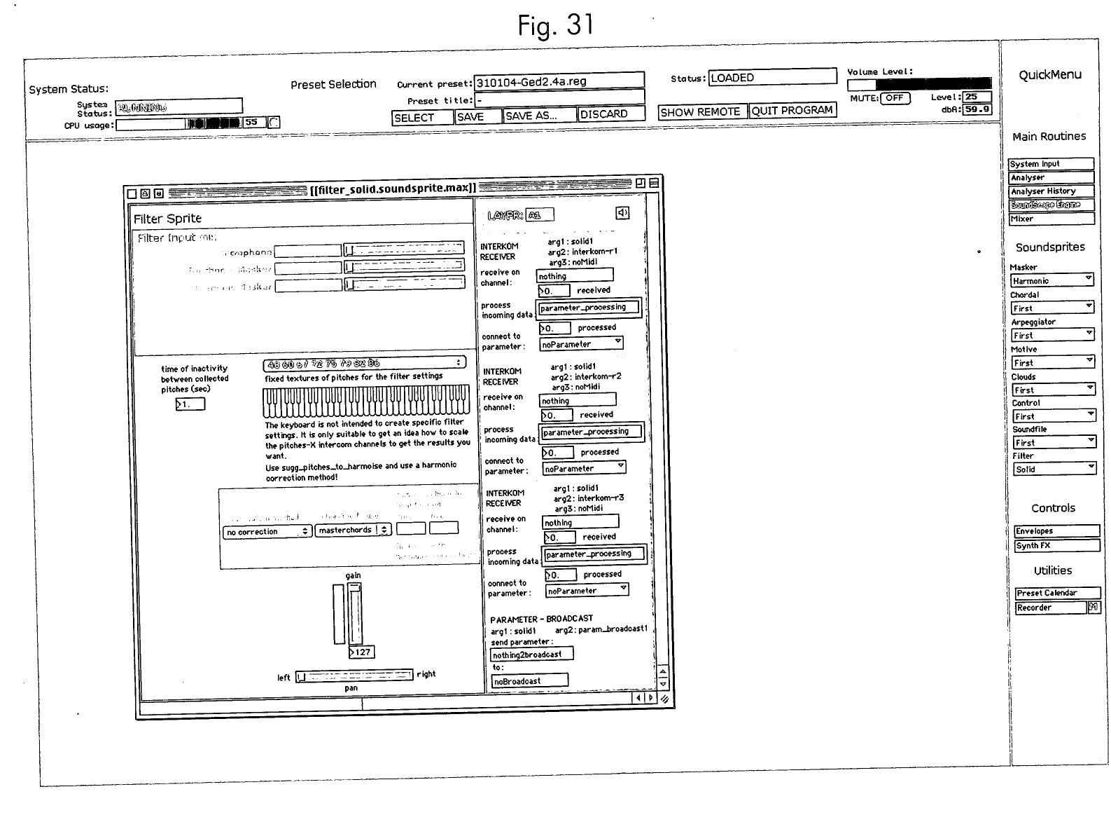

The Solid Filer Soundsprite 136 is shown in FIG. 31. Similar to the soundsprites described above, the GUI for this soundsprite has a main portion containing the main parameters of operation of the soundsprite and a second portion containing the settings for the Intercom Channels. At the top part of the main window, controls for setting the signal levels of the various audio streams available to or from the sound screening system 100 are provided. By adjusting the sliders on the right hand side of the top part of the main window, a user can define which portion of the signal of the microphone 12, the Functional Masker 132, the Harmonic Masker 134, the MIDI Synth 110 and the Soundfile Soundsprites 144 will be passed to the Filtering part of the Solid Filter Soundsprite. On the left part of the Filter Input mix portion of the GUI, the current output levels of the corresponding sources are displayed. In the area below the Filter Input Mix portion of the window, settings are accepted for the selection of the frequencies employed in the filtering process. The user can select one of the fixed frequency sets provided as lists of pitches in a drop-down menu, or use the intercom to define pitches in relation to data broadcasted by the analyser. When the latter option is exercised, the user can further define the parameters of a harmonic correction method to be used for filtering the suggested pitches. Further user controls are also provided for setting the filter gain and pan and setting up the appropriate relations via the Intercom.

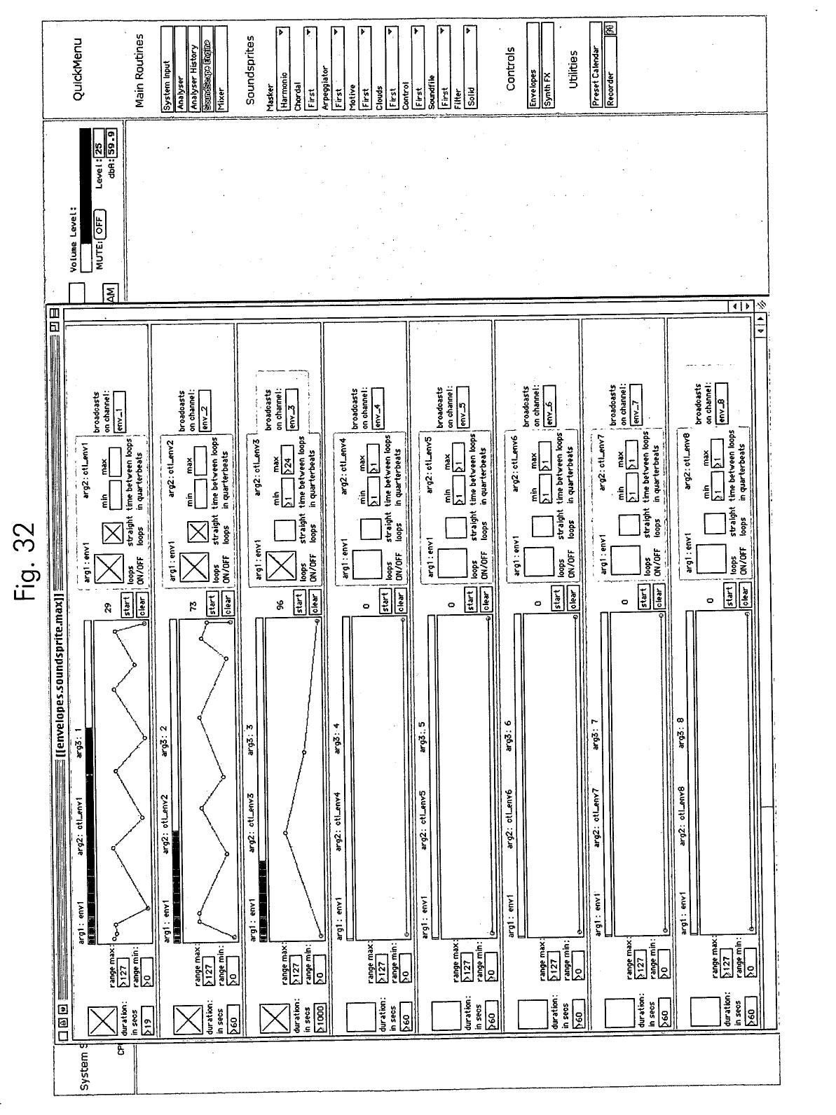

The Envelopes soundsprite of the main control panel of FIG. 17 is shown in FIG. 32. The Envelopes soundsprite window contains settings for defining multiple envelopes, used to produce continuous user-defined streams of integer values, which are broadcast over dedicated Intercom channels. The user first selects the duration of the stream and the range of the values to be produced and then shapes an envelope in the corresponding graphical area by adjusting an arbitrary number of points which define a line. The height of the drawn line for any time instance between the start and the defined duration corresponds to a value between the minimum and maximum values of the range set by the user. Shown on the right are user selectable options for repeating the value stream once it has ended, with options provided for straight loops to be produces or loops separated by a pause the duration of which is randomly selected between a minimum and a maximum time set in seconds by the user. The value-streams generated are broadcast through the intercom over dedicated channels env_1 to env_8.

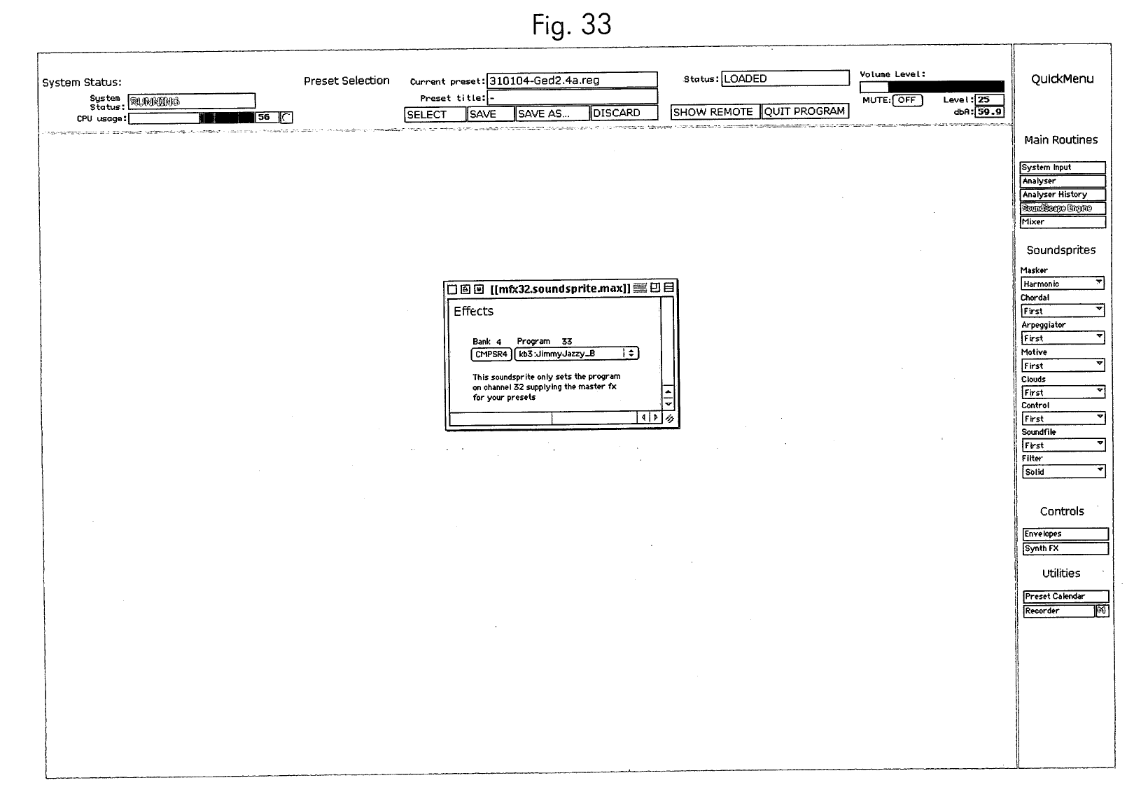

The GUI for the Synth Effects Soundsprite 174 is shown in FIG. 33. Settings are provided to the user for selecting the Bank and Program of the Midi Synth 110, which supplies the master effects for all the MIDI output of the sound screening system.

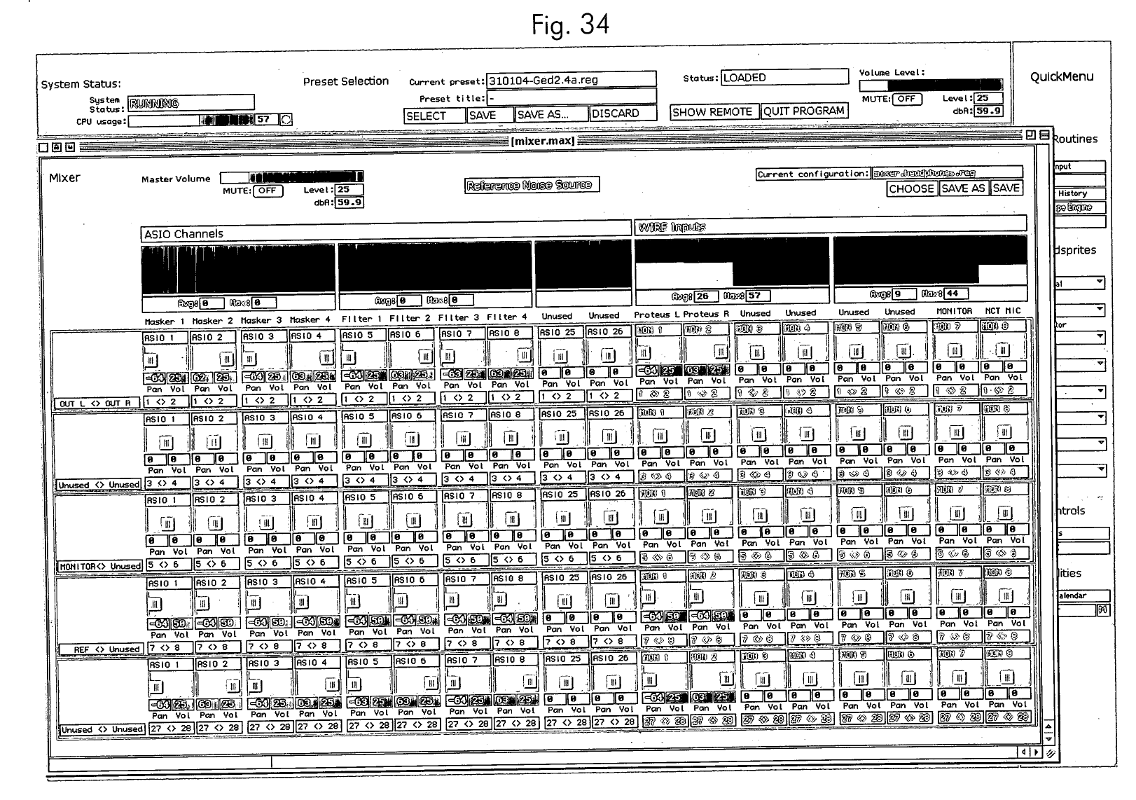

The Mixer window shown in FIG. 34 has a section in which the user can choose the configuration or save a current configuration. The volume control of the Mixer output is shown to the right of the configuration section in both numerical input and bar format. Below these sections the audio stream input/output (ASIO) channels and wire inputs are shown. The average and maximum of each of the ASIO channels and wire inputs are shown. The ASIO channels and wire inputs contain settings that, as shown, are graphical buttons that may be slid to establish the volume control. The ASIO channels have settings for the four masker channels and four filter channels and the wire inputs have settings for a microphone and other connected electronics such as a Proteus synthesizer. The left and right channels to the speaker are shown below each of the settings.

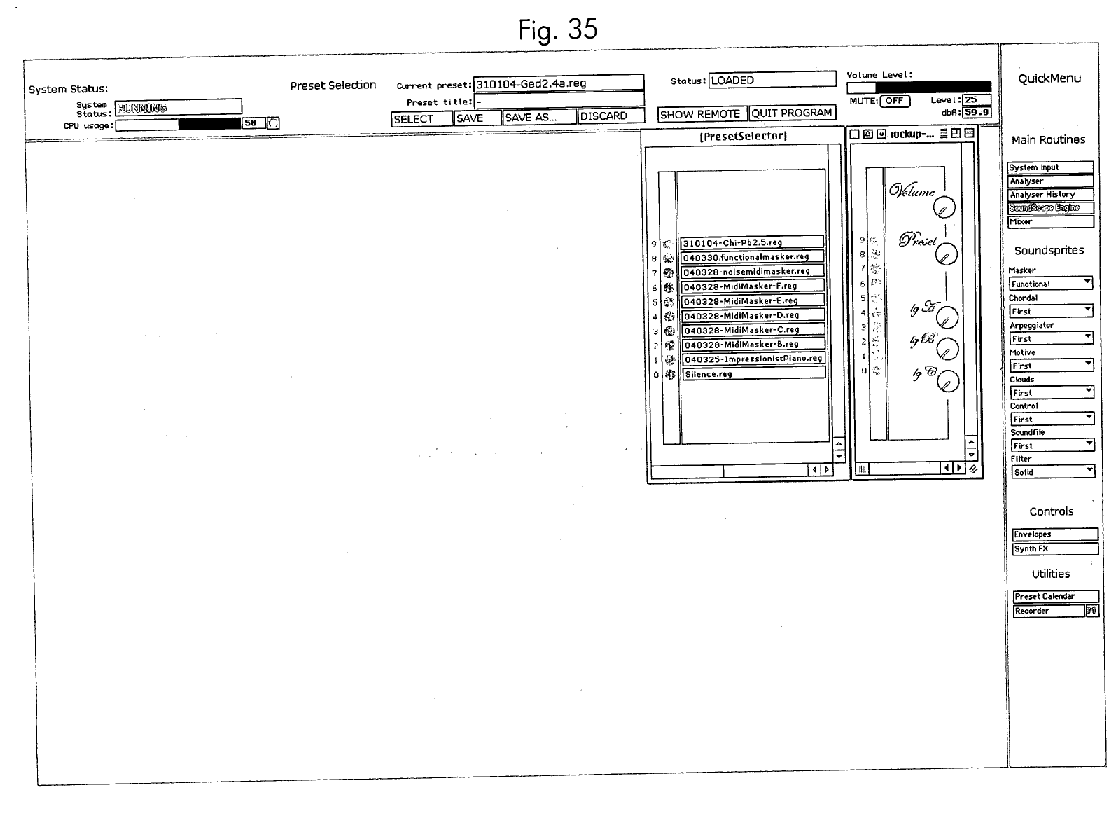

FIG. 35 shows a Preset Selector panel of the GUI selected via the ‘show remote’ button of the GUI shown in FIG. 17. A pop-up window allows selection of a particular set of presets loaded in the selected positions 0-9 of the Preset-Selector Window. The Pop-up window on the right contains dials for quickly changing key response parameters of the sound screening system 100, including the volume, the preset and three LayerGroup Parameters assigned to specific parameters within the system via the intercom. By adjusting the Preset dial, the user selects a value from 0 to 9 and the corresponding preset selected on the pop-up window on the left is loaded. This interface is an alternative interface for controlling the response of the sound screening system. In some embodiments, a separate hardware controller device of the same layout with the graphical controller shown on the pop-up window on the right can be used as a controller device communicating with the graphical controller via a wired or wireless connection.

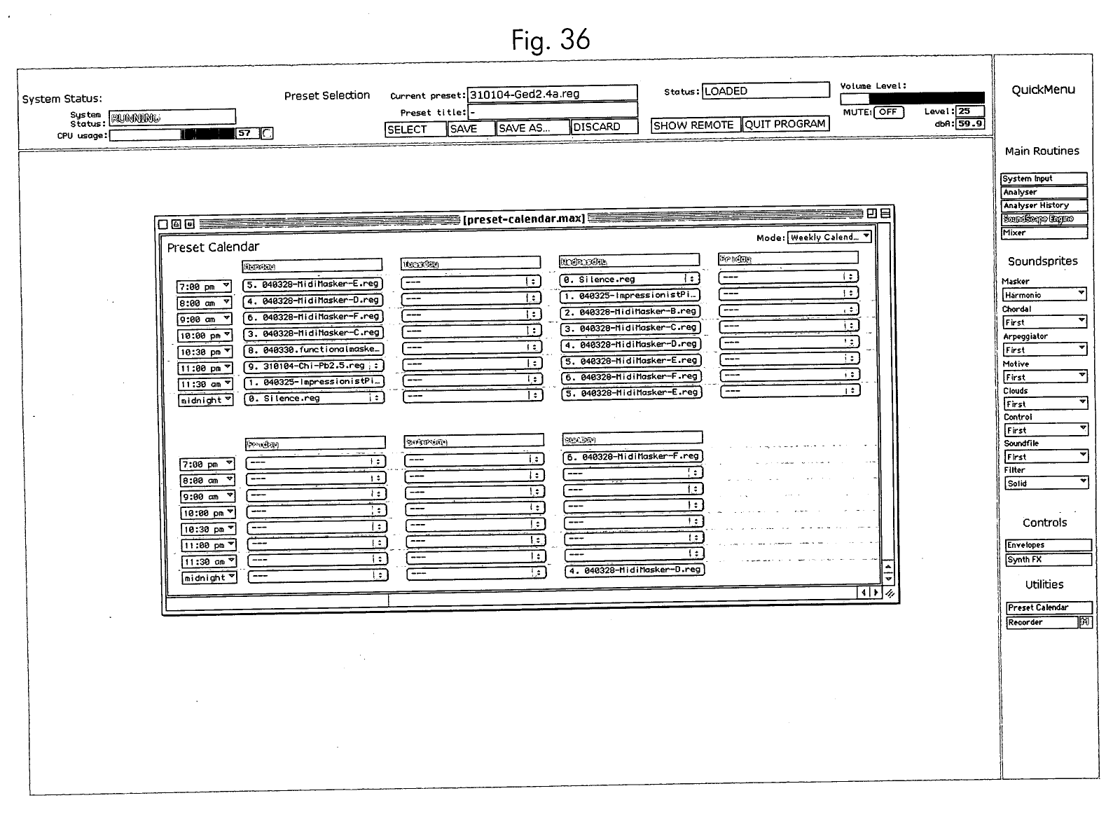

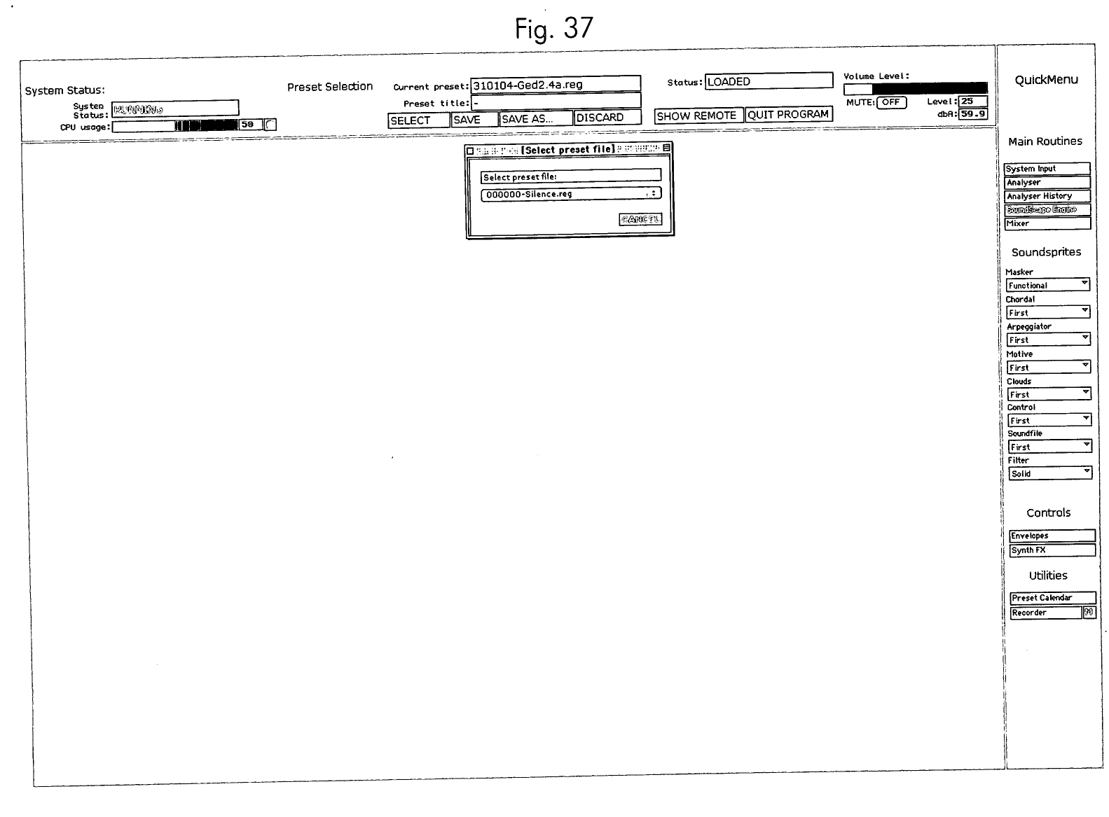

The Preset Calendar window of FIG. 36 permits local and remote users to choose different presets for different periods of time over a particular calendar period. As shown, the calendar is over a week, and the presets are adjusted over the course of a particular day. FIG. 37 shows typical Preset Selection Dialog Boxes in which a particular preset may be saved and/or selected.

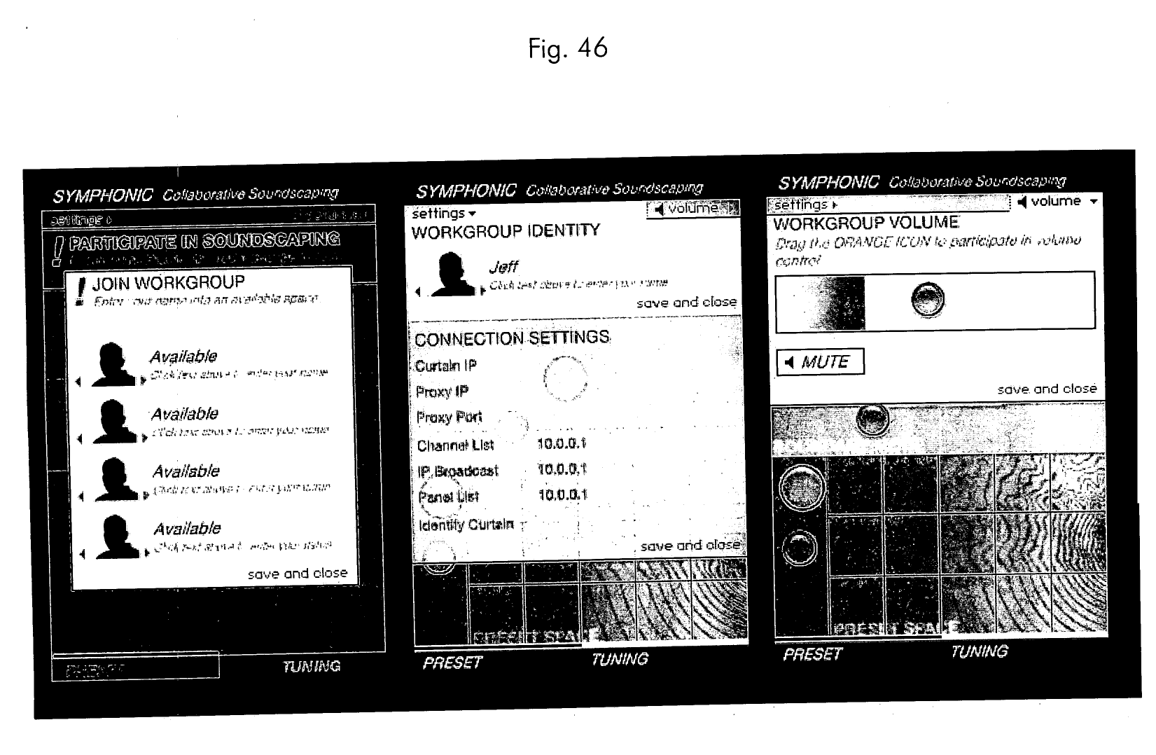

FIGS. 46-48 show one embodiment of a system that permits shared control of one or more sound screening systems over the LAN. At the user end, the control interface is accessible via a web browser on a computer, personal digital assistant (PDA), or other portable or non-portable electronic device capable of providing information between the interface and the sound screening system. The control interface is updated with the information on the current state of the system. The user is able to affect the state of the system by inputting the desired state. The interface sends the parameter over to the local system server, which either changes the state of the system accordingly, or uses it as a vote in a vote-by-proximity response model. For example, the system will respond solely to a user if the user has master control of the system or if no other users are voting.

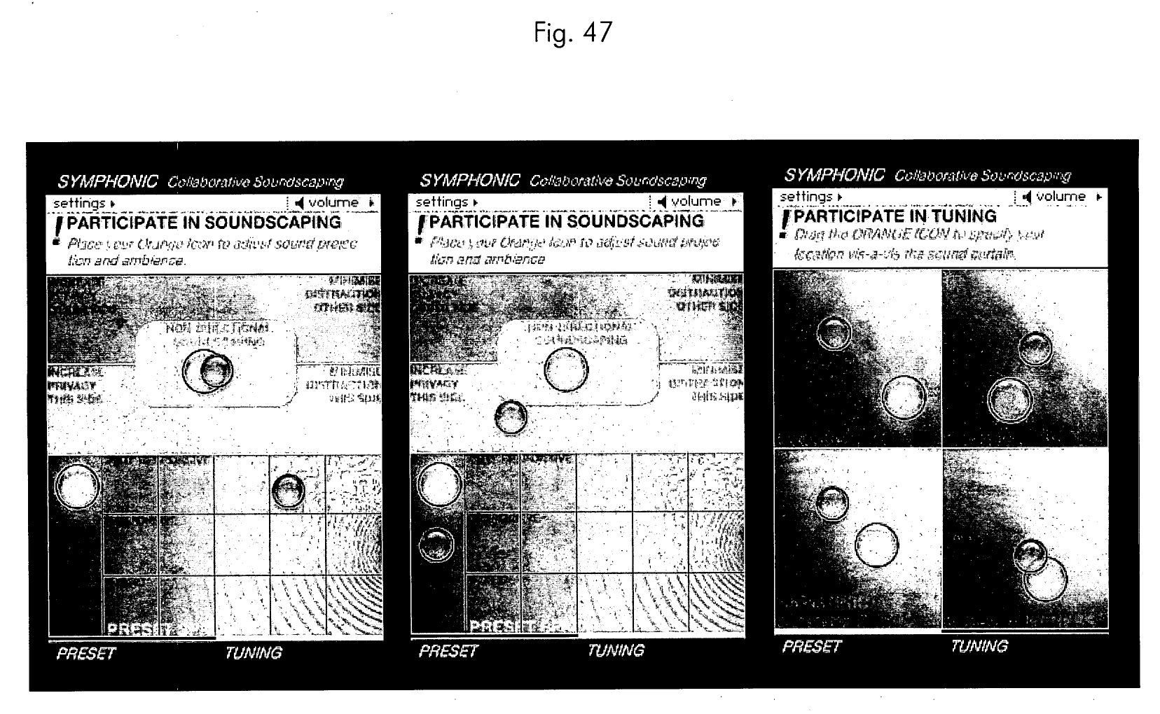

In FIG. 46, multiple windows are shown in a single screen of the GUI. The leftmost window permits a user to join a particular workgroup ‘owning’ one or more sound screening systems. The user identity and connection settings for IP addresses used by the LAN are provided in a second window. A third window allows the user to adjust the volume of sound from the sound screening system using icons. The user can also set the sound screening system to determine how responsive it is to external sounds incident upon it. As shown in FIG. 47, the user can further tailor the effects of each sound screening system controlled to his or her personal preference through the graphical interface and icons. As shown, the projection of the sound from the sound screening system and ambiance on different sides of the screen can be regulated by the user. Accordingly, the soundscaping can be non-directional, can be adjusted to increase the privacy on either side of the sound screening system, or can be adjusted to minimize distractions from one side to another. Besides the responsiveness to external sounds, a user can also adjust various musical aspects of the response, such as colour, rhythm, and harmonic density. In these figures, the current response of the system is shown by the larger circles while the user enters his/her preference by dragging the smaller circles into the desired locations.

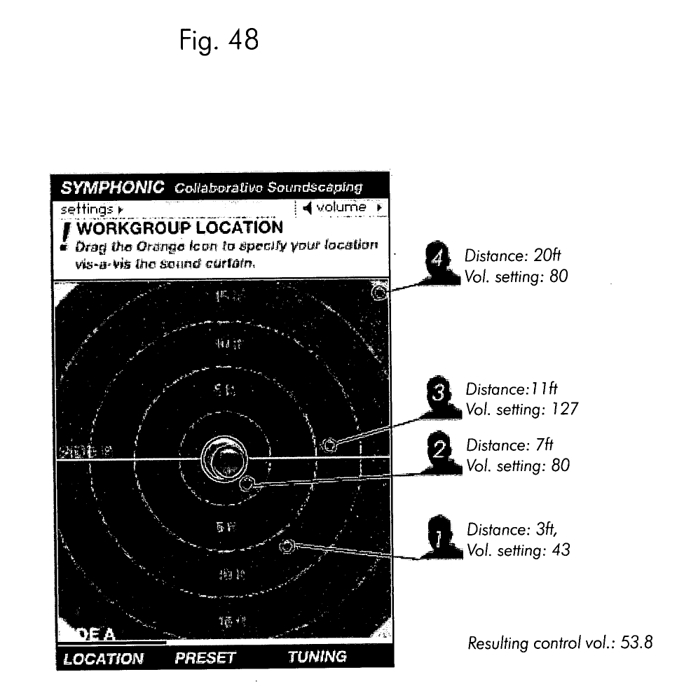

FIG. 48 illustrates one manner by which the response of the sound screening system is modified by multiple users, i.e. proximity implementation takes place. In this method, the amount of weight that is given to the vote of a particular user is inversely proportional to the distance of the user from the sound screen. Each user thus enters his or her distance as well as direction from the sound screen as shown in the figure.

More specifically, as shown, if N users of distance Rᵢ (for the ith user) from the sound screen are logged into the system and vote on a particular characteristic of the sound screening system (such as volume from the sound screening system), then the value of the characteristic is:

$$X = \frac{{\sum }_{i = 1}^{N} \frac{{X}_{i}}{{R}_{i}^{2}}}{{\sum }_{i = 1}^{N} \frac{1}{{R}_{i}^{2}}}$$

In other embodiments, the directionality of the users as well as distance may be taken into account when determining the particular characteristic. Although only about 20 feet is illustrated as the range over which the user can have a vote, this range is only exemplary. Also, other weighting schemes may be used, such as a scheme that takes into account the distance differently (e.g. 1/R), takes into account other user characteristics, and/or does not take into account distance altogether. For example, a particular user may have an enhanced weighing function because he or she has seniority or is disposed in a location that is affected by sounds from the sound screening system to a larger extent than other locations of the same relative distance from the sound screen.

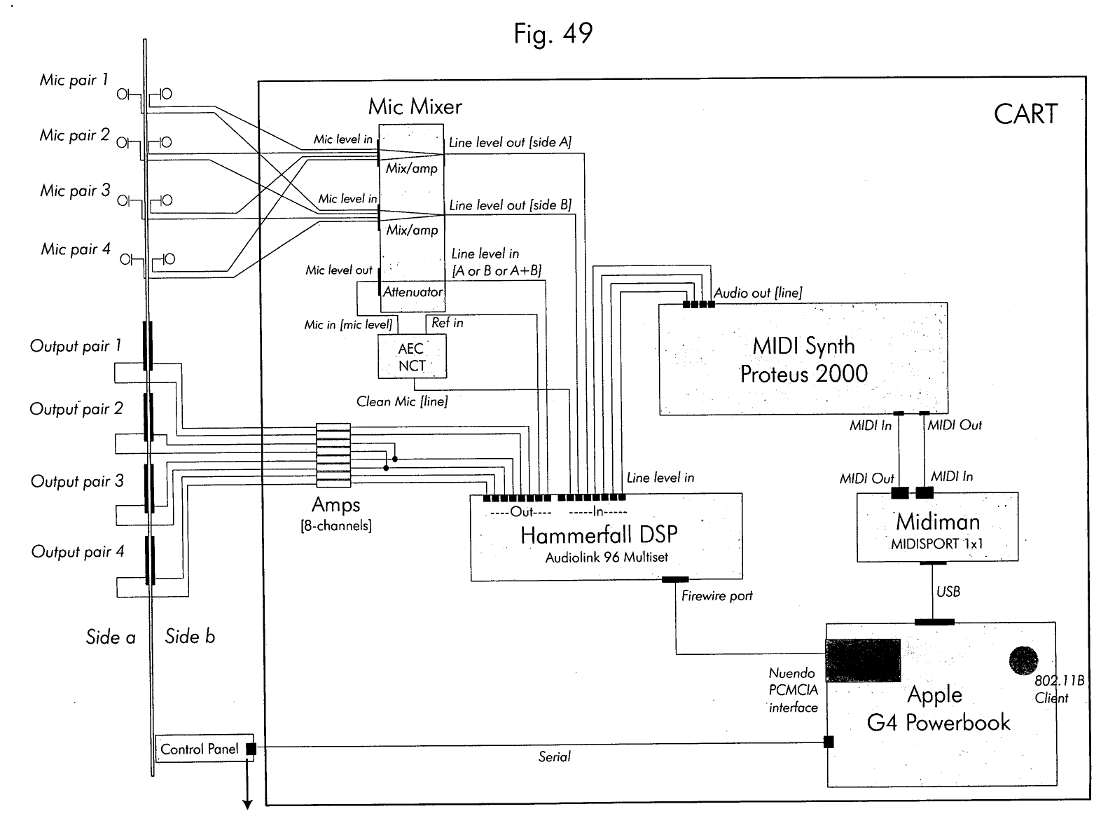

The physical layout of one embodiment of the sound screening system as well as communication between the user and the sound screening system(s) will now be described in more detail. FIG. 49 shows a sound screening system employing several hardware components and specifically written software. The software, running on an Apple PowerBook G4, is written in Cycling'74's Max/MSP, together with some externals written in C. The software interfaces to a Hammerfall DSP audio interface via an ASIO interface, and it also controls the Hammerfall's internal mixer/router using a Max/MSP external. The software also drives one or two Proteus synthesisers via MIDI. External control is done using a physical control panel with a serial interface (converted to USB for the PowerBook), and there is also a UDP/IP networking layer to allow units to communicate with each other, or with an external graphical interface program. The system receives input from the sound environment using an array of sound sensing components routed to the Hammerfall DSP audio interface via a Mixer and an Acoustic Echo Cancellation Unit supplied by NCT. The response of the system is emitted into the sound environment by an array of sound emitting units interfacing with the Hammerfall DSP via an array of Amplifiers.

The sound screening system also employs a physical sound attenuating screen or boundary on which the sound sensing and sound emitting components are placed in such a way that they effectively operate primarily on the side of the screen or boundary on which they are positioned. The input components can be, for instance, hypercardiod microphones mounted in pairs at a short distance, for example 2 inches, over the top edge of the screen and pointing to opposite directions, so that the one is picking up sound primarily from the one side of the screen and the other from the opposite side of the screen. As another example, the input components can be omnidirectional microphones mounted in pairs in the middle but opposite sides of the screen. Similarly, the output components can be, for instance, pairs of speakers, mounted on opposite side of the screen, emitting sound primarily on the side of the screen on which they are placed.

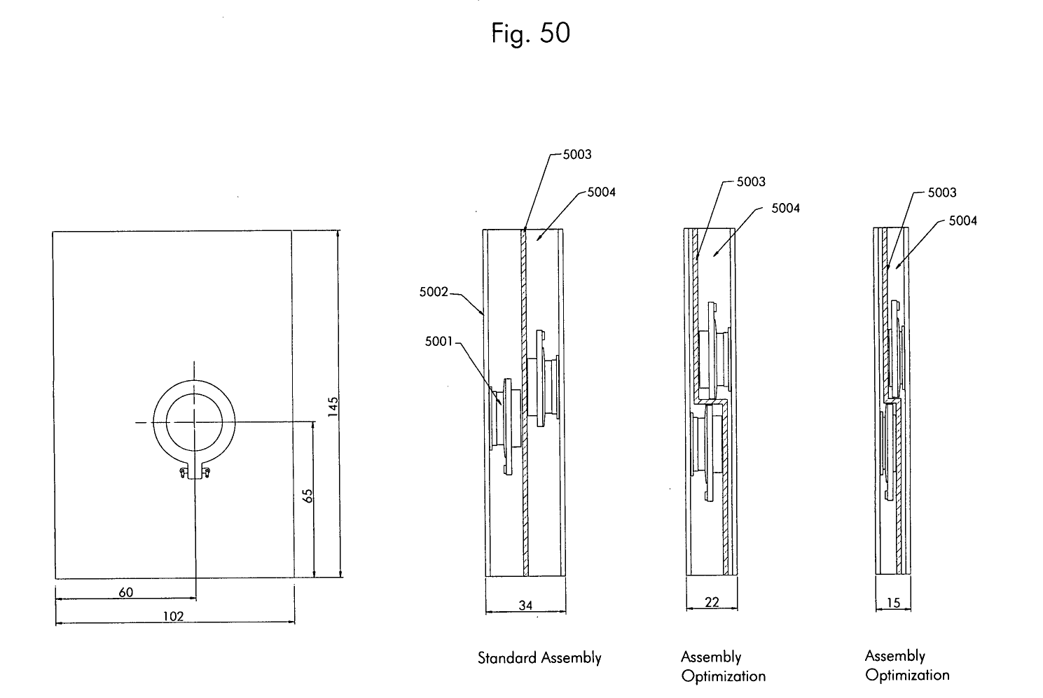

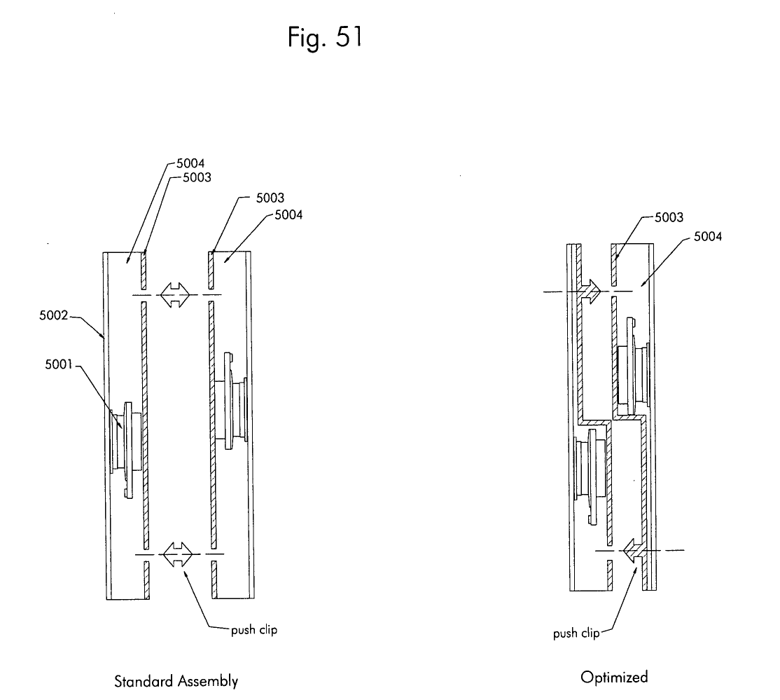

In one embodiment, the speakers employed are flat panel speakers assembled in pairs as shown in FIG. 50 and FIG. 51. In the figures, a flat panel speaker assembly contains two separate flat panel speakers separated by an acoustic medium 5003. A panel 5002 is selected from a suitable material, like an 1 mm thick ‘Lexan’ 8010 polycarbonate supplied by GE plastics and is has a size of 200×140 mm. The panel 5002 is excited in audible vibration using an exciter 5001 like the one supplied from NXT having a 25 mm diameter and 4 ohm resistance. The panel 5002 is suspended along its perimeter using a suspension foam, like a 5 mm×5 mm double-sided foam supplied by Miers, on a frame constructed of a rigid material like a 8 mm Grey PVC which is mounted on an acoustic medium 5003 made for example from a 3 mm polycarbonate sheet. The gap between the acoustic medium 5003 and the panel 5002 can be filled with acoustic foam 5004 like a 10 mm thick melamine foam to improve the frequency response characteristics of each speaker monopole.

As shown in FIG. 50, the acoustic medium 5003 may be substantially planar, in which case the exciters 5001 disposed on opposite sides of the acoustic medium 5003 do not overlap in the lateral direction of the flat panel speaker assembly (i.e. the direction perpendicular to the thickness direction indicated by the double ended arrows). Alternately, the acoustic medium 5003 contains one or more perpendicular bends forming, for example, an S-shape. In this case, the exciters 5001 disposed on opposite sides of the acoustic medium 5003 overlap in the lateral direction.

As shown in FIG. 51, the arrangements of FIG. 50 can be assembled as a single unit with only one acoustic medium 5003 between the exciters 5001, or multiple units can be snap-fitted together using one or more push clips. Each unit contains one or more exciters 5001, the panel 5002 on one side of the exciter 5001, the acoustic medium 5003 on an opposing side of the exciter 5001 and acoustic foam 5004 disposed between the panel 5002 and the acoustic medium 5003. The units may be snap-fitted together such that the acoustic medium 5003 contact each other.

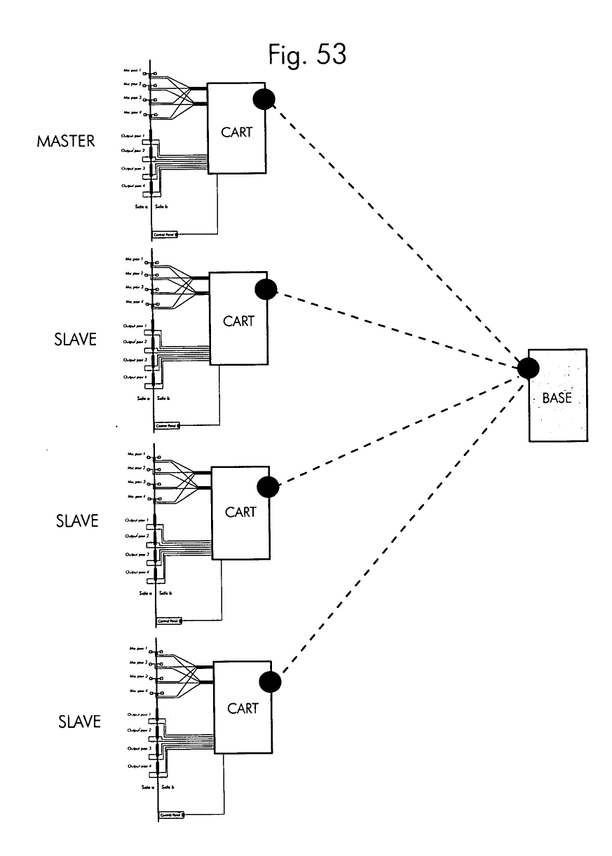

The sound screen (also called curtain) can be formed as a single physical curtain installation of any size. The sound screening system has a physical controller (with indicators such as buttons and/or lights) and one or more “carts” containing the electronic components needed. In one implementation, as shown in FIG. 49, a cart contains a G4 computer plus network connection and sound generating/mixing hardware. Each cart has an IP address and communicates via wireless LAN to a base and to other carts. Every operating unit, comprising of one or more cart, has a cart named as ‘master’. Such a unit is shown in FIG. 53. Larger units have one or more carts named ‘slaves’. A cart may communicate to other carts in the same unit, or potentially to carts in other units. Carts communicate using any language desired, such as an open source code (OSC). A base is, for example, a computer with a wireless LAN base station. The base computer runs the user interface (Flash) and an OSC proxy/networking layer to talk to all the carts in the unit that the base is controlling. In one embodiment, most of the intelligence in the base is in a Java program which mediates between the Flash interface and the carts, and also manipulates the curtain states according to entries in a database. Every cart, and every base, is configured with a static IP address. Each cart knows (statically) the IP address of its base, and its position within a unit (master cart, or some slave cart), and the IP addresses of other carts in the unit.

The base has a static IP address, but does not know anything about the availability of the carts: it is the responsibility of the carts to periodically send their status to the base. The base does, however, have a list of all possible carts, since the database has a table of carts and their IP addresses, used for manipulating the preset pools and schedules. Different modes of communication may be used. For example, 802.11B communication may be used throughout if the carts use G4 laptops which have onboard 802.11B client facilities. The base computer can be equipped with 802.11 B also. The base system may be provided with a wireless hub.

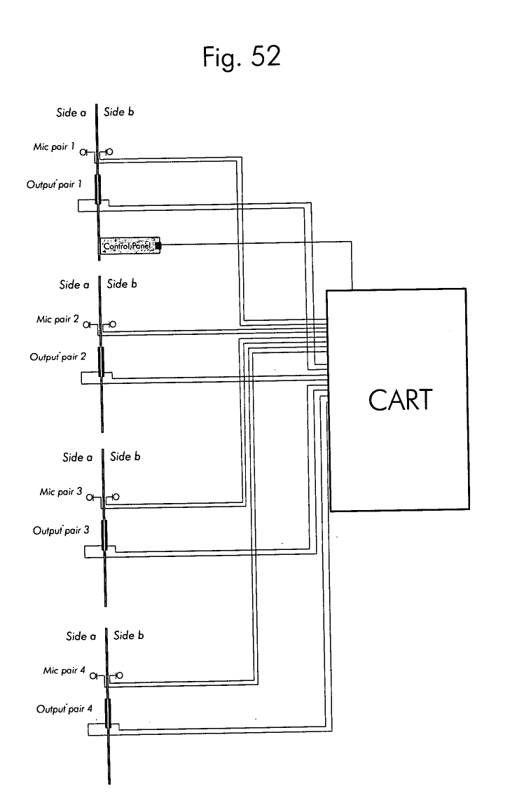

The curtain may be a single physical curtain with a single cart that has, for example, four channels. Such is the system shown in FIG. 49. This configuration is known as an individual system and is standalone. Alternately, multiple curtains (such as four curtains) can work together with a single cart that has the four channels, as shown in FIG. 52. This configuration is known as a workgroup system and is standalone. In addition, multiple curtains can work together in multiple carts with twelve or sixteen channels and using a base, as shown in FIG. 53. This configuration is known as an architectural system.

The software components of the base can consist of, for example, a Java network/storage program and a Flash application. In this case, the Flash program runs the user interface while the Java program is responsible for network communications and data storage. The Flash and Java programs can communicate via a loopback Transmission Control Protocol (TCP) connection exchanging Extensible Markup Language (XML). The Java program communicates with curtain carts using open sound code (OSC), via user data protocol (UDP) packets. In one embodiment, the protocol is stateless over and above the request/reply cycle. The data storage may use any database, such as an open source database like MySQL, driven from the Java application using Java Database Connectivity (JDBC).

Operation of the software may be either in standalone mode or in conjunction with a base, as discussed above. The software is able to switch dynamically between the two modes, to allow for potential temporary failures of the cart-to-base link, and to allow relocation of a base system as required.

In standalone mode, a system may be controlled solely by a physical front panel. The front panel has a fixed selection of sound presets in the various categories; the “custom” category is populated with a selection of demonstration presets. A standalone system has a limited time sense: a preset can change its behaviour according to time of day or, if desired, a sequence of presets may be programmed according to a calendar. The front panel cycles along presets in response to button presses, and indicates preset selection using on-panel LEDs.

In (base) network mode, the system is essentially stateless; it ignores its internal store of presets and plays a single preset which is uploaded from the base. The system does not act on button presses, except to pass the events to the base. The base is responsible for uploading presets, which the system must then activate. The base also sends messages to update the LEDs on the display. The system degrades operation gracefully on network failure; if the system loses its base, it continues in standalone mode, playing the last preset uploaded from the base indefinitely, but activating local operation of its control panel.

The communication protocol between the base and the cart is such that all requests, in either direction, utilise a simple handshake, even if there is no reply data payload. A failure in the handshake (i.e. no reply) may re-trigger a request, or be used as in indication of temporary network failure. A heartbeat ping from the base to the cart may exist. This is to say that the base may do periodic SQL queries to extract the IP addresses of all possible systems and ping these. New presets may be uploaded and a new preset activated, discarding the current preset. The LED status would then also be uploaded. A system can also be interrogated to determine its tonal base or constrained to a particular tonal base. The pressing of a panel button may be indicated using a particular LED. The cart then expects a new preset in reply. Alternately, the base may be asked for the current preset and LED state, which can be initiated by the cart if it has detected a temporary (and now resolved) failure in the network.

This communication connection between a unit's master cart and one or more slave carts can only operate in the presence of some network topology to allow IP addressing between the carts (which at present means the presence of a base unit). Cart to cart communication allows a large architectural system to be musically coherent across all its output channels. It might also be necessary for the master cart of the system to relay some requests from the base to the slaves, rather than have the base address the slaves directly, if state change or synchronization constraints require it.

More generally, the modules shown and described may be implemented in computer-readable software code that is executed by one or more processors. The modules described may be implemented as a single module or in independent modules. The processor or processors include any device, system, or the like capable of executing computer-executable software code. The code may be stored on a processor, a memory device or on any other computer-readable storage medium. Alternatively, the software code may be encoded in a computer-readable electromagnetic signal, including electronic, electrical and optical signals. The code may be source code, object code or any other code performing or controlling the functionality described in this document. The computer-readable storage medium may be a magnetic storage disk such as a floppy disk, an optical disk such as a CD-ROM, semiconductor memory or any other physical object capable of storing program code or associated data.

Thus, as shown in the figures, a system for communication of multiple devices, either in physical proximity or remotely located is provided. The system establishes Master/Slave relationships between active systems and can force all slave systems to respond according to the master settings. The system also allows for the effective operation of the intercom through the LAN for sharing intercom parameters between different systems.

The sound screening system can respond to external acoustic energy that is either continuous or sporadic using multiple methods. The external sounds can be masked or their disturbing effect can be reduced using, for example, chords, arpeggios or preset sounds or music, as desired. Both, either, or neither the peaks nor RMS values in various critical bands associated with the sounds impinging on the sound screening system may be used to determine the acoustic energy emanating from the sound screening system. The sound screening system can be used to emit acoustic energy when the incident acoustic energy reaches a level to trigger an output from the sound screening system or may emit a continuous output that is dependent on the incident acoustic energy. This is to say that the output is closely related to and thus is adjusted in real-time or near real-time. The sound screening system can be used to emit acoustic energy at various times during a prescribed period whether or not incident acoustic energy reaches a level to trigger an output from the sound screening system. The sound screening system can be partially implemented by components which receive instructions from a computer readable medium or computer readable electromagnetic signal that contains computer-executable instructions for masking the environmental sounds.

It is therefore intended that the foregoing detailed description be regarded as illustrative rather than limiting, and that it be understood that it is the following claims, including all equivalents, that are intended to define the spirit and scope of this invention. For example, the geometries and material properties discussed herein and shown in the embodiments of the figures are intended to be illustrative only. Other variations may be readily substituted and combined to achieve particular design goals or accommodate particular materials or manufacturing processes.

1. An electronic sound screening system comprising:
a receiver on which acoustic energy impinges;
a converter that receives the acoustic energy from the receiver and converts the acoustic energy into an electrical signal;
an analyser that receives the electrical signal from the receiver, analyzes the electrical signal, and generates data analysis signals in response to the analyzed electrical signal;
a processor that produces sound signals based on the data analysis signals from the analyser in a plurality of individual critical bands; and
a sound generator that provides sound based on the sound signals.

2. The sound screening system of claim 1, wherein the sound signals are produced in all of the critical bands.

3. The sound screening system of claim 1, wherein the sound signals are produced in fewer than all of the critical bands.

4. The sound screening system of claim 1, wherein the receiver comprises sound sensing components, the sound generator comprises sound emitting components, and the sound sensing and sound emitting components are each positioned on a physical sound attenuating boundary to operate on a side of the boundary.

5. The sound screening system of claim 4, further comprising a control system through which a user can select the side of the boundary on which input sound is to be sensed and the side of the boundary on which sound is to be emitted.

6. The sound screening system of claim 4, wherein the sound sensing components include a pair of microphones mounted a short distance over a top edge of the boundary and pointing in opposite directions or mounted in pairs in the middle but opposite sides of the boundary, and the sound emitting components include a pair of speakers mounted on opposite sides of the boundary so as to emit sound primarily on the side of the boundary on which the speakers are placed.

7. The sound screening system of claim 4, wherein the system contains a DSP audio interface, an internal mixer/router of the DSP audio interface that is controlled using a Max/MSP external, a synthesiser driven via MIDI, and a control panel with a serial interface to perform external control, the system receives input from the sound environment using an array of the sound sensing components routed to the DSP audio interface via a mixer and an acoustic echo cancellation unit, and a response of the system is emitted into the sound environment by an array of the sound emitting units interfacing with the DSP audio interface via an array of amplifiers.

8. The sound screening system of claim 1, further comprising a flat panel speaker assembly containing multiple exciters separated by an acoustic medium, a panel excited in audible vibration, and acoustic foam in a gap between the acoustic medium and the panel.

9. The sound screening system of claim 8, wherein the acoustic medium is substantially planar and the exciters disposed on opposite sides of the acoustic medium do not overlap in a lateral direction of the flat panel speaker assembly.

10. The sound screening system of claim 8, wherein the acoustic medium contains a perpendicular bend and the exciters disposed on opposite sides of the acoustic medium overlap in a lateral direction of the flat panel speaker assembly.

11. An electronic sound screening system comprising:
a receiver on which acoustic energy impinges;
a converter that receives the acoustic energy from the receiver and converts the acoustic energy into an electrical signal;
an analyser that receives the electrical signal from the receiver, analyzes the electrical signal, and generates data analysis signals in response to the analyzed electrical signal;
a processor that produces sound signals, the sound signals selectable from at least one of: processing signals that are generated by processing of the data analysis signals, generative signals that are generated algorithmically and are adjusted by data analysis signals, and scripted signals that are predetermined by a user and are adjusted by the data analysis signals; and
a sound generator that provides sound based on the sound signals.

12. The sound screening system of claim 11, wherein the sound signals are mixed by a mixer prior to being supplied to the sound generator.

13. The sound screening system of claim 12, wherein the sound signals comprise at least one of filtered functional masker signals or harmonic masker signals.

14. The sound screening system of claim 12, wherein the generative signals comprise a chord voice, an arpeggio voice, a motive signal, a cloud signal of note events of varying densities, a control signal of control data for notes of random duration.

15. The sound screening system of claim 12, wherein the scripted signals comprise prerecorded sounds.

16. The sound screening system of claim 13, wherein the functional masker signals are based on 25 critical bands of the human ear.

17. The sound screening system of claim 11, wherein the processor produces the sound signals using at least one of a harmonic base, a system beat, harmonic settings generated therein and preset parameters supplied thereto.

18. The sound screening system of claim 11, further comprising a memory that stores results from the analyser and permits subsequent use by at least one of:
the analyzer, in generating the data analysis signals, and the processor, in producing the sound signals.

19. The sound screening system of claim 18, wherein the memory stores at least one of average root mean square (RMS) values of the data analysis signals for a predetermined period of time and the number of peak values of the data analysis signals for the predetermined period of time.

20. The sound screening system of claim 19, wherein the values stored are of a single critical band.

21. The sound screening system of claim 19, wherein the values stored are of multiple individual critical bands.

22. The sound screening system of claim 18, wherein the memory stores the results obtained from the analyser over multiple periods of time.

23. The sound screening system of claim 11, wherein the sound signals are activatable by the received acoustic energy.

24. The sound screening system of claim 11, further comprising a timer that induces the sound to be produced at one or more times during a prescribed period.

25. The sound screening system of claim 24, wherein the timer induces the sound to be produced during the prescribed period independent of whether the acoustic energy reaches a predetermined amplitude.

26. The sound screening system of claim 24, wherein the timer induces the sound to be produced in at least one predetermined critical band during the prescribed period.

27. The sound screening system of claim 26, wherein the timer induces the sound to be produced in fewer than all of the critical bands during the prescribed period.

28. The sound screening system of claim 11, further comprising a manually settable controller that provides user signals based on user selected inputs.

29. The sound screening system of claim 11, further comprising an intercom through which at least one user settable parameter is dynamically affected by at least one of the data analysis signals.

30. The sound screening system of claim 29, wherein multiple user settable parameters dynamically affect each other thereby forming a cascade of interactions.

31. The sound screening system of claim 11, wherein the sound signals are produced using outputs from soundsprites.

32. The sound screening system of claim 31, wherein parameters used by the soundsprites to produce the outputs are available to the soundsprites on one or more channels of an intercom.

33. The sound screening system of claim 32, wherein the same parameters that are available to different soundsprites on one of the channels of the intercom are useable differently by the different soundsprites.

34. The sound screening system of claim 32, wherein the parameters of different channels of the intercom are useable by one of the soundsprites and combinable to provide a particular output.

35. The sound screening system of claim 32, wherein a first output of a first of the soundsprites is able to affect a second output of a second of the soundsprites through one or more channels of the intercom.

36. The sound screening system of claim 35, further comprising a delay that permits the first output to affect the second output in real time or after a predetermined time delay as desired by a user.

37. The sound screening system of claim 32, wherein the output produced by one of the soundsprites is able to be affected in multiple ways when attributes of the same parameter on a channel of the intercom are different.

38. The sound screening system of claim 32, wherein different channels of the intercom are available to different numbers of components of the sound screening system.

39. The sound screening system of claim 11, wherein the sound signals comprise dependent signals that are dependent upon the received acoustic energy or independent signals that are independent of the received acoustic energy.

40. The sound screening system of claim 11, wherein the receiver comprises sound sensing components, the sound generator comprises sound emitting components, and the sound sensing and sound emitting components are each positioned on a physical sound attenuating boundary to operate on a side of the boundary.

41. The sound screening system of claim 40, further comprising a control system through which a user can select the side of the boundary on which input sound is to be sensed and the side of the boundary on which sound is to be emitted.

42. The sound screening system of claim 40, wherein the sound sensing components include a pair of microphones mounted a short distance over a top edge of the boundary and pointing in opposite directions or mounted in pairs in the middle but opposite sides of the boundary, and the sound emitting components include a pair of speakers mounted on opposite sides of the boundary so as to emit sound primarily on the side of the boundary on which the speakers are placed.

43. The sound screening system of claim 40, wherein the system contains a DSP audio interface, an internal mixer/router of the DSP audio interface that is controlled using a Max/MSP external, a synthesiser driven via MIDI, and a control panel with a serial interface to perform external control, the system receives input from the sound environment using an array of the sound sensing components routed to the DSP audio interface via a mixer and an acoustic echo cancellation unit, and a response of the system is emitted into the sound environment by an array of the sound emitting units interfacing with the DSP audio interface via an array of amplifiers.

44. The sound screening system of claim 11, further comprising a flat panel speaker assembly containing multiple exciters separated by an acoustic medium, a panel excited in audible vibration, and acoustic foam in a gap between the acoustic medium and the panel.

45. The sound screening system of claim 44, wherein the acoustic medium is substantially planar and the exciters disposed on opposite sides of the acoustic medium do not overlap in a lateral direction of the flat panel speaker assembly.

46. The sound screening system of claim 44, wherein the acoustic medium contains a perpendicular bend and the exciters disposed on opposite sides of the acoustic medium overlap in a lateral direction of the flat panel speaker assembly.

47. An electronic sound screening system comprising:
a local user interface through which a local user enters local user inputs to change a state of the sound screening system;
a remote user interface through which a remote user enters remote user inputs to change the state of the sound screening system;
a receiver on which acoustic energy impinges;
a converter that receives the acoustic energy from the receiver and converts the acoustic energy into an electrical signal;
an analyser that receives the electrical signal from the receiver, analyzes the electrical signal, and generates data analysis signals in response to the analyzed electrical signal;
a processor that produces sound signals based on the data analysis signals from the analyser and a weighed combination of the local and remote user inputs; and
a sound generator that provides sound based on the sound signals.

48-52. (canceled)

53. The sound screening system of claim 47, further comprising a voting module through which multiple users transmit parameters to change the state of the sound screening system.

54. The sound screening system of claim 53, wherein the voting module alters the state of the sound screening system depending on different weights given to the different users.

55. The sound screening system of claim 54, wherein the different weights depend on proximity of the different users to the sound screening system.

56-120. (canceled)
